(19) United States
(12) Patent Application Publication
(10) Pub. No.: US 2018/0280410 A1
(43) Pub. Date: Oct. 4, 2018

(54) NATURAL COMBINATION HORMONE REPLACEMENT FORMULATIONS AND THERAPIES

(71) Applicant: TherapeuticsMD, Inc., Boca Raton, FL (US)

(72) Inventors: Julia M. Amadio, Boca Raton, FL (US); Brian A. Bernick, Boca Raton, FL (US); Sebastian Mirkin, Boca Raton, FL (US)

(21) Appl. No.: 15/832,750

(22) Filed: Dec. 5, 2017

Related U.S. Application Data
(60) Provisional application No. 62/481,060, filed on Apr. 3, 2017.

(51) Int. Cl. A61K 31/566; A61K 31/57; A61P 5/30

(52) U.S. Cl. CPC A61K 31/566; A61K 2300/00; A61P 5/30; A61K 31/57

(57) ABSTRACT

Pharmaceutical compositions for co-administering estradiol and progesterone to a human subject having vasomotor symptoms associated with estrogen deficiency are provided. In some embodiments, the pharmaceutical composition produces certain pharmacokinetic parameters when the composition is administered to the subject.

### CROSS-REFERENCE TO RELATED APPLICATIONS

This application claims priority to U.S. Provisional Patent Application No. 62/481,060, filed Apr. 3, 2017, the entire contents of which are incorporated by reference herein.

### FIELD OF THE INVENTION

This application relates to pharmaceutical compositions and methods for hormone replacement therapy.

### BACKGROUND OF THE INVENTION

Hormone Replacement Therapy (HRT) is a medical treatment that involves the use of one or more of a group of medications designed to increase hormone levels in women who lack adequate hormone production. HRT can mitigate and prevent symptoms caused by diminished circulating estrogen and progesterone hormones in a pre-menopausal, peri-menopausal, menopausal, or post-menopausal subject.

### BRIEF SUMMARY OF THE INVENTION

In one aspect, pharmaceutical compositions for co-administering estradiol and progesterone to a subject in need of natural hormone replacement therapies are provided. In some embodiments, the pharmaceutical composition is for co-administering estradiol and progesterone to a human subject having vasomotor symptoms associated with estrogen deficiency.

In some embodiments, the pharmaceutical composition comprises 1 mg of estradiol and 100 mg of progesterone, and when administered to the subject, produces in a serum sample from the subject, one or more pharmacokinetic parameters selected from:
(i) a steady state estradiol concentration that is from 42.29 pg/mL to 45.58 pg/mL;
(ii) a steady state estrone concentration that is from 213.79 pg/mL to 241.57 pg/mL; and
(iii) a steady state progesterone concentration that is from 386.53 pg/mL to 547.83 pg/mL.

In some embodiments, administration of the composition to the subject produces a steady state estradiol concentration that is from 42.29 pg/mL to 45.58 pg/mL. In some embodiments, administration of the composition to the subject produces both of a steady state estradiol concentration that is from 42.29 pg/mL to 45.58 pg/mL and a steady state progesterone concentration that is from 386.53 pg/mL to 547.83 pg/mL. In some embodiments, administration of the composition to the subject produces both of a steady state estradiol concentration that is from 42.29 pg/mL to 45.58 pg/mL and a steady state estrone concentration that is from 213.79 pg/mL to 241.57 pg/mL. In some embodiments, administration of the composition to the subject produces each of: (i) a steady state estradiol concentration that is from 42.29 pg/mL to 45.58 pg/mL; (ii) a steady state estrone concentration that is from 213.79 pg/mL to 241.57 pg/mL; and (iii) a steady state progesterone concentration that is from 386.53 pg/mL to 547.83 pg/mL.

In some embodiments, the pharmaceutical composition comprises 0.5 mg of estradiol and 100 mg of progesterone, and when administered to the subject, produces in a serum sample from the subject, one or more pharmacokinetic parameters selected from:
(i) a steady state estradiol concentration that is from 23.03 pg/mL to 27.37 pg/mL;
(ii) a steady state estrone concentration that is from 113.59 pg/mL to 132.08 pg/mL; and
(iii) a steady state progesterone concentration that is from 386.53 pg/mL to 547.83 pg/mL.

In some embodiments, administration of the composition to the subject produces a steady state estradiol concentration that is from 23.03 pg/mL to 27.37 pg/mL. In some embodiments, administration of the composition to the subject produces both of a steady state estradiol concentration that is from 23.03 pg/mL to 27.37 pg/mL and a steady state progesterone concentration that is from 386.53 pg/mL to 547.83 pg/mL. In some embodiments, administration of the composition to the subject produces both of a steady state estradiol concentration that is from 23.03 pg/mL to 27.37 pg/mL and a steady state estrone concentration that is from 113.59 pg/mL to 132.08 pg/mL. In some embodiments, administration of the composition to the subject produces each of: (i) a steady state estradiol concentration that is from 23.03 pg/mL to 27.37 pg/mL; (ii) a steady state estrone concentration that is from 113.59 pg/mL to 132.08 pg/mL; and (iii) a steady state progesterone concentration that is from 386.53 pg/mL to 547.83 pg/mL.

In some embodiments, the pharmaceutical composition comprises 0.5 mg of estradiol and 50 mg of progesterone, and when administered to the subject, produces in a serum sample from the subject, one or more pharmacokinetic parameters selected from:
(i) a steady state estradiol concentration that is from 23.03 pg/mL to 27.37 pg/mL;
(ii) a steady state estrone concentration that is from 113.59 pg/mL to 132.08 pg/mL; and
(iii) a steady state progesterone concentration that is from 181.41 pg/mL to 247.17 pg/mL.

In some embodiments, administration of the composition to the subject produces a steady state estradiol concentration that is from 23.03 pg/mL to 27.37 pg/mL. In some embodiments, administration of the composition to the subject produces both of a steady state estradiol concentration that is from 23.03 pg/mL to 27.37 pg/mL and a steady state progesterone concentration that is from 181.41 pg/mL to 247.17 pg/mL. In some embodiments, administration of the composition to the subject produces both of a steady state estradiol concentration that is from 23.03 pg/mL to 27.37 pg/mL and a steady state estrone concentration that is from 113.59 pg/mL to 132.08 pg/mL. In some embodiments, administration of the composition to the subject produces each of: (i) a steady state estradiol concentration that is from 23.03 pg/mL to 27.37 pg/mL; (ii) a steady state estrone concentration that is from 113.59 pg/mL to 132.08 pg/mL; and (iii) a steady state progesterone concentration that is from 181.41 pg/mL to 247.17 pg/mL.

In some embodiments, the pharmaceutical composition comprises 0.25 mg of estradiol and 50 mg of progesterone, and when administered to the subject, produces in a serum sample from the subject, one or more pharmacokinetic parameters selected from:
(i) a steady state estradiol concentration that is from 15.06 pg/mL to 18.50 pg/mL;
(ii) a steady state estrone concentration that is from 69.02 pg/mL to 73.43 pg/mL; and
(iii) a steady state progesterone concentration that is from 181.41 pg/mL to 247.17 pg/mL.

In some embodiments, administration of the composition to the subject produces a steady state estradiol concentration that is from 15.06 pg/mL to 18.50 pg/mL. In some embodiments, administration of the composition to the subject produces both of a steady state estradiol concentration that is from 15.06 pg/mL to 18.50 pg/mL and a steady state progesterone concentration that is from 181.41 pg/mL to 247.17 pg/mL. In some embodiments, administration of the composition to the subject produces both of a steady state estradiol concentration that is from 15.06 pg/mL to 18.50 pg/mL and a steady state estrone concentration that is from 69.02 pg/mL to 73.43 pg/mL. In some embodiments, administration of the composition to the subject produces each of: (i) a steady state estradiol concentration that is from 15.06 pg/mL to 18.50 pg/mL; (ii) a steady state estrone concentration that is from 69.02 pg/mL to 73.43 pg/mL; and (iii) a steady state progesterone concentration that is from 181.41 pg/mL to 247.17 pg/mL.

In some embodiments, the pharmacokinetic parameters are measured after at least 4 weeks of daily administration of the pharmaceutical composition to the subject, e.g., after at least 6 weeks, 8 weeks, 10 weeks, 12 weeks, 16 weeks, 20 weeks, or 24 weeks of daily administration of the pharmaceutical composition to the subject.

In some embodiments, the pharmaceutical composition comprises a solubilizing agent. In some embodiments, the pharmaceutical composition comprises solubilized estradiol. In some embodiments, the pharmaceutical composition comprises suspended progesterone. In some embodiments, each of the solubilized estradiol and the suspended progesterone are present in the solubilizing agent. In some embodiments, at least about 90% of the estradiol is solubilized in the solubilizing agent.

In some embodiments, the pharmaceutical composition comprises: solubilized estradiol, suspended progesterone, and a solubilizing agent, wherein the solubilizing agent is a medium chain (C6-C12) oil and wherein the pharmaceutical composition, when administered to a subject, produces in a plasma sample from the subject one or more pharmacokinetic parameters as described herein (e.g., a steady state concentration, an area under the curve (AUC)₍₀₋ₜ₎ or a Cₘₐₓ for estradiol, progesterone, estrone, or total estrone as described herein, e.g., in Tables 18-21 and Tables 25-27).

In some embodiments, the pharmaceutical composition comprises a solubilizing agent that comprises a glyceride of at least one C6-C12 fatty acid. In some embodiments, the solubilizing agent comprises monoglycerides, diglycerides, triglycerides, or a combination thereof, wherein the monoglycerides, diglycerides, and triglycerides are predominantly of C6-C12 fatty acid chain lengths. In some embodiments, the glyceride ester is a mixture of mono- and diglycerides (e.g., glyceryl caprylate/caprate). In some embodiments, the fatty acid is predominantly a C8 to C10 fatty acid. In some embodiments, the pharmaceutical composition further comprises a surfactant (e.g., lauroyl polyoxyglyceride).

In another aspect, methods of treating a subject are provided. In some embodiments, the subject has a condition that is caused at least in part by an estrogen deficiency (e.g., one or more symptoms of menopause, such as vasomotor symptoms). In some embodiments, the method comprises administering to the subject a pharmaceutical composition comprising estradiol and progesterone as described herein (e.g., 1 mg of estradiol and 100 mg of progesterone; 0.5 mg of estradiol and 100 mg of progesterone; 0.5 mg of estradiol and 50 mg of progesterone; or 0.25 mg of estradiol and 50 mg of progesterone), wherein administration of the pharmaceutical composition produces, in a sample (e.g., serum sample) from the subject, one or more pharmacokinetic parameters as described herein, e.g., a steady state concentration for one or more of estradiol, estrone, or progesterone.

In some embodiments, administration of the composition to the subject further produces in the subject a reduction in the frequency and/or severity of one or more symptoms of menopause. In some embodiments, administration of the composition to the subject further produces in the subject a reduction in the frequency and/or severity of one or more moderate to severe vasomotor symptoms associated with menopause. In some embodiments, the vasomotor symptoms are selected from the group consisting of hot flashes or flushes, night sweats, sweating, sleep disturbances, and combinations thereof.

In some embodiments, the subject is female. In some embodiments, the subject is a woman having a uterus. In some embodiments, prior to treatment, the subject has a serum estradiol of ≤10 pg/mL.

In still another aspect, pharmaceutical compositions for use in a method of treating a disease or condition that is caused at least in part by an estrogen deficiency are provided. In some embodiments, the pharmaceutical composition comprises estradiol and progesterone as described herein (e.g., 1 mg of estradiol and 100 mg of progesterone; 0.5 mg of estradiol and 100 mg of progesterone; 0.5 mg of estradiol and 50 mg of progesterone; or 0.25 mg of estradiol and 50 mg of progesterone), wherein administration of the pharmaceutical composition produces, in a sample (e.g., serum sample) from the subject, one or more pharmacokinetic parameters as described herein, e.g., a steady state concentration for one or more of estradiol, estrone, or progesterone.

In some embodiments, a pharmaceutical composition for use in a method of treating a disease or condition that is caused at least in part by an estrogen deficiency (e.g., one or more symptoms of menopause) comprises estradiol at a dosage of about 1 mg and progesterone at a dosage of about 100 mg, and produces one or more of the following pharmacokinetic values when administered to a subject (e.g., after about 4 weeks of daily administration): (i) a steady state estradiol concentration that is from 42.29 pg/mL to 45.58 pg/mL; (ii) a steady state estrone concentration that is from 213.79 pg/mL to 241.57 pg/mL; and (iii) a steady state progesterone concentration that is from 386.53 pg/mL to 547.83 pg/mL.

In some embodiments, a pharmaceutical composition for use in a method of treating a disease or condition that is caused at least in part by an estrogen deficiency (e.g., one or more symptoms of menopause) comprises estradiol at a dosage of about 0.50 mg and progesterone at a dosage of about 100 mg, and produces one or more of the following pharmacokinetic values when administered to a subject (e.g., after about 4 weeks of daily administration): (i) a steady state estradiol concentration that is from 23.03 pg/mL to 27.37 pg/mL; (ii) a steady state estrone concentration that is from 113.59 pg/mL to 132.08 pg/mL; and (iii) a steady state progesterone concentration that is from 386.53 pg/mL to 547.83 pg/mL.

In some embodiments, a pharmaceutical composition for use in a method of treating a disease or condition that is caused at least in part by an estrogen deficiency (e.g., one or more symptoms of menopause) comprises estradiol at a dosage of about 0.50 mg and progesterone at a dosage of about 50 mg, and produces one or more of the following pharmacokinetic values when administered to a subject (e.g., after about 4 weeks of daily administration): (i) a steady state estradiol concentration that is from 23.03 pg/mL to 27.37 pg/mL; (ii) a steady state estrone concentration that is from 113.59 pg/mL to 132.08 pg/mL; and (iii) a steady state progesterone concentration that is from 181.41 pg/mL to 247.17 pg/mL.

In some embodiments, a pharmaceutical composition for use in a method of treating a disease or condition that is caused at least in part by an estrogen deficiency (e.g., one or more symptoms of menopause) comprises estradiol at a dosage of about 0.25 mg and progesterone at a dosage of about 50 mg, and produces one or more of the following pharmacokinetic values when administered to a subject (e.g., after about 4 weeks of daily administration): (i) a steady state estradiol concentration that is from 15.06 pg/mL to 18.50 pg/mL; (ii) a steady state estrone concentration that is from 69.02 pg/mL to 73.43 pg/mL; and (iii) a steady state progesterone concentration that is from 181.41 pg/mL to 247.17 pg/mL.

### BRIEF DESCRIPTION OF THE DRAWINGS

FIG. 1 illustrates a semilogarithmic plot of mean plasma concentration (pg/ml) over time (hrs) for estradiol.

FIG. 2 illustrates a semilogarithmic plot of mean plasma concentration (ng/ml) over time (hrs) for progesterone.

FIG. 3 illustrates a semilogarithmic plot of mean plasma concentration (pg/ml) over time (hrs) for estrone.

FIG. 4 illustrates a semilogarithmic plot of mean plasma concentration (ng/ml) over time (hrs) for total estrone.

FIG. 5 illustrates mean change from baseline in weekly frequency of moderate to severe hot flashes for weeks 1 to 12.

FIG. 6 illustrates mean change from baseline in weekly severity of moderate to severe hot flashes for weeks 1 to 12.

### DETAILED DESCRIPTION OF THE INVENTION

In the following detailed description of embodiments of this disclosure, reference is made to the accompanying drawings in which like references indicate similar elements, and in which is shown, by way of illustration, specific embodiments in which this disclosure may be practiced. These embodiments are described in sufficient detail to enable those skilled in the art to practice this disclosure, and it is to be understood that other embodiments may be utilized and that other changes may be made without departing from the scope of this disclosure. The following detailed description is, therefore, not to be taken in a limiting sense, and the scope of this disclosure is defined only by the appended claims. As used in this disclosure, the term “or” shall be understood to be defined as a logical disjunction (i.e., and/or) and shall not indicate an exclusive disjunction unless expressly indicated as such with the term “either,” “unless,” “alternatively,” and words of similar effect. The singular forms “a,” “an,” and “the” include plural referents unless the context clearly dictates otherwise.

### I. DEFINITIONS

The term “about” refers to ±10% of the noted value, unless otherwise specified, and unless the upper bound of the range would exceed 100% of the pharmaceutical composition, in which case the upper limit of the range is limited to 99.9%. Thus, and by way of example only, a pharmaceutical composition including about 10 weight percent of a given compound could have from 9 to 11 weight percent of the compound. Similarly, a pharmaceutical composition including about 95 weight percent of a given compound could have from 85.5 to 99.9 weight percent of the compound in the pharmaceutical composition.

The term “area under the curve” (“AUC”) refers to the area under the curve defined by changes in the blood, plasma, or serum concentration of an active pharmaceutical ingredient (e.g., estradiol or progesterone), or one or more metabolites of the active pharmaceutical ingredient, over time following the administration of a dose of the active pharmaceutical ingredient. “AUC_{0-∞}” is the area under the concentration-time curve extrapolated to infinity following the administration of a dose. “AUC₀₋ₜ” is the area under the concentration-time curve from time zero to time t following the administration of a dose, wherein t is the last time point with measurable concentration.

The term “Cₘₐₓ” refers to the maximum value of blood, plasma, or serum concentration shown on the curve that represents changes in blood, plasma, or serum concentrations of an active pharmaceutical ingredient (e.g., progesterone or estradiol), or one or more metabolites of the active pharmaceutical ingredient, over time.

The term “steady state concentration” or “Cₛₛ” refers to the blood, plasma, or serum concentration of an active pharmaceutical ingredient (e.g., progesterone or estradiol) at the time when the rate of intake of the active pharmaceutical ingredient is in equilibrium with the rate of elimination of the active pharmaceutical ingredient.

The term “Tₘₐₓ” refers to the time that it takes for the blood, plasma, or serum concentration of an active pharmaceutical ingredient (e.g., estradiol or progesterone), or of one or more metabolites of the active pharmaceutical ingredient, to reach the maximum value.

Collectively, AUC, Cₘₐₓ, Cₛₛ, and, optionally, Tₘₐₓ are the principal pharmacokinetic parameters that can characterize the pharmacokinetic response of a particular drug product, such as progesterone or estradiol, in an animal, especially a mammal, including human, subject.

An “active pharmaceutical ingredient” (API), as used herein, means the active compound or compounds used in formulating a drug product. APIs are generally safe for administering to animals, especially mammals, including humans, according to established governmental standards, including those promulgated by the United States Food and Drug Administration.

The term “bioavailability” has the meaning as defined in 21 C.F.R. § 320.1(a) and refers to the rate and extent to which an API or active ingredient or active moiety is absorbed from a drug product and becomes available at the site of action. For drug products that are not intended to be absorbed into the bloodstream, bioavailability may be assessed by measurements intended to reflect the rate and extent to which the API or active ingredient or active moiety becomes available at the site of action. For example, bioavailability can be measured as the amount of API in the blood (whole blood, serum, or plasma) as a function of time. In embodiments, the amount of API is measured in blood plasma. Pharmacokinetic (PK) parameters such as AUC, Cₘₐₓ, or Tₘₐₓ may be used to measure and assess bioavailability.

The term “bioequivalent” has the meaning as defined in 21 C.F.R. § 320.1(e) and refers to the absence of a significant difference in the rate and extent to which the API or active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms or modified release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the AUC, Cₘₐₓ, or optionally Tₘₐₓ is within 80.00% to 125.00%.

The term “bio-identical hormone” or “body-identical hormone” refers to an active pharmaceutical ingredient that is structurally identical to a hormone naturally or endogenously found in the human body (e.g., estradiol and progesterone).

The term “estrogen” refers to a group of several female sex hormones produced primarily by the ovaries, including estradiol, estrone, and estriol. As used herein, unless otherwise specified, estrogen refers to estradiol.

The term “estradiol” refers to (17β)-estra-1,3,5(10)-triene-3,17-diol. Estradiol is also interchangeably called 17β-estradiol, estradiol, or E2, and is found endogenously in the human body. As used herein, estradiol refers to the bio-identical or body-identical form of estradiol found in the human body having the structure:

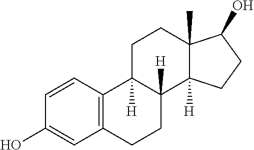

As used herein, unless specified, estradiol includes estradiol in anhydrous or hemihydrate forms. For the purposes of this disclosure, the anhydrous form or the hemihydrate form can be substituted for the other by accounting for the water or lack of water according to well-known and understood techniques.

The term “solubilized estradiol” means that the estradiol or a portion thereof is solubilized or dissolved in the solubilizing agents or the formulations disclosed herein. Solubilized estradiol may include estradiol that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the estradiol is “fully solubilized” with all or substantially all of the estradiol being solubilized or dissolved in the solubilizing agent. Fully solubilized estradiol may include estradiol that is about 97% solubilized, about 98% solubilized, about 99% solubilized or about 100% solubilized. Solubility can be expressed as a mass fraction (% w/w, which is also referred to as wt %).

The term “progesterone” refers to pregn-4-ene-3,20-dione. Progesterone is also interchangeably called P4 and is found endogenously in the human body. As used herein, progesterone refers to the bio-identical or body-identical form of progesterone found in the human body having the structure:

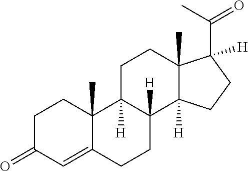

The term “solubilized progesterone” means that the progesterone or a portion thereof is solubilized or dissolved in the solubilizing agents or the formulations disclosed herein disclosed herein. In some embodiments, the progesterone is “partially solubilized” with a portion of the progesterone being solubilized or dissolved in the solubilizing agent and a portion of the progesterone being suspended in the solubilizing agent. Partially solubilized progesterone may include progesterone that is about 1% solubilized, about 5% solubilized, about 10% solubilized, about 15% solubilized, about 20% solubilized, about 30% solubilized, about 40% solubilized, about 50% solubilized, about 60% solubilized, about 70% solubilized, about 80% solubilized, about 85% solubilized, about 90% solubilized or about 95% solubilized. In other embodiments, the progesterone is “fully solubilized” with all or substantially all of the progesterone being solubilized or dissolved in the solubilizing agent. Fully solubilized progesterone may include progesterone that is about 97% solubilized, about 98% solubilized, about 99% solubilized or about 100% solubilized. Solubility can be expressed as a mass fraction (% w/w, which is also referred to as wt %).

The terms “micronized progesterone” and “micronized estradiol,” as used herein, include micronized progesterone and micronized estradiol, respectively, having an X50 particle size value below about 15 microns or having an X90 particle size value below about 25 microns. The term “X50” means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

The term “solubilizing agent” refers to an agent or combination of agents that solubilize an active pharmaceutical ingredient (e.g., estradiol or progesterone). For example and without limitation, suitable solubilizing agents include medium chain oils and other solvents and co-solvents that solubilize or dissolve an active pharmaceutical ingredient to a desirable extent. Solubilizing agents suitable for use in the pharmaceutical compositions disclosed herein are pharmaceutical grade solubilizing agents (e.g., pharmaceutical grade medium chain oils). It will be understood by those of skill in the art that other excipients or components can be added to or mixed with the solubilizing agent to enhance the properties or performance of the solubilizing agent or resulting pharmaceutical composition. Examples of such excipients include, but are not limited to, surfactants, emulsifiers, thickeners, colorants, flavoring agents, etc. In some embodiments, the solubilizing agent is a medium chain oil and, in some other embodiments, the medium chain oil is combined with a co-solvent(s) or other excipient(s).

The term “medium chain” is used to describe the aliphatic chain length of fatty acid containing molecules. “Medium chain” specifically refers to fatty acids, fatty acid esters, or fatty acid derivatives that contain fatty acid aliphatic tails or carbon chains that contain, for example, 6 to 14 carbon atoms, 8 to 12 carbon atoms, or 8 to 10 carbon atoms.

The terms “medium chain fatty acid” and “medium chain fatty acid derivative” are used to describe fatty acids or fatty acid derivatives with aliphatic tails (i.e., carbon chains) having 6 to 14 carbons. Fatty acids consist of an unbranched aliphatic tail attached to a carboxylic acid functional group. Fatty acid derivatives include, for example, fatty acid esters and fatty acid containing molecules, including, without limitation, mono-, di- and triglycerides that include components derived from fatty acids as well as fatty acid esters of ethylene or propylene glycol. Those of skill will appreciate that the aliphatic tails can be saturated or unsaturated (one or more double bonds between carbon atoms). In some embodiments, the aliphatic tails are saturated (i.e., no double bonds between carbon atoms). Medium chain fatty acids or medium chain fatty acid derivatives include those with aliphatic tails having 6-14 carbons, including those that are C6-C14, C6-C12, C8-C14, C8-C12, C6-C10, C8-C10, or others. In embodiments, medium chain fatty acids or medium chain fatty acid derivatives are those that are saturated. Examples include, without limitation, caproic acid, caprylic acid, capric acid, lauric acid, myristic acid, and derivatives thereof. In certain embodiments, the medium chain fatty acids used to prepare the various medium chain oils described herein are C8, C10, or a combination thereof.

The term “oil,” as used herein, refers to any pharmaceutically acceptable oil, especially medium chain oils, and specifically excluding peanut oil, that can suspend or solubilize bioidentical progesterone or estradiol, including starting materials or precursors thereof, including micronized progesterone and/or micronized estradiol as described herein.

The term “medium chain oil” refers to an oil wherein the composition of the fatty acid fraction of the oil is substantially medium chain (i.e., C6 to C14) fatty acids, i.e., the composition profile of fatty acids in the oil is substantially medium chain. As used herein, “substantially” means that between 20% and 100% (inclusive of the upper and lower limits) of the fatty acid fraction of the oil is made up of medium chain fatty acids, i.e., fatty acids with aliphatic tails (i.e., carbon chains) having 6 to 14 carbons. In some embodiments, about 25%, about 30%, about 35%, about 40%, about 45%, about 50%, about 55%, about 60%, about 65%, about 70%, about 75%, about 85%, about 90% or about 95% of the fatty acid fraction of the oil is made up of medium chain fatty acids. Those of skill in the art will readily appreciate that the terms “alkyl content” or “alkyl distribution” of an oil can be used in place of the term “fatty acid fraction” of an oil in characterizing a given oil or solubilizing agent, and these terms are used interchangeably herein. As such, medium chain oils suitable for use in the pharmaceutical compositions disclosed herein include medium chain oils wherein the fatty acid fraction of the oil is substantially medium chain fatty acids, or medium chain oils wherein the alkyl content or alkyl distribution of the oil is substantially medium chain alkyls, e.g., C6-C14 alkyls, but also including, for example, C6-C12 alkyls, C8-C12 alkyls, and C8-C10 alkyls. It will be understood by those of skill in the art that the medium chain oils suitable for use in the pharmaceutical compositions disclosed herein are pharmaceutical grade (e.g., pharmaceutical grade medium chain oils). Examples of medium chain oils include, for example and without limitation, medium chain fatty acids, medium chain fatty acid esters of glycerol (e.g., for example, mono-, di-, and triglycerides), medium chain fatty acid esters of propylene glycol, medium chain fatty acid derivatives of polyethylene glycol, and combinations thereof.

The term “ECN” or “equivalent carbon number” means the sum of the number of carbon atoms in the fatty acid chains of an oil, and can be used to characterize an oil as, for example, a medium chain oil or a long-chain oil. For example, tripalmitin (tripalmitic glycerol), which is a simple triglyceride containing three fatty acid chains of 16 carbon atoms, has an ECN of 3×16=48. Conversely, a triglyceride with an ECN=40 may have “mixed” fatty acid chain lengths of 8, 16, and 16; 10, 14, and 16; 8, 14, and 18; etc. Naturally occurring oils are frequently “mixed” with respect to specific fatty acids, but tend not to contain both long chain fatty acids and medium chain fatty acids in the same glycerol backbone. Thus, triglycerides with ECNs of 21-42 typically contain predominantly medium chain fatty acids; while triglycerides with ECNs of greater than 43 typically contain predominantly long chain fatty acids. For example, the ECN of corn oil triglyceride in the US Pharmacopeia (USP) would be in the range of 51-54. Medium chain diglycerides with ECNs of 12-28 will often contain predominantly medium chain fatty acids, while diglycerides with ECNs of 32 or greater will typically contain predominantly long chain fatty acids. Monoglycerides will have an ECN that matches the chain length of the sole fatty acid chain. Thus, monoglyceride ECNs in the range of 6-14 contain mainly medium chain fatty acids, and monoglycerides with ECNs 16 or greater will contain mainly long chain fatty acids.

The average ECN of a medium chain triglyceride oil is typically 21-42. For example, as listed in the USP, medium chain triglycerides have the following composition as the exemplary oil set forth in the table below:

| Fatty Acid Tail Length | % of Oil | Exemplary Oil |
|---|---|---|
| 6 | ≤2.0 | 2.0 |
| 8 | 50.0-80.0 | 70.0 |
| 10 | 20.0-50.0 | 25.0 |
| 12 | ≤3.0 | 2.0 |
| 14 | ≤1.0 | 1.0 |

and would have an average ECN of 3*[(6*0.02)+(8*0.070)+(10*0.25)+(12*0.02)+(14*0.01)]=25.8. The ECN of the exemplary medium chain triglycerides oil can also be expressed as a range (per the ranges set forth in the USP) of 24.9-27.0. For oils that have mixed mono-, di-, and triglycerides, or single and double fatty acid glycols, the ECN of the entire oil can be determined by calculating the ECN of each individual component (e.g., C8 monoglycerides, C8 diglycerides, C10 monoglycerides, and C10 diglycerides) and taking the sum of the relative percentage of the component multiplied by the ECN normalized to a monoglyceride for each component. For example, the oil having C8 and C10 mono- and diglycerides shown in the table below has an ECN of 8.3, and is thus a medium chain oil:

| | | ECN as % of Oil | ECN as % of Oil |
|---|---|---|---|
| Fatty Acid Chain | % of | [(chain length) × | Normalized to |
| Length | Oil | (% in oil)] | Monoglyceride |
| C8 monoglyceride | 47 | 8 × 0.47 = 3.76 | 3.76 |
| C10 monoglyceride | 8 | 10 × 0.08 = 0.8 | 0.8 |
| C8 diglyceride | 38 | 2 × (8 × 0.38) = 6.08 | 6.08/2 = 3.04 |
| C10 diglyceride | 7 | 2 × (10 × 0.07) = 1.4 | 1.4/2 = 0.7 |
| OIL ECN | | | 8.3 |
| (normalized to | | | |
| monoglycerides) | | | |

Expressed differently, ECN can be calculated as each chain length in the composition multiplied by its relative percentage in the oil: (8*0.85)+(10*0.15)=8.3.

The term “excipients,” as used herein, refers to non-active pharmaceutical ingredients such as solubilizing agents, anti-oxidants, oils, lubricants, and others used in formulating pharmaceutical products.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subject parameters, including the results of a physical examination, neuropsychiatric examinations, or psychiatric evaluation.

### II. PHARMACEUTICAL COMPOSITIONS

In one aspect, this disclosure relates to pharmaceutical compositions for co-administering estradiol and progesterone to a human subject in need thereof, e.g., a subject having vasomotor symptoms associated with estrogen deficiency. In some embodiments, the composition comprises estradiol, progesterone, and a solubilizing agent (e.g., a medium chain oil, e.g., a C6-C12 oil). In some embodiments, a pharmaceutical composition comprising estradiol, progesterone, and a solubilizing agent as described herein, when administered to a subject or a population of subjects, produces one or more AUC, Cₘₐₓ, or Tₘₐₓ parameters for estradiol, progesterone, estrone, or total estrone as described below.

### Formulations of Estradiol and Progesterone Compositions

In some embodiments, a pharmaceutical composition for use as described herein comprises solubilized estradiol with suspended progesterone; solubilized estradiol with both partially solubilized progesterone and partially suspended progesterone; or solubilized estradiol with fully solubilized progesterone. In some embodiments, the composition comprises solubilized estradiol and suspended progesterone. The underlying formulation concepts provided herein may be used with other natural or synthetic forms of estradiol and progesterone, although the natural or bio-identical forms of estradiol and progesterone are preferred.

In some embodiments, the composition comprises estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg. In some embodiments, the composition comprises progesterone at a dosage of about 25, 50, 75, 100, 125, 150, 175, 200, 250, 300, 350, or 400 mg.

In some embodiments, estradiol is solubilized. Solubilized estradiol may include estradiol that is approximately 80% to 100% soluble in a solubilizing agent, including specifically embodiments that are: 80%, 81%, 82%, 83%, 84%, 85%, 86%, 87%, 88%, 89%, 90%, 91%, 92%, 93%, 94%, 95%, 96%, 97%, 98%, 99%, or 100% soluble in a solubilizing agent. Solubility may be expressed as a mass fraction (% w/w, also referred to as wt %). In some embodiments, estradiol is micronized. In some embodiments, micronized estradiol has an X50 particle size value of less than about 15 microns, less than about 10 microns, less than about 5 microns or less than about 3 microns. In some embodiments, micronized estradiol has an X90 particle size value of less than about 25 microns, less than about 20 microns, or less than about 15 microns. In some embodiments, the composition comprises micronized and partially solubilized estradiol.

In some embodiments, the composition comprises micronized progesterone. The progesterone active pharmaceutical ingredient may be micronized via any one of the multiple methods typically utilized by the ordinarily skilled artisan. In various embodiments, micronized progesterone has an X50 particle size value of less than about 15 microns, less than about 10 microns, less than about 5 microns or less than about 3 microns. In various embodiments, micronized progesterone has an X90 particle size value of less than about 25 microns, less than about 20 microns, or less than about 15 microns. Particle size may be determined in any suitable manner. For example, a Beckman Coulter LS 13 320 Laser Diffraction Particle Size Analyzer (the “Beckman Device”) may be used to determine particle size.

Estradiol and progesterone compositions and methods of preparing such compositions are described in U.S. Pat. No. 8,633,178; U.S. Publication No. 2013/0129818; U.S. Publication No. 2013/0338123; International Publication No. WO 2013/078422; and International Publication No. WO 2013/192251; each of which is incorporated by reference in its entirety.

Solubilizing Agents

Estradiol and progesterone compositions of the present disclosure are prepared via blending with a solubilizing agent. In some embodiments, the solubilizing agent is a pharmaceutically acceptable oil that comprises a medium chain oil. In some embodiments, the solubilizing agent is a medium chain oil comprised substantially of C6-C12 medium chains, e.g., at least 20%, at least 30%, at least 40%, at least 50%, at least 60%, at least 70%, at least 80%, or at least 90% of the chains present in the oil are C6-C12. In some embodiments, the oil comprises at least one medium chain fatty acid such as medium chain fatty acids having at least one mono-, di-, or triglyceride, or derivatives thereof, or combinations thereof. In some embodiments, the medium chain oil comprises at least one medium chain fatty acid or propylene glycol, polyethylene glycol, or glyceride having esters of medium chain fatty acids. In some embodiments, the solubilizing agent is not peanut oil.

In some embodiments, oils used to solubilize estradiol and to suspend, partially suspend and partially solubilize, or fully solubilize progesterone include medium chain fatty acid esters, (e.g., esters of glycerol, polyethylene glycol, or propylene glycol) and mixtures thereof. In some embodiments, the medium chain fatty acids are C6, C8, C10, C12, C6-C12, C8-C12, C6-C10, C8-C10, or C10-C12 fatty acids. In some embodiments, the medium chain fatty acids are saturated, or predominantly saturated, e.g., greater than about 50% saturated, greater than about 60% saturated, or greater than about 75% saturated. In some embodiments, a solubilizing agent comprises predominantly medium chain length, saturated fatty acids or derivatives thereof, specifically predominantly C8 to C12 saturated fatty acids or derivatives thereof.

In some embodiments, medium chain solubilizing agents include, for example and without limitation, saturated medium chain fatty acids or derivatives of saturated medium chain fatty acids: caproic acid (C6), enanthic acid (C7), caprylic acid (C8), pelargonic acid (C9), capric acid (C10), undecylic acid (C11), lauric acid (C12), tridecylic acid (C13), or myristic acid (C14). In some embodiments, the solubilizing agent comprises oils made of these free medium chain fatty acids, oils of medium chain fatty acid esters of glycerin, propylene glycol, or ethylene glycol, or combinations thereof. These examples comprise predominantly saturated medium chain fatty acids (i.e., greater than 50% of the fatty acids are medium chain saturated fatty acids). In some embodiments, the solubilizing agent comprises predominantly C6 to C12 saturated fatty acids or derivatives of fatty acids.

In some embodiments, the solubilizing agent comprises one or more mono-, di-, or triglycerides or combinations thereof. Exemplary glycerin based solubilizing agents include MIGLYOLs®, which are caprylic/capric triglycerides (SASOL Germany GMBH, Hamburg). MIGLYOLs® includes MIGLYOL® 810 (caprylic/capric triglyceride), MIGLYOL® 812 (caprylic/capric triglyceride), MIGLYOL® 816 (caprylic/capric triglyceride), and MIGLYOL® 829 (caprylic/capric/succinic triglyceride). Other caprylic/capric triglyceride solubilizing agents are likewise contemplated, including, for example: caproic/caprylic/capric/lauric triglycerides; caprylic/capric/linoleic triglycerides; or caprylic/capric/succinic triglycerides. Other exemplary caprylic/capric mono-, di-, or triglyceride solubilizing agents include CAPMULs® (ABITEC, Columbus, Ohio), including, but are not limited to, CAPMUL® MCM, CAPMUL® MCM C10, CAPMUL® MCM C8, CAPMUL® MCM C8 EP, and CAPMUL® 708 G. Other mono-, di-, and triglycerides of fractionated vegetable fatty acids, and combinations or derivatives thereof can be the solubilizing agent, according to embodiments. For example, the solubilizing agent can be 1,2,3-propanetriol (glycerol, glycerin, glycerine) esters of saturated coconut and palm kernel oil and derivatives thereof.

In some embodiments, the solubilizing agent comprises one or more esters of propylene glycol, polyethylene glycol, or combinations thereof. Exemplary propylene and polyethylene glycol based solubilizing agents include glyceryl mono- and di-caprylates; propylene glycol monocaprylate (e.g., CAPMUL® PG-8 or CAPMUL® PG-8 NF); propylene glycol monocaprate (e.g., CAPMUL® PG-10); propylene glycol monolaurate (e.g., CAPMUL® PG-12 EP/NF); propylene glycol mono- and dicaprylates; propylene glycol mono- and dicaprate; propylene glycol dicaprylate/dicaprate (e.g., MIGLYOL® 840); propylene glycol dilaurate (e.g., CAPMUL® PG-2L EP/NF); diethylene glycol mono ester (e.g., TRANSCUTOL®,2-(2-Ethoxyethoxy)ethanol, GATTEFOSSÉ SAS, Saint-Priest, France); and diethylene glycol monoethyl ether.

In some embodiments, commercially available fatty acid glycerol and glycol ester solubilizing agents are prepared from natural oils and therefore may comprise components in addition to the fatty acid esters that predominantly comprise and characterize the solubilizing agent. Such other components may be, e.g., other fatty acid mono-, di-, and triglycerides, fatty acid mono- and diester ethylene or propylene glycols, free glycerols or glycols, or free fatty acids. For example, the Technical Data Sheet by ABITEC for CAPMUL® MCM C8 describes CAPMUL® MCM C8 as being composed of mono- and diglycerides of medium chain fatty acids (mainly caprylic) and describes the alkyl content as ≤1% C6, ≥95% C8, ≤5% C10, and ≤1.5% C12 and higher. By way of further example, MIGLYOL® 812 is generally described as a C8-C10 triglyceride because the fatty acid composition is at least about 80% caprylic (C8) acid and capric (C10) acid. However, it can also comprise small amounts of other fatty acids, e.g., less than about 5% of caproic (C6) acid, lauric (C12) acid, and myristic (C14) acid.

Any suitable amount of medium-chain oil can be used in the compositions disclosed herein. In some embodiments, the pharmaceutical composition comprises about 20% to about 85% solubilizing agent by weight, e.g., about 60% to about 85% solubilizing agent by weight. In some embodiments, the composition comprises progesterone, e.g., dissolved and micronized, from about 20 to about 50 wt %, e.g., about 30 to about 35 wt %. In some embodiments, the composition comprises estradiol from about 0.1 to about 0.8 wt %, e.g., about 0.15 to about 0.40 wt %.

Surfactants

In some embodiments, the pharmaceutical composition further comprises one or more non-ionic or ionic surfactants. In some embodiments, the non-ionic surfactant is selected from one or more of glycerol and polyethylene glycol esters of medium chain fatty acids or long chain fatty acids, for example, lauroyl macrogol-32 glycerides or lauroyl polyoxyl-32 glycerides, commercially available as GELUCIRE®, including, for example, GELUCIRE® 39/01 (glycerol esters of saturated C12-C18 fatty acids); GELUCIRE® 43/01 (hard fat NF/JPE); GELUCIRE® 44/14 (lauroyl macrogol-32 glycerides EP, lauroyl polyoxyl-32 glycerides NF, lauroyl polyoxylglycerides (USA FDA IIG)); and GELUCIRE® 50/13 (stearoyl macrogol-32 glycerides EP, stearoyl polyoxyl-32 glycerides NF, stearoyl polyoxylglycerides (USA FDA IIG)).

In some embodiments, non-ionic surfactants comprise combinations of mono- and di-propylene and ethylene glycols and mono-, di-, and triglyceride combinations. For example, in some embodiments, polyethylene glycol glyceride (GELUCIRE®, GATTEFOSSÉ SAS, Saint-Priest, France) can be used herein as the surfactant. For example, GELUCIRE® 44/14 (PEG-32 glyceryl laurate EP), a medium chain fatty acid esters of polyethylene glycol, is a polyethylene glycol glyceride composed of mono-, di- and triglycerides and mono- and diesters of polyethylene glycol.

In some embodiments, non-ionic surfactants include, for example and without limitation: one or more of oleic acid, linoleic acid, palmitic acid, and stearic acid. In some embodiments, non-ionic surfactants comprise polyethylene sorbitol esters, including polysorbate 80, which is commercially available under the trademark TWEEN 80® (Sigma Aldrich, St. Louis, Mo.). Polysorbate 80 comprises approximately 60%-70% oleic acid with the remainder comprising primarily linoleic acids, palmitic acids, and stearic acids.

In some embodiments, non-ionic surfactants include PEG-6 palmitostearate and ethylene glycol palmitostearate, which are available commercially as TEFOSE® 63 (GATTEFOSSÉ SAS, Saint-Priest, France). which can be used with, for example, CAPMUL® MCM having ratios of MCM to TEFOSE® 63 of, for example, 8:2 or 9:1. Other exemplary solubilizing agents/non-ionic surfactants combinations include, without limitation: MIGLYOL® 812:GELUCIRE 50/13 or MIGLYOL® 812:TEFOSE® 63.

A non-ionic or ionic surfactant may be used at concentrations greater than about 0.01%, for example at a concentration of about 0.01%-10.0%, about 0.1% to 10.0%, or about 1% to 10.0%. In some embodiments, the pharmaceutical composition comprises about 10.0% surfactant by weight. In some embodiments, the pharmaceutical composition comprises about 0.1% to about 5.0% surfactant by weight, e.g., about 1.0 wt %.

Other Excipients

In some embodiments, the pharmaceutical composition further comprises one more other excipients, such as but not limited to colorants, flavoring agents, preservatives, and taste-masking agents. The choice of excipients will, to a large extent, depend on factors such as the particular mode of administration, the effect of the excipients on solubility and stability, and the nature of the dosage form. Colorants, for example, may comprise about 0.1% to about 2% by weight. Preservatives may comprise methyl and propyl paraben, for example, in a ratio of about 10:1, and at a proportion of about 0.005% and 0.05% by weight.

Generally, the solubilizing agents, surfactants, and excipients used in the pharmaceutical compositions described herein are non-toxic, pharmaceutically acceptable, compatible with each other, and maintain stability of the pharmaceutical composition and the various components with respect to each other. Additionally, the combination of various components that comprise the pharmaceutical compositions will maintain will result in the desired therapeutic effect when administered to a subject.

Formulation

In some embodiments, combinations of solubilizing agents (e.g., two or more oils) or combinations of one or more solubilizing agents and one or more surfactants are used to form estradiol and progesterone compositions. Various ratios of these solubilizing agents or solubilizing agents and surfactants can be used. For example, CAPMUL® MCM and a non-ionic surfactant, e.g., GELUCIRE® 44/14 (lauroyl macrogol-32 glycerides EP; lauroyl polyoxyl-32 glycerides NF; lauroyl polyoxylglycerides (USA FDA IIG)), can be used at ratios of about 99:1 to about 2:1, including, for example and without limitation: 60:40, 65:35, 70:30, 75:25, 80:10, 80:15, 85:20, 90:10, and 98:1. As another example, CAPMUL® MCM and a non-ionic surfactant, e.g., TEFOSE® 63, can be used as rations of about 8:2 or 9:1. Other exemplary solubilizing agent/surfactant combinations include, without limitation: MIGLYOL® 812:GELUCIRE® 50/13 or MIGLYOL® 812:TEFOSE® 63. The ratios of oil (e.g., medium chain fatty acid esters of monoglycerides and diglycerides) to non-ionic surfactant can be significantly higher. For example, CAPMUL® MCM and GELUCIRE® can be used in ratios of up to about 65:1, e.g., 8:1, 22:1, 49:1, 65:1 and 66:1. Thus, useful ratios can be 8:1 or greater, e.g., 60 to 70:1.

In some embodiments, estradiol or progesterone is soluble in the solubilizing agent at room temperature, although it may be desirable to warm certain solubilizing agents. For example, when the formulation comprises medium chain fatty acid mono- and diglycerides (e.g., CAPMUL® MCM) and polyethylene glycol glycerides (e.g., GELUCIRE®) as a surfactant, the oil or the surfactant can be warmed up, e.g., to about 65° C. for the surfactant and less for the oil, to facilitate mixing of the oil and surfactant. The estradiol can be added at this temperature, or at lower temperatures as the mixture cools, e.g., about 40° C. or about 30° C., or even after the mixture has cooled to room temperature. The progesterone can also be added as the mixture cools, e.g., to below about 40° C. or to below about 30° C., or after the mixture has cooled to room temperature.

As a non-limiting example, a composition of this disclosure comprises solubilized estradiol; progesterone, at least 30% (e.g., at least about 30%, about 40%, about 50%, about 60%, about 70%, about 75%, about 80%, about 85%, or more) of the progesterone being solubilized (the balance being micronized as discussed elsewhere herein); and a solubilizing agent that is an oil, wherein the oil comprises medium chain fatty acid mono-, di-, or triglycerides, with or without a surfactant. In certain embodiments, a specification for progesterone is set at >80% solubilized, <20% micronized or >85% solubilized, <15% micronized. Specific examples of such illustrative embodiments, with CAPMUL® MCM NF (glyceryl caprylate/caprate) as a solubilizing agent and GELUCIRE® 44/14 (lauroyl polyoxyglyceride) as a surfactant, in which at least about 85% of the progesterone can be solubilized, include, e.g., the following five formulations A-E:

TABLE 1
Pharmaceutical Composition A - progesterone
50 mg/estradiol 0.25 mg

| | Amount | Qty/Capsule |
|---|---|---|
| Ingredient | (% w/w) | (mg) |
| Progesterone, USP, | 33.33 | 50.00 |
| micronized | | |
| Estradiol Hemihydrate | 0.17 | 0.26 |
| CAPMUL ® MCM, NF | 65.49 | 98.24 |
| GELUCIRE ® 44/14, NF | 1.00 | 1.50 |
| Total | 100.00 | 150.00 |

TABLE 2
Pharmaceutical Composition B - progesterone
50 mg/estradiol 0.5 mg

| | Amount | Qty/Capsule |
|---|---|---|
| Ingredient | (% w/w) | (mg) |
| Progesterone, USP, | 33.33 | 50.00 |
| micronized | | |
| Estradiol Hemihydrate | 0.35 | 0.52 |
| CAPMUL ® MCM, NF | 65.32 | 97.98 |
| GELUCIRE ® 44/14, NF | 1.00 | 1.50 |
| Total | 100.00 | 150.00 |

TABLE 3
Pharmaceutical Composition C - progesterone
100 mg/estradiol 0.5 mg

| | Amount | Qty/Capsule |
|---|---|---|
| Ingredient | (% w/w) | (mg) |
| Progesterone, USP, | 33.33 | 100.00 |
| micronized | | |
| Estradiol Hemihydrate | 0.17 | 0.52 |
| CAPMUL ® MCM, NF | 65.49 | 196.48 |
| GELUCIRE ® 44/14, NF | 1.00 | 3.00 |
| Total | 100.00 | 300.00 |

TABLE 4
Pharmaceutical Composition D - progesterone
100 mg/estradiol 1 mg

| | Amount | Qty/Capsule |
|---|---|---|
| Ingredient | (% w/w) | (mg) |
| Progesterone, USP, | 33.33 | 100.00 |
| micronized | | |
| Estradiol Hemihydrate | 0.34 | 1.03 |
| CAPMUL ® MCM, NF | 65.32 | 195.97 |
| GELUCIRE ® 44/14, NF | 1.00 | 3.00 |
| Total | 100.00 | 300.00 |

TABLE 5
Pharmaceutical Composition E - progesterone
200 mg/estradiol 2 mg

| | Amount | Qty/Capsule |
|---|---|---|
| Ingredient | (% w/w) | (mg) |
| Progesterone, USP, | 33.33 | 200.00 |
| micronized | | |
| Estradiol Hemihydrate | 0.34 | 2.06 |
| CAPMUL ® MCM, NF | 65.32 | 391.94 |
| GELUCIRE ® 44/14, NF | 1.00 | 6.00 |
| Total | 100.00 | 600.00 |

*Note: 1.00 mg Estradiol is equivalent to 1.03 mg Estradiol Hemihydrate

In general terms, the above formulations comprise 30 to 35 wt % progesterone, 0.1 to 0.4 wt % estradiol (or estradiol hemihydrate), 55 to 75 wt % of an oil that is predominantly medium chain fatty acid mono-, di-, or triglycerides, such as CAPMUL® MCM, and 0.5 to 10 wt % of a non-ionic surfactant, such as GELUCIRE® 44/14. The above formulations may be modified to comprise excipients, e.g., gelatin such as Gelatin 200 Bloom, glycerin, coloring agents such as Opatint red and white, and, optionally, MIGLYOL® 812.

Estradiol solubilization helps ensure high content uniformity and enhanced stability. Fully solubilized progesterone formulations or partially solubilized progesterone formulations in which at least about 50% of the progesterone, e.g., at least about 50%, 60%, 70%, 75%, 80%, 85%, 90%, 95% or more, is solubilized appear to provide improved PK-related properties.

### Pharmacokinetic Parameters of Estradiol and Progesterone Compositions

The pharmaceutical compositions of this disclosure can be formulated to provide desirable pharmacokinetic parameters in a subject (e.g., a female subject) to whom the composition is administered. In some embodiments, a pharmaceutical composition as described herein produces desirable pharmacokinetic parameters for progesterone in the subject. In some embodiments, a pharmaceutical composition as described herein produces desirable pharmacokinetic parameters for estradiol in the subject. In some embodiments, a pharmaceutical composition as described herein produces desirable pharmacokinetic parameters for one or more metabolites of progesterone or estradiol in the subject, for example, estrone or total estrone.

Following the administration of a composition comprising progesterone and estradiol to a subject, the concentration and metabolism of progesterone or estradiol can be measured in a sample (e.g., a blood, serum, or plasma sample) from the subject. Progesterone is metabolized to pregnanediols and pregnanolones, which are then conjugated to glucuronide and sulfate metabolites that are excreted or further recycled. Estradiol is converted reversibly to estrone, and both estradiol and estrone can be converted to the metabolite estriol. In postmenopausal women, a significant proportion of circulating estrogens exist as sulfate conjugates, especially estrone sulfate. Thus, estrone can be measured with respect to “estrone” amounts (excluding conjugates such as estrone sulfate) and “total estrone” amounts (including both free, or unconjugated, estrone and conjugated estrone such as estrone sulfate).

The pharmaceutical compositions of this disclosure can be characterized for one or more pharmacokinetic parameters of progesterone, estradiol, or a metabolite thereof following administration of the composition to a subject or to a population of subjects. These pharmacokinetic parameters include steady state concentration, AUC, Cₘₐₓ, and Tₘₐₓ. Steady state concentration is well understood in the art as the concentration (e.g., blood, plasma, or serum concentration) of an active pharmaceutical ingredient (e.g., progesterone or estradiol) at the time when the rate of intake of the active pharmaceutical ingredient is in equilibrium with the rate of elimination of the active pharmaceutical ingredient. AUC is a determination of the area under the curve (AUC) plotting the blood, serum, or plasma concentration of drug along the ordinate (Y-axis) against time along the abscissa (X-axis). AUCs are well understood, frequently used tools in the pharmaceutical arts and have been extensively described. Cₘₐₓ is well understood in the art as an abbreviation for the maximum drug concentration in blood, serum, or plasma of a subject. Tₘₐₓ is well understood in the art as an abbreviation for the time to maximum drug concentration in blood, serum, or plasma of a subject.

In some embodiments, one or more pharmacokinetic parameters, e.g., steady state concentration, AUC, Cₘₐₓ, or Tₘₐₓ, is measured for estradiol. In some embodiments, one or more pharmacokinetic parameters, e.g., steady state concentration, AUC, Cₘₐₓ, or Tₘₐₓ, is measured for progesterone. In some embodiments, one or more pharmacokinetic parameters, e.g., steady state concentration, AUC, Cₘₐₓ, or Tₘₐₓ, is measured for estrone. In some embodiments, one or more pharmacokinetic parameters, e.g., steady state concentration, AUC, Cₘₐₓ, or Tₘₐₓ, is measured for total estrone.

Any of a variety of methods can be used for measuring the levels of progesterone, estradiol, estrone, or total estrone in a sample, including immunoassays, mass spectrometry (MS), high performance liquid chromatography (HPLC) with ultraviolet fluorescent detection, liquid chromatography in conjunction with mass spectrometry (LC-MS), tandem mass spectrometry (MS/MS), and liquid chromatography-tandem mass spectrometry (LC-MS/MS). In some embodiments, the levels of progesterone, estradiol, estrone, or total estrone are measured using a validated LC-MS/MS method. Methods of measuring hormone levels are well described in the literature.

The levels of progesterone, estradiol, estrone, or total estrone can be measured in any biological sample, e.g. a tissue or fluid such as blood, serum, plasma, or urine. In some embodiments, the sample is blood or plasma.

In some embodiments, in the case of measuring steady state concentration, the levels of progesterone, estradiol, estrone, or total estrone are measured after a period of time of administering the composition comprising progesterone and estradiol to the subject. For example, in some embodiments, the steady state concentration of progesterone, estradiol, estrone, or total estrone is measured after at least 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of daily administration of the composition to the subject. In some embodiments, the steady state concentration of progesterone, estradiol, estrone, or total estrone is measured after at least 1 month, 2 months, 3 months, 4 months, 5 months, 6 months, 7 months, 8 months, 9 months, 10 months, 11 months, or 12 months of daily administration of the composition to the subject.

In some embodiments, in the case of measuring Cₘₐₓ, Tₘₐₓ, or AUC, the levels of progesterone, estradiol, estrone, or total estrone are measured about 0.0, 0.10, 0.20, 0.05, 0.30, 0.35, 0.40, 0.45, 0.5, 1, 2, 3, 4, 5, 6, 7, 8, 9, 10, 12, 15, 18, 21, 24, 27, 30, 33, 36, 39, 42, 45, or 48 hours after dosing, or any other appropriate time period that is common or useful in determining the levels of each of the hormones. In some embodiments, the levels of progesterone, estradiol, estrone, or total estrone are measured about 18 hours, about 24 hours, about 18-36 hours, about 20-30 hours, about 22-26 hours, about 24-36 hours, about 36 hours, about 36-48 hours, about 40-48 hours, or about 48 hours after administration of a single dose or a first dose. Generally, assays to determine the levels of progesterone, estradiol, estrone, or total estrone are measured one or more times every 5, 10, 15, 20, 30, 60, 120, 360, 480, 720, or 1440 minutes after administration, or combinations thereof (e.g., the first measurements are taken every 15 minutes for the first hour, followed by every 120 minutes thereafter). In embodiments, the timing of such measurements is designed to accurately measure Cₘₐₓ, Tₘₐₓ, or AUC. Timing can be adjusted based on the given circumstances (i.e., one formulation may cause a more rapid Cₘₐₓ, in which case the initial times would be clustered closer together, closer to time zero, or both to ensure accurate measurement of Cₘₐₓ, Tₘₐₓ, and AUC). In some embodiments, the Cₘₐₓ, Tₘₐₓ, or AUC values for progesterone, estradiol, estrone, or total estrone are measured following administration of a single dose of a pharmaceutical composition as described herein.

In some embodiments, the values for steady state concentration, Cₘₐₓ, Tₘₐₓ, or AUC represent a number of values taken from all the subjects in a patient population and are, therefore, mean values (e.g., arithmetic or geometric means) averaged over the entire population.

In some embodiments, oral administration of a pharmaceutical composition comprising estradiol, progesterone, and a medium chain solubilizing agent as described herein to a subject, or to a population of subjects, produces one or more steady state concentration, AUC, Cₘₐₓ, or Tₘₐₓ parameters, or one or more mean AUC, mean Cₘₐₓ, or mean Tₘₐₓ parameters, respectively, for estradiol, progesterone, estrone, or total estrone as described below.

AUC, Cₘₐₓ, and Tₘₐₓ Parameters (A)

In some embodiments, a pharmaceutical composition of this disclosure comprises estradiol at a dosage of about 0.25 mg and progesterone at a dosage of about 50 mg. In some embodiments, the pharmaceutical composition comprises the formulation of Formulation A in Table 1 above.

In some embodiments, administration of a composition comprising about 0.25 mg estradiol and about 50 mg progesterone to a subject produces, in a plasma or serum sample from the subject, one or more pharmacokinetic parameters selected from:
(i) a steady state estradiol concentration that is from 15.06 pg/mL to 18.50 pg/mL;
(ii) a steady state estrone concentration that is from 69.02 pg/mL to 73.43 pg/mL; and
(iii) a steady state progesterone concentration that is from 181.41 pg/mL to 247.17 pg/mL.

In some embodiments, administration of the composition to the subject produces a steady state estradiol concentration that is from 15.06 pg/mL to 18.50 pg/mL. In some embodiments, administration of the composition to the subject produces both of a steady state estradiol concentration that is from 15.06 pg/mL to 18.50 pg/mL and a steady state progesterone concentration that is from 181.41 pg/mL to 247.17 pg/mL. In some embodiments, administration of the composition to the subject produces both of a steady state estradiol concentration that is from 15.06 pg/mL to 18.50 pg/mL and a steady state estrone concentration that is from 69.02 pg/mL to 73.43 pg/mL. In some embodiments, administration of the composition to the subject produces each of: (i) a steady state estradiol concentration that is from 15.06 pg/mL to 18.50 pg/mL; (ii) a steady state estrone concentration that is from 69.02 pg/mL to 73.43 pg/mL; and (iii) a steady state progesterone concentration that is from 181.41 pg/mL to 247.17 pg/mL.

In some embodiments, administration of a composition comprising about 0.25 mg estradiol and about 50 mg progesterone to a subject produces, in a plasma sample from the subject, one or both parameters selected from:
(i) an AUC₍₀₋ₜ₎ for estradiol that is from 140.3733 pg·hr/ml to 219.3333 pg·hr/ml; or
(ii) a Cₘₐₓ for estradiol that is from 6.4790 pg/ml to 10.1235 pg/ml.

In some embodiments, administration of the composition to the subject produces both an AUC₍₀₋ₜ₎ for estradiol that is from 140.3733 pg·hr/ml to 219.3333 pg·hr/ml, and a Cₘₐₓ for estradiol that is from 6.4790 pg/ml to 10.1235 pg/ml.

In some embodiments, administration of the composition to the subject further produces, in a plasma sample from the subject, one or both parameters selected from:
(i) an AUC₍₀₋ₜ₎ for progesterone that is from 24.0174 ng·hr/ml to 37.5272 ng·hr/ml; or
(ii) a Cₘₐₓ for progesterone that is from 17.8444 ng/ml to 27.8819 ng/ml.

In some embodiments, administration of the composition to the subject produces both an AUC₍₀₋ₜ₎ for progesterone that is from 24.0174 ng·hr/ml to 37.5272 ng·hr/ml, and a Cₘₐₓ for progesterone that is from 17.8444 ng/ml to 27.8819 ng/ml.

In some embodiments, administration of the composition to the subject produces, in a plasma sample from the subject,
(i) an AUC₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one, two, three or more parameters selected from:
(i) an AUC₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 909.6091 pg·hr/ml to 1421.2642 pg·hr/ml;
(ii) a Cₘₐₓ for estrone that is from 42.6549 pg/ml to 66.6483 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 20.1752 ng·hr/ml to 31.5238 ng·hr/ml;
(ii) a Cₘₐₓ for total estrone that is from 3.5429 ng/ml to 5.5358 ng/ml; or
(iii) a Tₘₐₓ for total estrone that is from 2 hr to 3.1 hr.

In some embodiments, a pharmaceutical composition comprising about 0.25 mg estradiol and about 50 mg progesterone is administered to a population of subjects in need thereof, and mean parameters are determined for samples (e.g., blood, serum, or plasma samples) from the subjects administered the composition. Thus, in some embodiments, administration of the composition to a population of subject produces, in samples (e.g., plasma or serum samples) from the subjects, one or more of a mean steady state estradiol concentration that is from 15.06 pg/mL to 18.50 pg/mL; a mean AUC₍₀₋ₜ₎ for estradiol that is from 140.3733 pg·hr/ml to 219.3333 pg·hr/ml, a mean Cₘₐₓ for estradiol that is from 6.4790 pg/ml to 10.1235 pg/ml, and a mean Tₘₐₓ for estradiol that is from 7.2 hr to 11.3 hr. In some embodiments, administration of the composition to a population of subject produces, in samples from the subjects, one or more of a mean steady state progesterone concentration that is from 181.41 pg/mL to 247.17 pg/m, a mean AUC₍₀₋ₜ₎ for progesterone that is from 24.0174 ng·hr/ml to 37.5272 ng·hr/ml, a mean Cₘₐₓ for progesterone that is from 17.8444 ng/ml to 27.8819 ng/ml, and a mean Tₘₐₓ for progesterone that is from 2.4 hr to 3.8 hr. In some embodiments, administration of the composition to a population of subject produces, in samples from the subjects, one or more of a mean steady state estrone concentration that is from 69.02 pg/mL to 73.43 pg/mL, a mean AUC₍₀₋ₜ₎ for estrone that is from 909.6091 pg·hr/ml to 1421.2642 pg·hr/ml, a mean Cₘₐₓ for estrone that is from 42.6549 pg/ml to 66.6483 pg/ml, and a mean Tₘₐₓ for estrone that is from 4.4 hr to 6.9 hr. In some embodiments, administration of the composition to a population of subject produces, in samples from the subjects, one or more of a mean AUC₍₀₋ₜ₎ for total estrone that is from 20.1752 ng·hr/ml to 31.5238 ng·hr/ml, a mean Cₘₐₓ for total estrone that is from 3.5429 ng/ml to 5.5358 ng/ml, and a mean Tₘₐₓ for total estrone that is from 2 hr to 3.1 hr.

In some embodiments, methods of treating a subject with a pharmaceutical composition comprising estradiol and progesterone are provided. In some embodiments, the method comprises administering to the subject a pharmaceutical composition comprising about 0.25 mg estradiol and about 50 mg progesterone as described herein (e.g., a pharmaceutical composition having the formulation of Formulation A in Table 1 above), wherein administration of the pharmaceutical composition produces, in a sample (e.g., plasma or serum sample) from the subject, one or more parameters selected from: a steady state estradiol concentration that is from 15.06 pg/mL to 18.50 pg/mL; an AUC₍₀₋ₜ₎ for estradiol that is from 140.3733 pg·hr/ml to 219.3333 pg·hr/ml; a Cₘₐₓ for estradiol that is from 6.4790 pg/ml to 10.1235 pg/ml; a Tₘₐₓ for estradiol that is from 7.2 hr to 11.3 hr; a steady state progesterone concentration that is from 181.41 pg/mL to 247.17 pg/mL; an AUC₍₀₋ₜ₎ for progesterone that is from 24.0174 ng·hr/ml to 37.5272 ng·hr/ml; a Cₘₐₓ for progesterone that is from 17.8444 ng/ml to 27.8819 ng/ml; a Tₘₐₓ for progesterone that is from 2.4 hr to 3.8 hr; steady state estrone concentration that is from 69.02 pg/mL to 73.43 pg/mL; an AUC₍₀₋ₜ₎ for estrone that is from 909.6091 pg·hr/ml to 1421.2642 pg·hr/ml; a Cₘₐₓ for estrone that is from 42.6549 pg/ml to 66.6483 pg/ml; a Tₘₐₓ for estrone that is from 4.4 hr to 6.9 hr; an AUC₍₀₋ₜ₎ for total estrone that is from 20.1752 ng·hr/ml to 31.5238 ng·hr/ml; a Cₘₐₓ for total estrone that is from 3.5429 ng/ml to 5.5358 ng/ml; and a Tₘₐₓ for total estrone that is from 2 hr to 3.1 hr.

AUC, Cₘₐₓ, and Tₘₐₓ Parameters (B)

In some embodiments, a pharmaceutical composition of this disclosure comprises estradiol at a dosage of about 0.50 mg and progesterone at a dosage of about 50 mg. In some embodiments, the pharmaceutical composition comprises the formulation of Formulation B in Table 2 above.

In some embodiments, administration of a composition comprising about 0.50 mg estradiol and about 50 mg progesterone to a subject produces, in a plasma sample from the subject, one or more pharmacokinetic parameters selected from:
(i) a steady state estradiol concentration that is from 23.03 pg/mL to 27.37 pg/mL;
(ii) a steady state estrone concentration that is from 113.59 pg/mL to 132.08 pg/mL; and
(iii) a steady state progesterone concentration that is from 181.41 pg/mL to 247.17 pg/mL.

In some embodiments, administration of the composition to the subject produces a steady state estradiol concentration that is from 23.03 pg/mL to 27.37 pg/mL. In some embodiments, administration of the composition to the subject produces both of a steady state estradiol concentration that is from 23.03 pg/mL to 27.37 pg/mL and a steady state progesterone concentration that is from 181.41 pg/mL to 247.17 pg/mL. In some embodiments, administration of the composition to the subject produces both of a steady state estradiol concentration that is from 23.03 pg/mL to 27.37 pg/mL and a steady state estrone concentration that is from 113.59 pg/mL to 132.08 pg/mL. In some embodiments, administration of the composition to the subject produces each of: (i) a steady state estradiol concentration that is from 23.03 pg/mL to 27.37 pg/mL; (ii) a steady state estrone concentration that is from 113.59 pg/mL to 132.08 pg/mL; and (iii) a steady state progesterone concentration that is from 181.41 pg/mL to 247.17 pg/mL.

In some embodiments, administration of a composition comprising about 0.50 mg estradiol and about 50 mg progesterone to a subject produces, in a plasma sample from the subject, one or both parameters selected from:
(i) an AUC₍₀₋ₜ₎ for estradiol that is from 280.7467 pg·hr/ml to 438.6667 pg·hr/ml; or
(ii) a Cₘₐₓ for estradiol that is from 12.9580 pg/ml to 20.2469 pg/ml.

In some embodiments, administration of the composition to the subject produces both an AUC₍₀₋ₜ₎ for estradiol that is from 280.7467 pg·hr/ml to 438.6667 pg·hr/ml, and a Cₘₐₓ for estradiol that is from 12.9580 pg/ml to 20.2469 pg/ml.

In some embodiments, administration of the composition to the subject further produces, in a plasma sample from the subject, one or both parameters selected from:
(i) an AUC₍₀₋ₜ₎ for progesterone that is from 24.0174 ng·hr/ml to 37.5272 ng·hr/ml; or
(ii) a Cₘₐₓ for progesterone that is from 17.8444 ng/ml to 27.8819 ng/ml.

In some embodiments, administration of the composition to the subject produces both an (AUC)₍₀₋ₜ₎ for progesterone that is from 24.0174 ng·hr/ml to 37.5272 ng·hr/ml, and a Cₘₐₓ for progesterone that is from 17.8444 ng/ml to 27.8819 ng/ml.

In some embodiments, administration of the composition to the subject produces, in a plasma sample from the subject,
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one, two, three or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 1819.2181 pg·hr/ml to 2842.5283 pg·hr/ml;
(ii) a Cₘₐₓ for estrone that is from 85.3098 pg/ml to 133.2966 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 40.3505 ng·hr/ml to 63.0476 ng·hr/ml;
(ii) a Cₘₐₓ for total estrone that is from 7.0858 ng/ml to 11.0715 ng/ml; or
(iii) a Tₘₐₓ for total estrone that is from 2 hr to 3.1 hr.

In some embodiments, a pharmaceutical composition comprising about 0.50 mg estradiol and about 50 mg progesterone is administered to a population of subjects in need thereof, and mean parameters are determined for samples (e.g., blood, serum, or plasma samples) from the subjects administered the composition. Thus, in some embodiments, administration of the composition to a population of subject produces, in samples (e.g., serum or plasma samples) from the subjects, one or more of a mean steady state estradiol concentration that is from 23.03 pg/mL to 27.37 pg/mL; a mean AUC₍₀₋ₜ₎ for estradiol that is from 280.7467 pg·hr/ml to 438.6667 pg·hr/ml, a mean Cₘₐₓ for estradiol that is from 12.9580 pg/ml to 20.2469 pg/ml, and a mean Tₘₐₓ for estradiol that is from 7.2 hr to 11.3 hr. In some embodiments, administration of the composition to a population of subject produces, in samples from the subjects, one or more of a mean steady state progesterone concentration that is from 181.41 pg/mL to 247.17 pg/mL; a mean AUC₍₀₋ₜ₎ for progesterone that is from 24.0174 ng·hr/ml to 37.5272 ng·hr/ml, a mean Cₘₐₓ for progesterone that is from 17.8444 ng/ml to 27.8819 ng/ml, and a mean Tₘₐₓ for progesterone that is from 2.4 hr to 3.8 hr. In some embodiments, administration of the composition to a population of subject produces, in samples from the subjects, one or more of a mean steady state estrone concentration that is from 113.59 pg/mL to 132.08 pg/mL; a mean AUC₍₀₋ₜ₎ for estrone that is from 1819.2181 pg·hr/ml to 2842.5283 pg·hr/ml, a mean Cₘₐₓ for estrone that is from 85.3098 pg/ml to 133.2966 pg/ml, and a mean Tₘₐₓ for estrone that is from 4.4 hr to 6.9 hr. In some embodiments, administration of the composition to a population of subject produces, in samples from the subjects, one or more of a mean AUC₍₀₋ₜ₎ for total estrone that is from 40.3505 ng·hr/ml to 63.0476 ng·hr/ml, a mean Cₘₐₓ for total estrone that is from 7.0858 ng/ml to 11.0715 ng/ml, and a mean Tₘₐₓ for total estrone that is from 2 hr to 3.1 hr.

In some embodiments, methods of treating a subject with a pharmaceutical composition comprising estradiol and progesterone are provided. In some embodiments, the method comprises administering to the subject a pharmaceutical composition comprising about 0.50 mg estradiol and about 50 mg progesterone as described herein (e.g., a pharmaceutical composition having the formulation of Formulation B in Table 2 above), wherein administration of the pharmaceutical composition produces, in a serum or plasma sample from the subject, one or more parameters selected from: a steady state estradiol concentration that is from 23.03 pg/mL to 27.37 pg/mL; an AUC₍₀₋ₜ₎ for estradiol that is from 280.7467 pg·hr/ml to 438.6667 pg·hr/ml; a Cₘₐₓ for estradiol that is from 12.9580 pg/ml to 20.2469 pg/ml; a Tₘₐₓ for estradiol that is from 7.2 hr to 11.3 hr; a steady state progesterone concentration that is from 181.41 pg/mL to 247.17 pg/mL; an AUC₍₀₋ₜ₎ for progesterone that is from 24.0174 ng·hr/ml to 37.5272 ng·hr/ml; a Cₘₐₓ for progesterone that is from 17.8444 ng/ml to 27.8819 ng/ml; a Tₘₐₓ for progesterone that is from 2.4 hr to 3.8 hr; a steady state estrone concentration that is from 113.59 pg/mL to 132.08 pg/mL; an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; a Tₘₐₓ for estrone that is from 4.4 hr to 6.9 hr; an AUC₍₀₋ₜ₎ for total estrone that is from 40.3505 ng·hr/ml to 63.0476 ng·hr/ml; a Cₘₐₓ for total estrone that is from 7.0858 ng/ml to 11.0715 ng/ml; and a Tₘₐₓ for total estrone that is from 2 hr to 3.1 hr.

AUC, Cₘₐₓ, and Tₘₐₓ Parameters (C)

In some embodiments, a pharmaceutical composition of this disclosure comprises estradiol at a dosage of about 0.50 mg and progesterone at a dosage of about 100 mg. In some embodiments, the pharmaceutical composition comprises the formulation of Formulation C in Table 3 above.

In some embodiments, administration of a composition comprising about 0.50 mg estradiol and about 100 mg progesterone to a subject produces, in a plasma sample from the subject, one or more pharmacokinetic parameters selected from:
(i) a steady state estradiol concentration that is from 23.03 pg/mL to 27.37 pg/mL;
(ii) a steady state estrone concentration that is from 113.59 pg/mL to 132.08 pg/mL; and
(iii) a steady state progesterone concentration that is from 386.53 pg/mL to 547.83 pg/mL.

In some embodiments, administration of the composition to the subject produces a steady state estradiol concentration that is from 23.03 pg/mL to 27.37 pg/mL. In some embodiments, administration of the composition to the subject produces both of a steady state estradiol concentration that is from 23.03 pg/mL to 27.37 pg/mL and a steady state progesterone concentration that is from 386.53 pg/mL to 547.83 pg/mL. In some embodiments, administration of the composition to the subject produces both of a steady state estradiol concentration that is from 23.03 pg/mL to 27.37 pg/mL and a steady state estrone concentration that is from 113.59 pg/mL to 132.08 pg/mL. In some embodiments, administration of the composition to the subject produces each of: (i) a steady state estradiol concentration that is from 23.03 pg/mL to 27.37 pg/mL; (ii) a steady state estrone concentration that is from 113.59 pg/mL to 132.08 pg/mL; and (iii) a steady state progesterone concentration that is from 386.53 pg/mL to 547.83 pg/mL.

In some embodiments, administration of a composition comprising about 0.50 mg estradiol and about 100 mg progesterone to a subject produces, in a plasma sample from the subject, one or both parameters selected from:
(i) an AUC₍₀₋ₜ₎ for estradiol that is from 280.7467 pg·hr/ml to 438.6667 pg·hr/ml; or
(ii) a Cₘₐₓ for estradiol that is from 12.9580 pg/ml to 20.2469 pg/ml.

In some embodiments, administration of the composition to the subject produces both an AUC₍₀₋ₜ₎ for estradiol that is from 280.7467 pg·hr/ml to 438.6667 pg·hr/ml, and a Cₘₐₓ for estradiol that is from 12.9580 pg/ml to 20.2469 pg/ml.

In some embodiments, administration of the composition to the subject further produces, in a plasma sample from the subject, one or both parameters selected from:
(i) an AUC₍₀₋ₜ₎ for progesterone that is from 48.0348 ng·hr/ml to 75.0543 ng·hr/ml; or
(ii) a Cₘₐₓ for progesterone that is from 35.6889 ng/ml to 55.7639 ng/ml.

In some embodiments, administration of the composition to the subject produces both an AUC₍₀₋ₜ₎ for progesterone that is from 48.0348 ng·hr/ml to 75.0543 ng·hr/ml, and a Cₘₐₓ for progesterone that is from 35.6889 ng/ml to 55.7639 ng/ml.

In some embodiments, administration of the composition to the subject produces, in a plasma sample from the subject,
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one, two, three or more parameters selected from:
(i) an AUC₍₀₋ₜ₎ for estrone that is from 1819.2181 pg·hr/ml to 2842.5283 pg·hr/ml;
(ii) a Cₘₐₓ for estrone that is from 85.3098 pg/ml to 133.2966 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 40.3505 ng·hr/ml to 63.0476 ng·hr/ml;
(ii) a Cₘₐₓ for total estrone that is from 7.0858 ng/ml to 11.0715 ng/ml; or
(iii) a Tₘₐₓ for total estrone that is from 2 hr to 3.1 hr.

In some embodiments, a pharmaceutical composition comprising about 0.50 mg estradiol and about 100 mg progesterone is administered to a population of subjects in need thereof, and mean parameters are determined for samples (e.g., blood, serum, and plasma samples) from the subjects administered the composition. Thus, in some embodiments, administration of the composition to a population of subject produces, in samples (e.g., serum or plasma samples) from the subjects, one or more of a mean steady state estradiol concentration that is from 23.03 pg/mL to 27.37 pg/mL; a mean AUC₍₀₋ₜ₎ for estradiol that is from 280.7467 pg·hr/ml to 438.6667 pg·hr/ml, a mean Cₘₐₓ for estradiol that is from 12.9580 pg/ml to 20.2469 pg/ml, and a mean Tₘₐₓ for estradiol that is from 7.2 hr to 11.3 hr. In some embodiments, administration of the composition to a population of subject produces, in samples from the subjects, one or more of a mean steady state progesterone concentration that is from 386.53 pg/mL to 547.83 pg/mL; a mean AUC₍₀₋ₜ₎ for progesterone that is from 48.0348 ng·hr/ml to 75.0543 ng·hr/ml, a mean Cₘₐₓ for progesterone that is from 35.6889 ng/ml to 55.7639 ng/ml, and a mean Tₘₐₓ for progesterone that is from 2.4 hr to 3.8 hr. In some embodiments, administration of the composition to a population of subject produces, in samples from the subjects, one or more of a mean steady state estrone concentration that is from 113.59 pg/mL to 132.08 pg/mL; a mean AUC₍₀₋ₜ₎ for estrone that is from 1819.2181 pg·hr/ml to 2842.5283 pg·hr/ml, a mean Cₘₐₓ for estrone that is from 85.3098 pg/ml to 133.2966 pg/ml, and a mean Tₘₐₓ for estrone that is from 4.4 hr to 6.9 hr. In some embodiments, administration of the composition to a population of subject produces, in samples from the subjects, one or more of a mean AUC₍₀₋ₜ₎ for total estrone that is from 40.3505 ng·hr/ml to 63.0476 ng·hr/ml, a mean Cₘₐₓ for total estrone that is from 7.0858 ng/ml to 11.0715 ng/ml, and a mean Tₘₐₓ for total estrone that is from 2 hr to 3.1 hr.

In some embodiments, method of treating a subject with a pharmaceutical composition comprising estradiol and progesterone are provided. In some embodiments, the method comprises administering to the subject a pharmaceutical composition comprising about 0.50 mg estradiol and about 100 mg progesterone as described herein (e.g., a pharmaceutical composition having the formulation of Formulation C in Table 3 above), wherein administration of the pharmaceutical composition produces, in a serum or plasma sample from the subject, one or more parameters selected from: a steady state estradiol concentration that is from 23.03 pg/mL to 27.37 pg/mL; an AUC₍₀₋ₜ₎ for estradiol that is from 280.7467 pg·hr/ml to 438.6667 pg·hr/ml; a Cₘₐₓ for estradiol that is from 12.9580 pg/ml to 20.2469 pg/ml; a Tₘₐₓ for estradiol that is from 7.2 hr to 11.3 hr; a steady state progesterone concentration that is from 386.53 pg/mL to 547.83 pg/mL; an AUC₍₀₋ₜ₎ for progesterone that is from 48.0348 ng·hr/ml to 75.0543 ng·hr/ml; a Cₘₐₓ for progesterone that is from 35.6889 ng/ml to 55.7639 ng/ml; a Tₘₐₓ for progesterone that is from 2.4 hr to 3.8 hr; a steady state estrone concentration that is from 113.59 pg/mL to 132.08 pg/mL; an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; a Tₘₐₓ for estrone that is from 4.4 hr to 6.9 hr; an AUC₍₀₋ₜ₎ for total estrone that is from 40.3505 ng·hr/ml to 63.0476 ng·hr/ml; a Cₘₐₓ for total estrone that is from 7.0858 ng/ml to 11.0715 ng/ml; and a Tₘₐₓ for total estrone that is from 2 hr to 3.1 hr.

AUC, Cₘₐₓ, and Tₘₐₓ Parameters (D)

In some embodiments, a pharmaceutical composition of this disclosure comprises estradiol at a dosage of about 1 mg and progesterone at a dosage of about 100 mg. In some embodiments, the pharmaceutical composition comprises the formulation of Formulation D in Table 4 above.

In some embodiments, administration of a composition comprising about 1 mg estradiol and about 100 mg progesterone to a subject produces, in a plasma sample from the subject, one or more pharmacokinetic parameters selected from:
(i) a steady state estradiol concentration that is from 42.29 pg/mL to 45.58 pg/mL;
(ii) a steady state estrone concentration that is from 213.79 pg/mL to 241.57 pg/mL; and
(iii) a steady state progesterone concentration that is from 386.53 pg/mL to 547.83 pg/mL.

In some embodiments, administration of the composition to the subject produces a steady state estradiol concentration that is from 42.29 pg/mL to 45.58 pg/mL. In some embodiments, administration of the composition to the subject produces both of a steady state estradiol concentration that is from 42.29 pg/mL to 45.58 pg/mL and a steady state progesterone concentration that is from 386.53 pg/mL to 547.83 pg/mL. In some embodiments, administration of the composition to the subject produces both of a steady state estradiol concentration that is from 42.29 pg/mL to 45.58 pg/mL and a steady state estrone concentration that is from 213.79 pg/mL to 241.57 pg/mL. In some embodiments, administration of the composition to the subject produces each of: (i) a steady state estradiol concentration that is from 42.29 pg/mL to 45.58 pg/mL; (ii) a steady state estrone concentration that is from 213.79 pg/mL to 241.57 pg/mL; and (iii) a steady state progesterone concentration that is from 386.53 pg/mL to 547.83 pg/mL.

In some embodiments, administration of a composition comprising about 1 mg estradiol and about 100 mg progesterone to a subject produces, in a plasma sample from the subject, one or both parameters selected from:
(i) an AUC₍₀₋ₜ₎ for estradiol that is from 561.4933 pg·hr/ml to 877.3333 pg·hr/ml; or
(ii) a Cₘₐₓ for estradiol that is from 25.9161 pg/ml to 40.4939 pg/ml.

In some embodiments, administration of the composition to the subject produces both an AUC₍₀₋ₜ₎ for estradiol that is from 561.4933 pg·hr/ml to 877.3333 pg·hr/ml, and a Cₘₐₓ for estradiol that is from 25.9161 pg/ml to 40.4939 pg/ml.

In some embodiments, administration of the composition to the subject further produces, in a plasma sample from the subject, one or both parameters selected from:
(i) an AUC₍₀₋ₜ₎ for progesterone that is from 48.0348 ng·hr/ml to 75.0543 ng·hr/ml; or
(ii) a Cₘₐₓ for progesterone that is from 35.6889 ng/ml to 55.7639 ng/ml.

In some embodiments, administration of the composition to the subject produces both an AUC₍₀₋ₜ₎ for progesterone that is from 48.0348 ng·hr/ml to 75.0543 ng·hr/ml, and a Cₘₐₓ for progesterone that is from 35.6889 ng/ml to 55.7639 ng/ml.

In some embodiments, administration of the composition to the subject produces, in a plasma sample from the subject,
(i) an AUC₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the composition to the subject produces, in a plasma sample from the subject, one, two, three or more parameters selected from:
(i) an AUC₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 3638.4363 pg·hr/ml to 5685.0567 pg·hr/ml;
(ii) a Cₘₐₓ for estrone that is from 170.6197 pg/ml to 266.5933 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 80.7010 ng·hr/ml to 126.0953 ng·hr/ml;
(ii) a Cₘₐₓ for total estrone that is from 14.1716 ng/ml to 22/1431 ng/ml; or
(iii) a Tₘₐₓ for total estrone that is from 2 hr to 3.1 hr.

In some embodiments, a pharmaceutical composition comprising about 1 mg estradiol and about 100 mg progesterone is administered to a population of subjects in need thereof, and mean parameters are determined for samples (e.g., blood, serum, or plasma samples) from the subjects administered the composition. Thus, in some embodiments, administration of the composition to a population of subject produces, in samples (e.g., serum or plasma samples) from the subjects, one or more of a mean steady state estradiol concentration that is from 42.29 pg/mL to 45.58 pg/mL; a mean AUC₍₀₋ₜ₎ for estradiol that is from 561.4933 pg·hr/ml to 877.3333 pg·hr/ml, a mean Cₘₐₓ for estradiol that is from 25.9161 pg/ml to 40.4939 pg/ml, and a mean Tₘₐₓ for estradiol that is from 7.2 hr to 11.3 hr. In some embodiments, administration of the composition to a population of subject produces, in samples from the subjects, one or more of a mean steady state progesterone concentration that is from 386.53 pg/mL to 547.83 pg/mL; a mean AUC₍₀₋ₜ₎ for progesterone that is from 48.0348 ng·hr/ml to 75.0543 ng·hr/ml, a mean Cₘₐₓ for progesterone that is from 35.6889 ng/ml to 55.7639 ng/ml, and a mean Tₘₐₓ for progesterone that is from 2.4 hr to 3.8 hr. In some embodiments, administration of the composition to a population of subject produces, in samples from the subjects, one or more of a mean steady state estrone concentration that is from 213.79 pg/mL to 241.57 pg/mL; a mean AUC₍₀₋ₜ₎ for estrone that is from 3638.4363 pg·hr/ml to 5685.0567 pg·hr/ml, a mean Cₘₐₓ for estrone that is from 170.6197 pg/ml to 266.5933 pg/ml, and a mean Tₘₐₓ for estrone that is from 4.4 hr to 6.9 hr. In some embodiments, administration of the composition to a population of subject produces, in samples from the subjects, one or more of a mean AUC₍₀₋ₜ₎ for total estrone that is from 80.7010 ng·hr/ml to 126.0953 ng·hr/ml, a mean Cₘₐₓ for total estrone that is from 14.1716 ng/ml to 22/1431 ng/ml, and a mean Tₘₐₓ for total estrone that is from 2 hr to 3.1 hr.

In some embodiments, method of treating a subject with a pharmaceutical composition comprising estradiol and progesterone are provided. In some embodiments, the method comprises administering to the subject a pharmaceutical composition comprising about 1 mg estradiol and about 100 mg progesterone as described herein (e.g., a pharmaceutical composition having the formulation of Formulation D in Table 4 above), wherein administration of the pharmaceutical composition produces, in a serum or plasma sample from the subject, one or more parameters selected from: a steady state estradiol concentration that is from 42.29 pg/mL to 45.58 pg/mL; an AUC₍₀₋ₜ₎ for estradiol that is from 561.4933 pg·hr/ml to 877.3333 pg·hr/m; a Cₘₐₓ for estradiol that is from 25.9161 pg/ml to 40.4939 pg/ml; a Tₘₐₓ for estradiol that is from 7.2 hr to 11.3 hr; a steady state progesterone concentration that is from 386.53 pg/mL to 547.83 pg/mL; an AUC₍₀₋ₜ₎ for progesterone that is from 48.0348 ng·hr/ml to 75.0543 ng·hr/ml; a Cₘₐₓ for progesterone that is from 35.6889 ng/ml to 55.7639 ng/ml; a Tₘₐₓ for progesterone that is from 2.4 hr to 3.8 hr; a steady state estrone concentration that is from 213.79 pg/mL to 241.57 pg/mL; an AUC₍₀₋ₜ₎ for estrone that is from 3638.4363 pg·hr/ml to 5685.0567 pg·hr/ml; a Cₘₐₓ for estrone that is from 170.6197 pg/ml to 266.5933 pg/ml; a Tₘₐₓ for estrone that is from 4.4 hr to 6.9 hr; an AUC₍₀₋ₜ₎ for total estrone that is from 80.7010 ng·hr/ml to 126.0953 ng·hr/ml; a Cₘₐₓ for total estrone that is from 14.1716 ng/ml to 22/1431 ng/ml; and a Tₘₐₓ for total estrone that is from 2 hr to 3.1 hr.

AUC, Cₘₐₓ, and Tₘₐₓ Parameters (E)

In some embodiments, a pharmaceutical composition of this disclosure comprises estradiol at a dosage of about 2 mg and progesterone at a dosage of about 200 mg. In some embodiments, the pharmaceutical composition comprises the formulation of Formulation E in Table 5 above.

In some embodiments, administration of a pharmaceutical composition comprising about 2 mg estradiol and about 200 mg progesterone to a subject produces, in a plasma sample from the subject, one or both parameters selected from:
(i) an AUC₍₀₋ₜ₎ for estradiol that is from 1123 pg·h/ml to 1755 pg·h/ml; or
(ii) a Cₘₐₓ for estradiol that is from 52 pg/ml to 81 pg/ml.

In some embodiments, administration of the composition to the subject produces both an AUC₍₀₋ₜ₎ for estradiol that is from 1123 pg·h/ml to 1755 pg·h/ml, and a Cₘₐₓ for estradiol that is from 52 pg/ml to 81 pg/ml.

In some embodiments, administration of the composition to the subject further produces, in a plasma sample from the subject, one or both parameters selected from:
(i) an AUC₍₀₋ₜ₎ for progesterone that is from 96 ng·hr/ml to 150 ng·hr/ml; or
(ii) a Cₘₐₓ for progesterone that is from 71 ng/ml to 112 ng/ml.

In some embodiments, administration of the composition to the subject produces both an AUC₍₀₋ₜ₎ for progesterone that is from 96 ng·hr/ml to 150 ng·hr/ml, and a Cₘₐₓ for progesterone that is from 71 ng/ml to 112 ng/ml.

In some embodiments, administration of the composition to the subject produces, in a plasma sample from the subject,
(i) an AUC₍₀₋ₜ₎ for estradiol that is from 1123 pg·h/ml to 1755 pg·h/ml;
(ii) a Cₘₐₓ for estradiol that is from 52 pg/ml to 81 pg/ml;
(iii) an AUC₍₀₋ₜ₎ for progesterone that is from 96 ng·hr/ml to 150 ng·hr/ml; or
(iv) a Cₘₐₓ for progesterone that is from 71 ng/ml to 112 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one, two, three or more parameters selected from:
(i) an AUC₍₀₋ₜ₎ for estradiol that is from 1123 pg·h/ml to 1755 pg·h/ml;
(ii) a Cₘₐₓ for estradiol that is from 52 pg/ml to 81 pg/ml;
(iii) an AUC₍₀₋ₜ₎ for progesterone that is from 96 ng·hr/ml to 150 ng·hr/ml; or
(iv) a Cₘₐₓ for progesterone that is from 71 ng/ml to 112 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 7277 pg·hr/ml to 11370 pg·hr/ml;
(ii) a Cₘₐₓ for estrone that is from 341 pg/ml to 533 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 161 ng·h/ml to 252 ng·h/ml
(ii) a Cₘₐₓ for total estrone that is from 28 ng/ml to 44 ng/ml; or
(iii) a Tₘₐₓ for total estrone that is from 2 hr to 3.1 hr.

In some embodiments, a pharmaceutical composition comprising about 2 mg estradiol and about 200 mg progesterone is administered to a population of subjects in need thereof, and mean parameters are determined for samples (e.g., blood or plasma samples) from the subjects administered the composition. Thus, in some embodiments, administration of the composition to a population of subject produces, in plasma samples from the subjects, one or more of a mean AUC₍₀₋ₜ₎ for estradiol that is from 1123 pg·h/ml to 1755 pg·h/ml, a mean Cₘₐₓ for estradiol that is from 52 pg/ml to 81 pg/ml, and a mean Tₘₐₓ for estradiol that is from 7.2 hr to 11.3 hr. In some embodiments, administration of the composition to a population of subject produces, in plasma samples from the subjects, one or more of a mean AUC₍₀₋ₜ₎ for progesterone that is from 96 ng·hr/ml to 150 ng·hr/ml, a mean Cₘₐₓ for progesterone that is from 71 ng/ml to 112 ng/ml, and a mean Tₘₐₓ for progesterone that is from 2.4 hr to 3.8 hr. In some embodiments, administration of the composition to a population of subject produces, in plasma samples from the subjects, one or more of a mean AUC₍₀₋ₜ₎ for estrone that is from 7277 pg·hr/ml to 11370 pg·hr/ml, a mean Cₘₐₓ for estrone that is from 341 pg/ml to 533 pg/ml, and a mean Tₘₐₓ for estrone that is from 4.4 hr to 6.9 hr. In some embodiments, administration of the composition to a population of subject produces, in plasma samples from the subjects, one or more of a mean AUC₍₀₋ₜ₎ for total estrone that is from 161 ng·h/ml to 252 ng·h/ml, a mean Cₘₐₓ for total estrone that is from 28 ng/ml to 44 ng/ml, and a mean Tₘₐₓ for total estrone that is from 2 hr to 3.1 hr.

In some embodiments, method of treating a subject with a pharmaceutical composition comprising estradiol and progesterone are provided. In some embodiments, the method comprises administering to the subject a pharmaceutical composition comprising about 2 mg estradiol and about 200 mg progesterone as described herein (e.g., a pharmaceutical composition having the formulation of Formulation E in Table 5 above), wherein administration of the pharmaceutical composition produces, in a plasma sample from the subject, one or more parameters selected from: an AUC₍₀₋ₜ₎ for estradiol that is from 1123 pg·h/ml to 1755 pg·h/ml; a Cₘₐₓ for estradiol that is from 52 pg/ml to 81 pg/ml; a Tₘₐₓ for estradiol that is from 7.2 hr to 11.3 hr; an AUC₍₀₋ₜ₎ for progesterone that is from 96 ng·hr/ml to 150 ng·hr/ml; a Cₘₐₓ for progesterone that is from 71 ng/ml to 112 ng/ml; a Tₘₐₓ for progesterone that is from 2.4 hr to 3.8 hr; an AUC₍₀₋ₜ₎ for estrone that is from 7277 pg·hr/ml to 11370 pg·hr/ml; a Cₘₐₓ for estrone that is from 341 pg/ml to 533 pg/ml; a Tₘₐₓ for estrone that is from 4.4 hr to 6.9 hr; an AUC₍₀₋ₜ₎ for total estrone that is from 161 ng·h/ml to 252 ng·h/ml; a Cₘₐₓ for total estrone that is from 28 ng/ml to 44 ng/ml; and a Tₘₐₓ for total estrone that is from 2 hr to 3.1 hr.

In some embodiments, the method further comprises obtaining a sample from the subject (e.g., a blood or plasma sample) following administration of a single dose of the pharmaceutical composition (e.g., a pharmaceutical composition having the formulation of Formulation E in Table 5 above), and measuring one or more pharmacokinetic parameters selected from an AUC₍₀₋ₜ₎ for estradiol, a Cₘₐₓ for estradiol, an AUC₍₀₋ₜ₎ for progesterone, a Cₘₐₓ for progesterone, an AUC₍₀₋ₜ₎ for estrone, a Cₘₐₓ for estrone, an AUC₍₀₋ₜ₎ for total estrone, and a Cₘₐₓ for total estrone; wherein the presence of one or more of the following values is indicative of a therapeutically effective dose: an AUC₍₀₋ₜ₎ for estradiol that is from 1123 pg·h/ml to 1755 pg·h/ml; a Cₘₐₓ for estradiol that is from 52 pg/ml to 81 pg/ml; an AUC₍₀₋ₜ₎ for progesterone that is from 96 ng·hr/ml to 150 ng·hr/ml; a Cₘₐₓ for progesterone that is from 71 ng/ml to 112 ng/ml; an AUC₍₀₋ₜ₎ for estrone that is from 7277 pg·hr/ml to 11370 pg·hr/ml; a Cₘₐₓ for estrone that is from 341 pg/ml to 533 pg/ml; an AUC₍₀₋ₜ₎ for total estrone that is from 161 ng·h/ml to 252 ng·h/ml; and a Cₘₐₓ for total estrone that is from 28 ng/ml to 44 ng/ml. In some embodiments, the one or more pharmacokinetic parameters are measured about 18 hours, about 24 hours, about 18-36 hours, about 20-30 hours, about 22-26 hours, about 24-36 hours, about 36 hours, about 36-48 hours, about 40-48 hours, or about 48 hours after administration of the single dose.

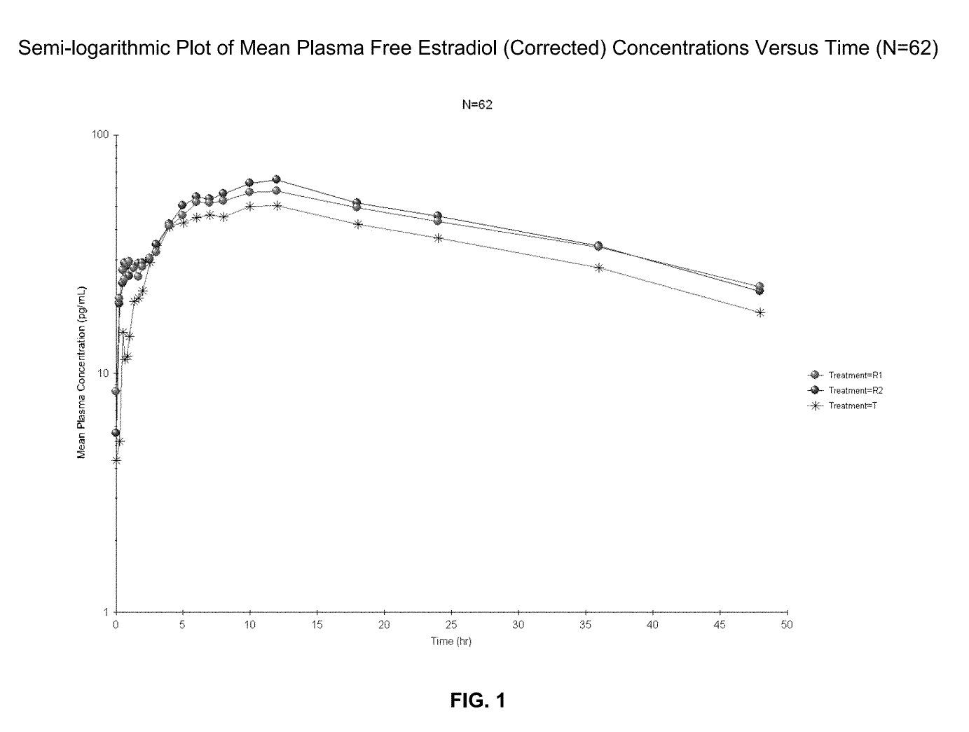

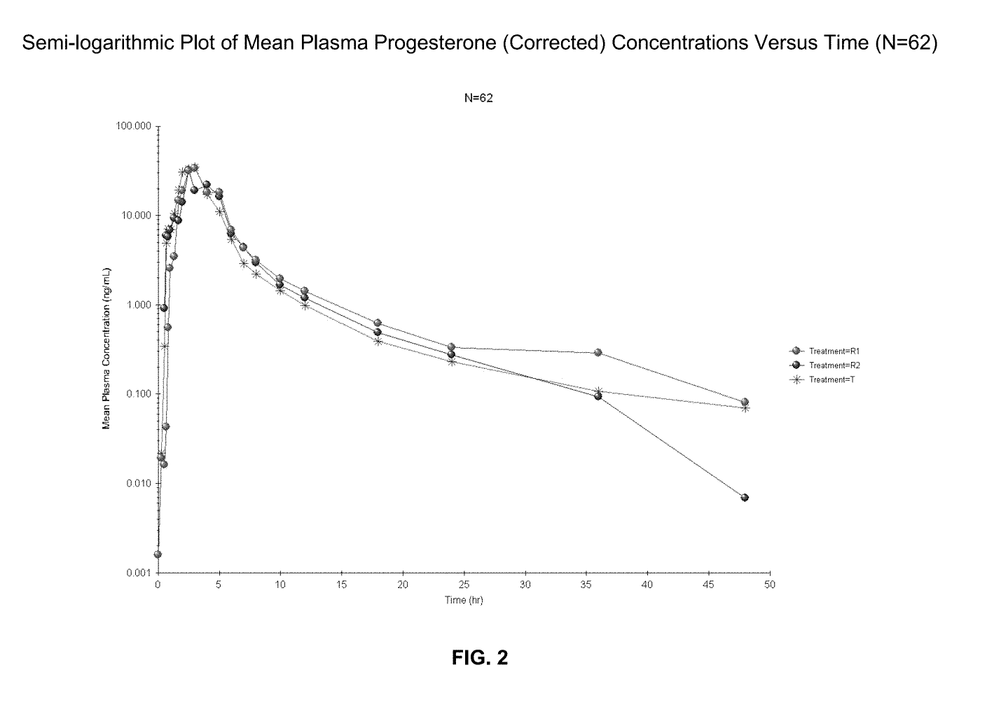

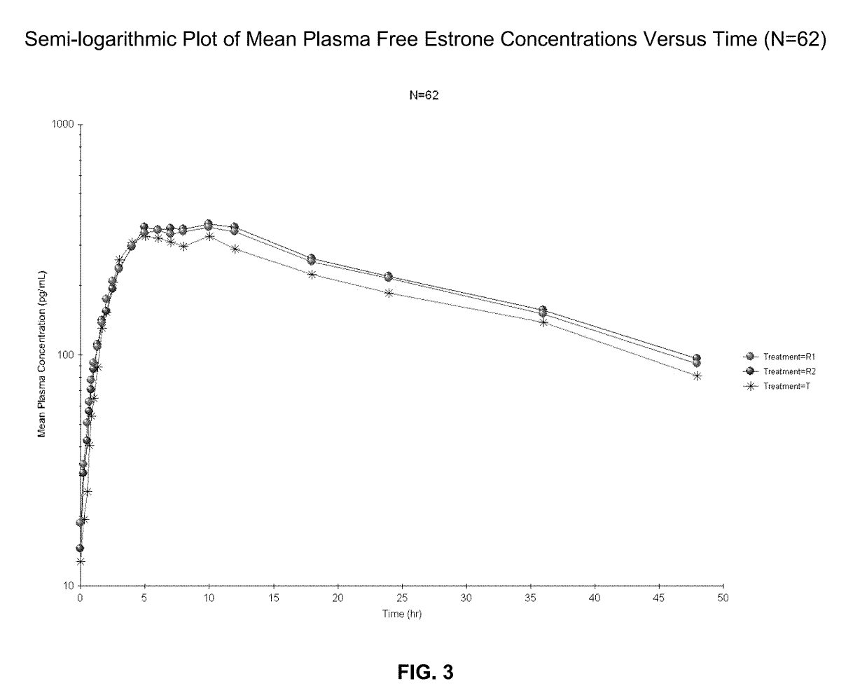

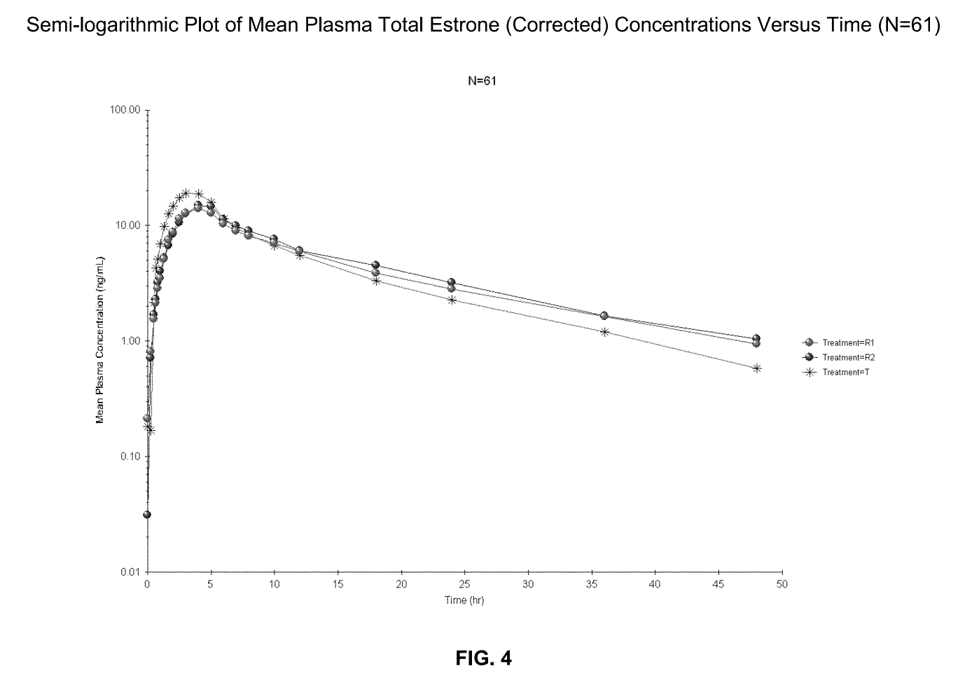

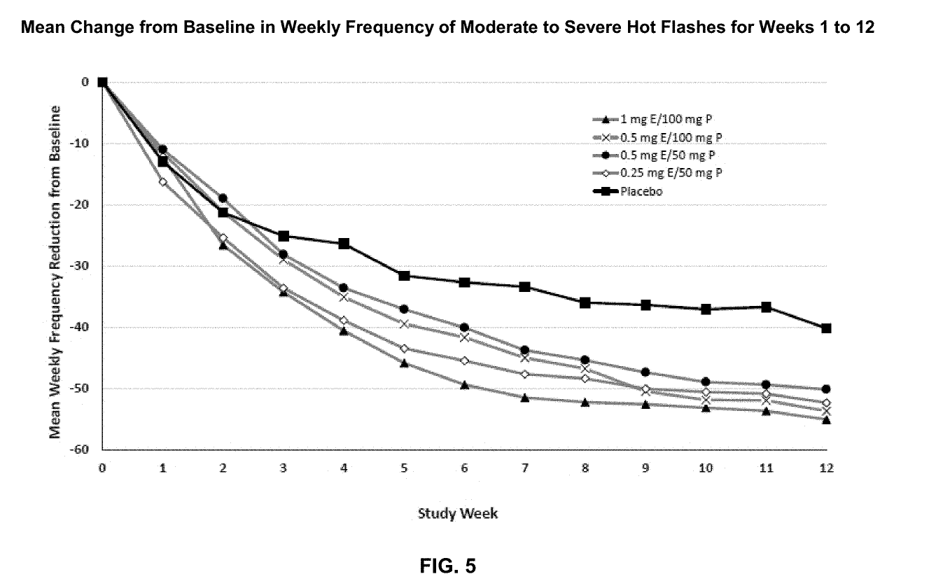

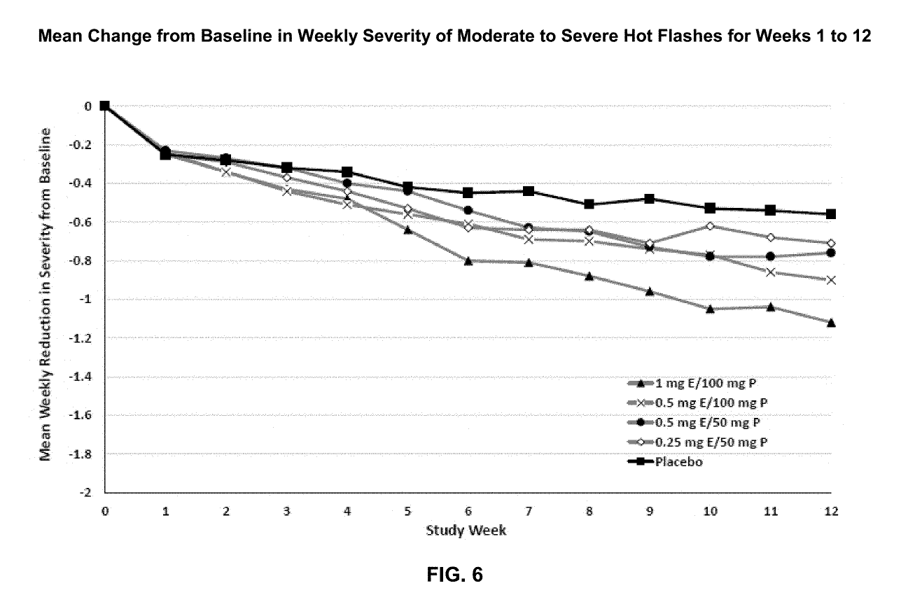

In some embodiments, administration of the pharmaceutical composition as described herein results in the blood plasma estradiol concentration profile of FIG. 1. In some embodiments, administration of the pharmaceutical composition results in the blood plasma progesterone concentration profile of FIG. 2. In some embodiments, administration of the pharmaceutical composition results in the blood plasma estrone concentration profile of FIG. 3. In some embodiments, administration of the pharmaceutical composition results in the blood plasma total estrone concentration profile of FIG. 4. In some embodiments, administration of the pharmaceutical compositions as described herein provide mean change from baseline in weekly frequency of moderate to severe hot flashes/flushes for weeks 1 to 12 as shown in FIG. 5. In some embodiments, administration of the pharmaceutical compositions as described herein provide mean change from baseline in weekly severity of moderate to severe hot flashes/flushes for weeks 1 to 12 as shown in FIG. 6.

### Administration and Treatment

Pharmaceutical compositions comprising estradiol and progesterone as described herein (e.g., compositions comprising solubilized estradiol, suspended progesterone, and a medium chain solubilizing agent) can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. Pharmaceutical compositions can be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal, or intraurethral injection or infusion. In some embodiments, administration is by injection, that is, intravenously, intramuscularly, intracutaneously, subcutaneously, intraduodenally, or intraperitoneally.

For preparing pharmaceutical compositions from the compounds of this disclosure, the pharmaceutically acceptable compositions can be either solid or liquid. Solid form preparations include powders, tablets, pills, capsules, cachets, suppositories, and dispersible granules. A solid preparation can comprise one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton Pa. (“Remington's”).

In general, the type of composition is selected based on the mode of administration. A pharmaceutical composition (e.g., for oral administration or delivery by injection) can be in the form of a liquid (e.g., an elixir, syrup, solution, emulsion or suspension). Alternatively, a pharmaceutical composition as described herein can take the form of a pill, tablet, or capsule containing the liquid oil, and thus, the composition can contain any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. The composition can also be formulated into a suppository disposed, for example, in a polyethylene glycol (PEG) solubilizing agent.

Administration of the compositions of this disclosure can be carried out via any of the accepted modes of administration. Thus, administration can be, for example, intravenous, topical, subcutaneous, transcutaneous, transdermal, intramuscular, oral, intra joint, parenteral, intra-arteriole, intradermal, intraventricular, intracranial, intraperitoneal, intralesional, intranasal, rectal, vaginal, or by inhalation. In some embodiments, a composition as described herein is administered orally. For example, a pharmaceutical composition as described herein can be administered via capsules such as soft capsules.

In some embodiments, a pharmaceutical composition as described herein is administered once daily for a period of 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 days or more. In some embodiments, a pharmaceutical composition as described herein is administered daily for at least one week, at least two weeks, at least three weeks, at least four weeks, at least one month, at least two months, at least three months, at least four months, at least five months, at least six months, at least seven months, at least eight months, at least nine months, at least ten months, at least eleven months, at least twelve months, or more. In some embodiments, a pharmaceutical composition as described herein is administered as a continuous-combined therapy regimen.

In some embodiments, a 28-day or monthly regimen of daily doses is packaged in a single kit (e.g., a blister pack) having administration days identified to improve compliance and reduce associated symptoms, among others. In some embodiments, each daily dose contains both estradiol and progesterone. In some embodiments, one or more of the daily doses contains no estradiol or no progesterone. Daily doses that comprise no estradiol or progesterone API may be referred to as placebos. A blister pack can have a plurality of scores or perforations separating the blister pack into 28 days. Each day may further comprise a single blister or a plurality of blisters. In various embodiments, each unit dose may contain micronized or partially solubilized, or fully solubilized progesterone or solubilized estradiol in amounts as set forth herein, although other dose ranges may be contemplated. In addition, kits having other configurations are also contemplated herein. For example, without limitation, kits having such blister packs may contain any number of daily doses.

In some embodiments, the pharmaceutical compositions disclosed herein are useful in treating conditions in subjects caused, at least in part, by estrogen deficiency, particularly for women with a uterus. In some embodiments, prior to treatment, the subject has a serum estradiol level of ≤10 pg/mL. For example, in embodiments, the pharmaceutical compositions disclosed herein are useful for the treatment of one or more of the following conditions: endometrial hyperplasia; secondary amenorrhea; prevention of preterm birth, when the subject has a shortened cervix; menopause-related symptoms including, for example, vasomotor symptoms; in relation to treatment of hypoestrogenism related symptoms including, for example and without limitation, hot flashes and night sweats (vasomotor symptoms), sleep disturbances, mood changes and vulvo-vaginal atrophy; and osteoporosis and other non-menopausal disease states or conditions treated with supplemental progesterone or estrogen. In some embodiments, the pharmaceutical compositions disclosed herein are useful in treating vasomotor symptoms, including but not limited to, hot flashes and night sweats. In some embodiments, the pharmaceutical compositions disclosed herein are useful in treating hot flashes and night sweats. In some embodiments, the pharmaceutical compositions disclosed herein are useful in treating hot flashes. Thus, in some embodiments, this disclosure provides methods of treating such a condition by administering to the subject a composition comprising estradiol and progesterone as described herein.

### III. EXAMPLES

The following examples are offered to illustrate, but not to limit, the claimed subject matter.

### Example 1

In an exemplary embodiment, a soft gelatin capsule contains a pharmaceutical composition comprising suspended progesterone and solubilized estradiol:

TABLE 6

| | Mass | | Qty/Capsule |
|---|---|---|---|
| Ingredient | (mg) | % w/w | (mg) |
| Progesterone, USP, micronized | 50.00 | 7.14 | 50.00 |
| Estradiol Hemihydrate, USP | 2.03 | 0.29 | 2.03 |
| CAPMUL ® MCM, NF | | 82.57 | 577.97 |
| GELUCIRE ® 44/14, NF | | 10.0 | 70.00 |
| TOTAL | | 100.00 | 700.00 |

The encapsulated pharmaceutical composition of Table 6 may be manufactured in any suitable manner. For the purposes of this Example, mixing may be facilitated by an impellor, agitator, or other suitable means. Also for the purposes of this Example, heating or mixing may be performed under an inert or relatively inert gas atmosphere, such as nitrogen gas (N₂). Mixing or heating for the purposes of this Example may be performed in any suitable vessel, such as a stainless-steel vessel.

For example, CAPMUL® MCM may be heated to between 30° C. to 50° C., more preferably from 35° C. to 45° C., and more preferably to 40° C.±2° C. GELUCIRE® 44/14 may be added to the CAPMUL® MCM and mixed until dissolved (to increase the solubility of progesterone in the final solution, GELUCIRE® 44/14 was added at about 10% w/w). The addition may occur all at once or may occur gradually over a period of time. Heat may continue to be applied during the mixing of the GELUCIRE® 44/14 and the CAPMUL® MCM.

Heat may be removed from the GELUCIRE® 44/14 and CAPMUL® MCM mixture. Estradiol Hemihydrate may be added to the mixture. The addition may occur all at once or may occur gradually over a period of time. Micronized progesterone may then be added to the GELUCIRE® 44/14, CAPMUL® MCM and Estradiol Hemihydrate mixture until dissolved. The addition may occur all at once or may occur gradually over a period of time.

### Example 2

An example of the final scale-up formulation is provided in Table 7. To manufacture, CAPMUL® MCM is heated to 40° C. GELUCIRE® 44/14 is heated to 65° C. and added and mixed until dissolved. Heat is removed. Estradiol is added and mixed until dissolved. Micronized progesterone is then added and mixed until fully suspended.

TABLE 7
Quantitative Formula: Batch Size 10,000 capsules

| | | Label | | Qty/ | Amount/ |
|---|---|---|---|---|---|
| Item | | Claim | | Capsule | Batch |
| No. | Ingredient | (mg) | % w/w | (mg) | (kg) |
| 1. | Progesterone, USP, | 50.00 | 7.14 | 50.00 | 0.50 |
| | micronized | | | | |
| 2. | Estradiol Hemihydrate, | 2.03 | 0.29 | 2.03 | 0.02 |
| | USP | | | | |
| 3. | CAPMUL ® MCM, | | 82.57 | 577.97 | 5.78 |
| | NF | | | | |
| 4. | GELUCIRE ® 44/14, | | 10.0 | 70.00 | 0.70 |
| | NF | | | | |
| | | Total: | 100.00 | 700.00 | 7.00 |

### Example 3

In an exemplary embodiment, a soft gelatin capsule contains a pharmaceutical composition having fully solubilized estradiol and partially solubilized progesterone comprising:

TABLE 8

| | | Label | | Qty/ | Amount/ |
|---|---|---|---|---|---|
| Item | | Claim | | Capsule | Batch |
| No. | Ingredient | (mg) | % w/w | (mg) | (g) |
| 1. | Progesterone, USP, | 50.00 | 25.000 | 50.00 | 500.00 |
| | micronized | | | | |
| 2. | Estradiol Hemihydrate | 0.25 | 0.129 | 0.26 | 2.58 |
| 3. | CAPMUL ® MCM, | | 73.371 | 146.74 | 1467.42 |
| | NF | | | | |
| 4. | GELUCIRE ® 44/14, | | 1.500 | 3.00 | 30.00 |
| | NF | | | | |
| | | Total: | 100.000 | 200.00 mg | 2000.00 |

To manufacture, CAPMUL® MCM is heated to 65° C. GELUCIRE® 44/14 is added and mixed until dissolved. Heat is removed. Estradiol is added and mixed until dissolved. Micronized progesterone is then added and dispersed. The mixture is then passed through a colloid mill. The resultant fill mass can be used for encapsulation.

### Example 4

In an exemplary embodiment, a soft gelatin capsule contains a pharmaceutical composition having fully solubilized estradiol and partially solubilized progesterone comprising:

TABLE 9

| | | Label | | Qty/ | Amount/ |
|---|---|---|---|---|---|
| Item | | Claim | | Capsule | Batch |
| No. | Ingredient | (mg) | % w/w | (mg) | (g) |
| 1. | Progesterone, USP, | 200.00 | 33.33 | 200.0 | 2000.0 |
| | micronized | | | | |
| 2. | Estradiol Hemihydrate | 2.00 | 0.35 | 2.07 | 20.7 |
| 3. | CAPMUL ® MCM, | | 65.32 | 391.93 | 3919.3 |
| | NF | | | | |
| 4. | GELUCIRE ® 44/14, | | 1.00 | 6.0 | 60.0 |
| | NF | | | | |
| | | Total: | 100.00 | 600.0 mg | 6000.0 |

To manufacture, CAPMUL® MCM is heated to 65° C. GELUCIRE® 44/14 is added and mixed until dissolved. Heat is removed. Estradiol is added and mixed until dissolved. Micronized progesterone is then added and dispersed. The mixture is then passed through a colloid mill. The resulting pharmaceutical composition is encapsulated in soft gelatin capsules. Alternatively, GELUCIRE® 44/14 is heated to 65° C. and CAPMUL® MCM is heated to 40° C.±5° C. to achieve mixing of the oil and the surfactant before heat is removed; estradiol is added while the mixture is cooling; progesterone is added when the mixture has dropped below about 40° C.; the mixture is then passed through a colloid mill one or more times, e.g., three times.

### Example 5

Pharmacokinetics of the First Combination 17β-Estradiol/Progesterone Capsule in Clinical Development for Hormone Therapy

The objective of this study was to evaluate the pharmacokinetic and oral bioavailability of a combination capsule of 17β-estradiol/progesterone in comparison to co-administration of the individual products ESTRACE® and PROMETRIUM®.

Subjects and Study Design:

An open label, balanced, randomized, single-dose, 2-treatment, 3-period, 3-sequence, crossover, partial-replicate, reference-scaled, oral, relative bioavailability study compared the bioavailability of an investigational 2-mg 17β-estradiol/200-mg progesterone combination capsule, without peanut oil (formulated in a manner similar to that set forth in Table 9), with that of co-administered 200-mg PROMETRIUM® (progesterone) and 2-mg ESTRACE® (17β-estradiol) tablets in healthy postmenopausal women aged 40-65 years (N=66). Key inclusion criteria for subjects included a BMI 18.50 to 29.99 kg/m² who were nonsmokers or ex-smokers (no smoking in the last 3 months). Key exclusion criteria for subjects included consuming grapefruit juice or poppy-containing foods within 48 hours before and throughout the study, use of any hormonal agent within 14 days before the study, and use of menopausal hormone therapy within 6 months before dosing.

Patients were randomly assigned sequentially to 1 of 3 dosing sequences of the same dose of the combination capsule (Test, T) and reference products (Reference, R): TRR, RTR, or RRT. 66 subjects were randomized and 62 (94.0%) completed the study. Subjects had a mean age of 49.5±5.6 years (range 40 to 64) and a mean BMI of 24.8±3.1 kg/m² (range 18.7-29.9).

After consuming a high-fat, high-calorie breakfast, each woman received a single dose of the combination (Test) capsule in 1 period of the study and single doses of the co-administered products (Reference) in each of the 2 remaining periods. Blood samples were collected within 75 minutes before dosing and post-dose at 0.25, 0.5, 0.67, 0.83, 1, 1.33, 1.67, 2, 2.5, 3, 4, 5, 6, 7, 8, 10, 12, 18, 24, 36, and 48 hours after dosing to determine progesterone, free (unconjugated) estradiol, and free and total (conjugated+free, including estrone sulfates) estrone concentrations. After collection of blood samples at each time point, the blood samples were centrifuged at 4000 RPM for 10 minutes at 4° C. to separate the plasma. The plasma from samples was separated into two aliquots. 1.5 mL from the plasma sample was transferred into aliquot I, and the remaining plasma sample was transferred into aliquot II. These aliquots were stored at −30° C. for interim storage, then at −70° C. until completion of the analysis.

Progesterone, estradiol, estrone, and total estrone in human plasma was determined using the LC-MS/MS method. The primary (Cₘₐₓ, AUC₀₋ₜ, and AUC_{0-∞}) and secondary (Tₘₐₓ, t_{1/2}, and Kₑ) PK parameters for each analyte were determined for each subject during each period by non-compartment analyses using baseline-adjusted concentrations. Statistical analyses were conducted using the SAS® statistical software.

Results:

The mean, standard deviation (SD), geometric mean, coefficient of variation (CV %), minimum, median, and maximum were calculated for Cₘₐₓ, AUC₀₋ₜ, AUC_{0-∞}, Tₘₐₓ, t_{1/2}, Kₑₗ, Kₑₗ_{_}_{lower}, Kₑₗ_{_}_{Upper}, and AUC_{% Extrap}_{_}_{obs} for progesterone, estradiol, estrone, and total estrone. The results are presented in Tables 10, 11, 12, and 13 below. For each of Tables 10-13, “Test Product (T)” refers to the progesterone+estradiol pharmaceutical composition, while “Reference product (R1)” and “Reference product (R2)” refers to co-administered PROMETRIUM® (progesterone) and ESTRACE® (estradiol). Blood plasma concentrations of progesterone, estradiol, estrone, and total estrone over time are also shown in FIGS. 1-4.

TABLE 10
Summary of Pharmacokinetic Parameters of Test Product (T) versus Reference
Product (R₁, R₂) for Progesterone

| | Untransformed Data (Mean ± SD) | | | | | |
|---|---|---|---|---|---|---|
| PK Parameter | N | Test Product (T) | N | Reference product (R1) | N | Reference product (R2) |
| Cₘₐₓ (ng/mL) | 62 | 89.2222 ± 149.7309 | 62 | 72.7228 ± 101.8885 | 62 | 69.7590 ± 87.0777 |
| AUC₀₋ₜ (ng · hr/mL) | 62 | 120.0869 ± 164.1385 | 62 | 125.9406 ± 152.3483 | 62 | 111.5867 ± 113.3200 |
| AUC_{0-∞} (ng · hr/mL) | 57 | 131.3817 ± 172.4806 | 57 | 142.1332 ± 160.4853 | 56 | 126.6006 ± 117.2665 |
| Tₘₐₓ (hr) | 62 | 3.00(0.83-10.00) | 62 | 3.00(1.00-12.00) | 62 | 4.00(0.67-18.00) |
| Kₑₗ (hr⁻¹) | 57 | 0.3064 ± 0.2427 | 57 | 0.2684 ± 0.1912 | 56 | 0.2795 ± 0.2475 |
| t_{1/2} | 57 | 4.6445 ± 4.5366 | 57 | 5.1555 ± 4.9794 | 56 | 5.0389 ± 4.5887 |
| Kₑₗ_Lower (hr⁻¹) | 57 | 7.6667 ± 4.6047 | 57 | 7.4123 ± 4.2164 | 56 | 7.9018 ± 3.9120 |
| Kₑₗ_Upper (hr⁻¹) | 57 | 16.2218 ± 11.0051 | 57 | 19.1728 ± 12.3801 | 56 | 18.1975 ± 10.0858 |
| AUC_Extra (%) | 57 | 4.3374 ± 2.5528 | 57 | 4.8416 ± 3.7526 | 56 | 5.1868 ± 4.1434 |

*Expressed in terms of median (range)

TABLE 11
Summary of Pharmacokinetic Parameters of Test Product
(T) versus Reference Product (R₁, R₂) for Estradiol

| | Untransformed Data (Mean ± SD) | | |
|---|---|---|---|
| PK Parameter | Test Product (T) | Reference product (R1) | Reference product (R2) |
| Cₘₐₓ (pg/mL) | 64.7902 ± 50.9833 | 69.1286 ± 33.0484 | 73.4236 ± 43.4077 |
| AUC₀₋ₜ (pg · hr/mL) | 1403.7333 ± 763.8136 | 1508.2206 ± 876.7390 | 1658.2502 ± 976.5556 |
| AUC_{0-∞}(pg · hr/mL) | 2459.4394 ± 4498.2737 | 2842.8805 ± 4582.6502 | 2110.9591 ± 1175.3995 |
| Tₘₐₓ (hr) | 9.00(0.50-36.00) | 10.0(0.50-35.12) | 10.00(0.25-36.60) |
| Kₑₗ (hr⁻¹) | 0.0438 ± 0.0197 | 0.0457 ± 0.0358 | 0.0464 ± 0.0338 |
| t_{1/2} (hr) | 31.9104 ± 95.9769 | 25.0908 ± 28.8346 | 20.8774 ± 12.0825 |
| Kₑₗ_{_{—}}_{Lower}(hr⁻¹) | 14.9472 ± 7.2715 | 14.9667 ± 7.0150 | 14.7953 ± 5.8774 |
| Kₑₗ_{_{—}}_{Upper} (hr⁻¹) | 45.3602 ± 6.3668 | 44.3277 ± 7.4003 | 43.8330 ± 7.6449 |
| AUC_Extra (%) | 22.8106 ± 16.6498 | 25.4773 ± 20.2911 | 24.9566 ± 16.4713 |

*Expressed in terms of median (range)

TABLE 12
Summary of Pharmacokinetic Parameters of Test Product
(T) versus Reference Product (R₁, R₂) for Free Estrone

| | Untransformed Data (Mean ± SD) | | |
|---|---|---|---|
| PK Parameter | Test Product (T) | Reference product (RI) | Reference product (R2) |
| Cₘₐₓ (pg/mL) | 426.5492 ± 179.3303 | 455.5107 ± 189.448 | 467.2302 ± 207.4373 |
| AUC₀₋ₜ (pg · hr/mL) | 9096.0907 ± 4377.2730 | 10156.0282 ± 5140.5831 | 10507.3557 ± 5183.1289 |
| AUC_{0-∞}(pg · hr/mL) | 11994.9695 ± 6678.5468 | 13445.9048 ± 8699.4068 | 14066.2362 ± 7563.2370 |
| Tₘₐₓ (hr) | 5.50(0.83-36.00) | 8.00(1.67-18.00) | 10.00(1.67-18.00) |
| Kₑₗ (hr⁻¹) | 0.0399 ± 0.0146 | 0.0424 ± 0.0172 | 0.0406 ± 0.0209 |
| t_{1/2} (hr) | 20.3172 ± 9.4052 | 19.4595 ± 9.8711 | 20.7515 ± 9.3985 |
| Kₑₗ_{_{—}}_{Lower}(hr⁻¹) | 13.8443 ± 7.0649 | 14.8871 ± 6.6459 | 14.9194 ± 6.4485 |
| Kₑₗ_{_{—}}_{Upper} (hr⁻¹) | 46.0238 ± 5.5080 | 46.2547 ± 5.3060 | 46.2244 ± 5.3126 |
| AUC_Extra (%) | 21.2980 ± 11.2283 | 20.3648 ± 11.1060 | 21.8900 ± 11.8537 |

*Expressed in terms of median (range)

TABLE 13
Summary of Pharmacokinetic Parameters of Test Product (T) versus Reference
Product (R₁, R₂) for Total Estrone

| | | Untransformed Data (Mean ± SD) | | | | |
|---|---|---|---|---|---|---|
| | | | | Reference | | Reference product |
| PK Parameter | N | Test Product (T) | N | product (R1) | N | (R2) |
| Cₘₐₓ (ng/mL) | 61 | 35.4289 ± 17.0856 | 61 | 19.8716 ± 7.4485 | 61 | 19.9048 ± 8.0288 |
| AUC₀₋ₜ (ng · hr/mL) | 61 | 201.7524 ± 94.2081 | 61 | 182.7729 ± 88.8386 | 61 | 199.8295 ± 94.9392 |
| AUC_{0-∞} (ng · hr/mL) | 61 | 213.2402 ± 104.6011 | 60 | 193.6387 ± 100.5831 | 56 | 203.0289 ± 81.4884 |
| Tₘₐₓ (hr) | 61 | 2.50(0.67−7.00) | 61 | 4.00(1.33−18.00) | 61 | 4.00(1.33−10.00) |
| Kₑₗ (hr⁻¹) | 61 | 0.0799 ± 0.0398 | 60 | 0.0803 ± 0.0399 | 56 | 0.0718 ± 0.0243 |
| t_{1/2} (hr) | 61 | 10.3619 ± 4.0023 | 60 | 9.8448 ± 3.0702 | 56 | 10.7830 ± 3.6624 |
| Kₑₗ_Lower (hr⁻¹) | 61 | 13.0492 ± 6.8585 | 60 | 13.5945 ± 8.0129 | 56 | 11.8870 ± 6.8696 |
| Kₑₗ_Upper (hr⁻¹) | 61 | 45.3979 ± 6.6589 | 60 | 46.3775 ± 5.2525 | 56 | 46.7054 ± 4.3888 |
| AUC_Extra (%) | 61 | 4.5030 ± 3.7366 | 60 | 4.5913 ± 3.4953 | 56 | 5.3450 ± 3.9831 |

*Expressed in terms of median (range)

### Example 6

Pharmacokinetic data (Cₘₐₓ, AUC₍₀₋ₜ₎, AUC_{(0-∞)}, and Tₘₐₓ) for progesterone, estradiol, free estrone, and total estrone is presented in Tables 14-17. Pharmaceutical compositions A-E are disclosed in Tables 1-5. The pK values for pharmaceutical composition E were calculated as disclosed in Example 5. For pharmaceutical compositions A-D, expected pharmacokinetic data is calculated from the data disclosed for pharmaceutical composition E.

TABLE 14
Summary of Pharmacokinetic Parameters of the Pharmaceutical Compositions
of Tables 1−5 for Progesterone

| Pharmaceutical | Progesterone | Estradiol | Cₘₐₓ | AUC₍₀₋ₜ₎ | AUC_{(0-∞)} | Tₘₐₓ |
|---|---|---|---|---|---|---|
| Composition | Content | Content | (ng/mL) | (ng · hr/mL) | (ng · hr/mL) | (hr) |
| A | 50 mg | 0.25 mg | 22.30555 | 30.0217 | 32.8454 | 3.00 |
| B | 50 mg | 0.50 mg | 22.3055 | 30.0217 | 32.8454 | 3.00 |
| C | 100 mg | 0.50 mg | 44.6111 | 60.0435 | 65.6909 | 3.00 |
| D | 100 mg | 1 mg | 44.6111 | 60.0435 | 65.6909 | 3.00 |
| E | 200 mg | 2 mg | 89.2222 | 120.0869 | 131.3817 | 3.00 |

TABLE 15
Summary of Pharmacokinetic Parameters of the Pharmaceutical Compositions
of Tables 1−5 for Estradiol

| Pharmaceutical | Progesterone | Estradiol | Cₘₐₓ | AUC₍₀₋ₜ₎ | AUC_{(0-∞)} | Tₘₐₓ |
|---|---|---|---|---|---|---|
| Composition | Content | Content | (pg/mL) | (pg · hr/mL) | (pg · hr/mL) | (hr) |
| A | 50 mg | 0.25 mg | 8.0988 | 175.4667 | 307.4299 | 9.00 |
| B | 50 mg | 0.50 mg | 16.1976 | 350.9333 | 614.8599 | 9.00 |
| C | 100 mg | 0.50 mg | 16.1976 | 350.9333 | 614.8599 | 9.00 |
| D | 100 mg | 1 mg | 32.3951 | 701.8667 | 1229.7197 | 9.00 |
| E | 200 mg | 2 mg | 64.7902 | 1403.7333 | 2459.4394 | 9.00 |

TABLE 16
Summary of Pharmacokinetic Parameters of the Pharmaceutical Compositions
of Tables 1−5 for Free Estrone

| Pharmaceutical | Progesterone | Estradiol | Cₘₐₓ | AUC₍₀₋ₜ₎ | AUC_{(0-∞)} | Tₘₐₓ |
|---|---|---|---|---|---|---|
| Composition | Content | Content | (pg/mL) | (pg · hr/mL) | (pg · hr/mL) | (hr) |
| A | 50 mg | 0.25 mg | 53.3187 | 1137.0113 | 1499.3712 | 5.50 |
| B | 50 mg | 0.50 mg | 106.6373 | 2274.0227 | 2998.7424 | 5.50 |
| C | 100 mg | 0.50 mg | 106.6373 | 2274.0227 | 2998.7424 | 5.50 |
| D | 100 mg | 1 mg | 213.2746 | 4548.0454 | 5997.4848 | 5.50 |
| E | 200 mg | 2 mg | 426.5492 | 9096.0907 | 11994.9695 | 5.50 |

TABLE 17
Summary of Pharmacokinetic Parameters of the Pharmaceutical Compositions
of Tables 1−5 for Total Estrone

| Pharmaceutical | Progesterone | Estradiol | Cₘₐₓ | AUC₍₀₋ₜ₎ | AUC_{(0-∞)} | Tₘₐₓ |
|---|---|---|---|---|---|---|
| Composition | Content | Content | (ng/mL) | (ng · hr/mL) | (ng · hr/mL) | (hr) |
| A | 50 mg | 0.25 mg | 4.4286 | 25.2191 | 26.6550 | 2.50 |
| B | 50 mg | 0.50 mg | 8.8572 | 50.4381 | 53.3101 | 2.50 |
| C | 100 mg | 0.50 mg | 8.8572 | 50.4381 | 53.3101 | 2.50 |
| D | 100 mg | 1 mg | 17.7145 | 100.8762 | 106.6201 | 2.50 |
| E | 200 mg | 2 mg | 35.4289 | 201.7524 | 213.2402 | 2.50 |

The ranges of expected pK values for each of the pharmaceutical compositions of Tables 1-4 are disclosed in Tables 18-21, respectively.

TABLE 18
pK Ranges for the Pharmaceutical Composition
of Table 1 (Pharmaceutical Composition A)

| | Cₘₐₓ | AUC₍₀₋ₜ₎ | AUC_{(0-∞)} |
|---|---|---|---|
| Progesterone | 17.8444 ng/mL to | 24.0174 ng · hr/mL to | 26.2763 ng · hr/mL to |
| | 27.8819 ng/mL | 37.5272 ng · hr/mL | 41.0568 ng · hr/mL |
| Estradiol | 6.4790 pg/mL to | 140.3733 pg · hr/mL to | 245.9439 pg · hr/mL to |
| | 10.1235 pg/mL | 219.3333 pg · hr/mL | 384.2874 pg · hr/mL |
| Free estrone | 42.6549 pg/mL to | 909.6091 pg · hr/mL to | 1199.4970 pg · hr/mL to |
| | 66.6483 pg/mL | 1421.2642 pg · hr/mL | 1874.2140 pg · hr/mL |
| Total estrone | 3.5429 ng/mL to | 20.1752 ng · hr/mL to | 21.3240 ng · hr/mL to |
| | 5.5358 ng/mL | 31.5238 ng · hr/mL | 33.3188 ng · hr/mL |

TABLE 19
pK Ranges for the Pharmaceutical Composition
of Table 2 (Pharmaceutical Composition B)

| | Cₘₐₓ | AUC₍₀₋ₜ₎ | AUC_{(0-∞)} |
|---|---|---|---|
| Progesterone | 17.8444 ng/mL to | 24.0174 ng · hr/mL to | 26.2763 ng · hr/mL to |
| | 27.8819 ng/mL | 37.5272 ng · hr/mL | 41.0568 ng · hr/mL |
| Estradiol | 12.9580 pg/mL to | 280.7467 pg · hr/mL to | 491.8879 pg · hr/mL to |
| | 20.2469 pg/mL | 438.6667 pg · hr/mL | 768.5748 pg · hr/mL |
| Free estrone | 85.3098 pg/mL to | 1819.2181 pg · hr/mL to | 2398.9939 pg · hr/mL to |
| | 133.2966 pg/mL | 2842.5283 pg · hr/mL | 3748.4280 pg · hr/mL |
| Total estrone | 7.0858 ng/mL to | 40.3505 ng · hr/mL to | 42.6480 ng · hr/mL to |
| | 11.0715 ng/mL | 63.0476 ng · hr/mL | 66.6376 ng · hr/mL |

TABLE 20
pK Ranges for the Pharmaceutical Composition
of Table 3 (Pharmaceutical Composition C)

| | Cₘₐₓ | AUC₍₀₋ₜ₎ | AUC_{(0-∞)} |
|---|---|---|---|
| Progesterone | 35.6889 ng/mL to | 48.0348 ng · hr/mL to | 52.5527 ng · hr/mL to |
| | 55.7639 ng/mL | 75.0543 ng · hr/mL | 82.1136 ng · hr/mL |
| Estradiol | 12.9580 pg/mL to | 280.7467 pg · hr/mL to | 491.8879 pg · hr/mL to |
| | 20.2469 pg/mL | 438.6667 pg · hr/mL | 768.5748 pg · hr/mL |
| Free estrone | 85.3098 pg/mL to | 1819.2181 pg · hr/mL to | 2398.9939 pg · hr/mL to |
| | 133.2966 pg/mL | 2842.5283 pg · hr/mL | 3748.4280 pg · hr/mL |
| Total estrone | 7.0858 ng/mL to | 40.3505 ng · hr/mL to | 42.6480 ng · hr/mL to |
| | 11.0715 ng/mL | 63.0476 ng · hr/mL | 66.6376 ng · hr/mL |

TABLE 21
pK Ranges for the Pharmaceutical Composition
of Table 4 (Pharmaceutical Composition D)

| | Cₘₐₓ | AUC₍₀₋ₜ₎ | AUC_{(0-∞)} |
|---|---|---|---|
| Progesterone | 35.6889 ng/mL to | 48.0348 ng · hr/mL to | 52.5527 ng · hr/mL to |
| | 55.7639 ng/mL | 75.0543 ng · hr/mL | 82.1136 ng · hr/mL |
| Estradiol | 25.9161 pg/mL to | 561.4933 pg · hr/mL to | 983.7758 pg · hr/mL to |
| | 40.4939 pg/mL | 877.3333 pg · hr/mL | 1537.1496 pg · hr/mL |
| Free estrone | 170.6197 pg/mL to | 3638.4363 pg · hr/mL to | 4797.9878 pg · hr/mL to |
| | 266.5933 pg/mL | 5685.0567 pg · hr/mL | 7496.8559 pg · hr/mL |
| Total estrone | 14.1716 ng/mL to | 80.7010 ng · hr/mL to | 85.2961 ng · hr/mL to |
| | 22.1431 ng/mL | 126.0953 ng · hr/mL | 133.2751 ng · hr/mL |

### Example 7

A Phase 3, Double-Blind, Placebo-Controlled, Randomized, Multi-Center Study to Evaluate the Safety and Efficacy of Estradiol in Combination with Progesterone in Postmenopausal Women with an Intact Uterus

This study had the following two main objectives: (1) Vasomotor symptoms (VMS): To determine whether the TX-001HR given in a continuous fashion is effective at reducing the frequency and severity of moderate to severe vasomotor symptoms associated with menopause when compared with placebo treatment at weeks 4 and 12. (2) Endometrial hyperplasia: To determine whether TX-001HR given in a continuous fashion is effective at achieving a ≤1% incidence rate of endometrial hyperplasia following 12 months of therapy.

A. Study Design/Description

1. Study Design Overview

This was a prospective, randomized, double-blind, placebo-controlled, parallel group, multicenter trial to determine whether estradiol/progesterone combinations (TX-001HR) given in a continuous fashion are effective at reducing the frequency and severity of vasomotor symptoms associated with menopause as well as establishing an appropriate progesterone dose by demonstrating an acceptably low incidence of endometrial hyperplasia.

Postmenopausal subjects with an intact uterus who met the study entry criteria were randomized to one of five treatment arms (four active and one placebo) and received blinded study medication for 12 months. During the Screening period, all subjects were provided with a diary to self-assess the frequency and severity of their vasomotor symptoms. Subjects experiencing a minimum daily frequency of ≥7 (or ≥50 per week) moderate to severe hot flushes participated in a VMS Substudy during the first 12 weeks of treatment. The Substudy subjects were stratified by treatment arm within the sites, and only Substudy subjects had the possibility of being randomized to placebo. Subjects who qualified for the study except for experiencing a minimum daily frequency of ≥7 (or ≥50 per week) moderate to severe hot flushes were stratified by treatment arm within sites to one of the four active treatment arms and received blinded study medication for 12 months, but did not participate in the VMS Substudy. (However, VMS information was collected from all subjects during the first 12 weeks of treatment.

Postmenopausal women with an intact uterus who sought relief from hot flushes and met all other inclusion/exclusion criteria were eligible for 12 months of study treatment. A subset of All Study Subjects who had ≥7 per day or ≥50 moderate to severe hot flushes per week (as determined during Screening) were eligible for the 12-week VMS Substudy and for a total of 12 months of study treatment.

Clinical evaluations were performed at the following time points:
Screening Period/Baseline (—approximately 60 Days)
Visit 1 Randomization (Week 0) (Day 1)
Visit 2 Interim (Week 4, Day 28 [±3 d])
Visit 3 Interim (Week 8, Day 56 [±3 d])
Visit 4 Interim (Week 12, Day 84 [±3 d])
Visit 5 Interim (Month 6, Day 180 [±4 d])
Visit 6 Interim (Month 9, Day 270 [±4 d])
Visit 7 End of Treatment (Month 12, Day 360 [±4 d])
Post Treatment Phone Contact Safety follow up visit (approximately 15 days after the last dose of study medication)

Unscheduled visits were allowed by investigator discretion for safety reasons, administrative reasons (e.g., re-supply of study medication or diaries), or to address subject concerns or questions about the study.

2. Study Sequence and Duration of Subject Participation

The study involved a Screening period of approximately 60 days before randomization, approximately 12 months (13 cycles) of treatment, and a 15 day follow up period for a total duration of up to approximately 14.5 months of participation. See, Table 22 below:

B. Study Population

Approximately 120 investigative sites in the United States recruited subjects for participation in this study. A sufficient number of subjects were screened in order for approximately 1750 subjects to be randomized (400/active treatment group; 150 placebo group). For the VMS Substudy, approximately 750 subjects were randomized (150 per treatment group). Enrollment among treatment centers was competitive, and discontinued subjects were not replaced.

1. Inclusion Criteria

To participate in the study, a subject MUST: (1) be a female between the ages of 40 and 65 years (at the time of randomization) who is willing to participate in the study, as documented by signing the informed consent form; (2) be a postmenopausal woman with an intact uterus and a Screening serum estradiol level of ≤50 pg/mL. Postmenopausal is defined herein as: (a) ≥12 months of spontaneous amenorrhea, or (b) at least 6 months of spontaneous amenorrhea with a Screening serum FSH level of >40 mIU/ml, or (c) ≥6 weeks postsurgical bilateral oophorectomy; (3) be seeking treatment or relief for vasomotor symptoms associated with menopause; (4) to participate in the VMS Substudy, a subject must also report ≥7 moderate to severe hot flushes per day, or ≥50 per week, at the baseline assessment during Screening; subjects whose hot flushes are less frequent may still participate as non-Substudy subjects. [Note: A minimum of 14 consecutive days of complete hot flush diary data are required during the baseline assessment at Screening, and these consecutive days must occur within the last 14 days prior to the Randomization visit (not counting the Randomization visit day itself). The most recent 7 consecutive days of data prior to randomization (Day −7 to Day −1) will be used to determine the baseline number of mild, moderate and severe hot flushes for each subject.]; (5) have a Body Mass Index (BMI) less than or equal to 34 kg/m′ (BMI values should be rounded to the nearest integer [e.g., 34.4 rounds down to 34, while 26.5 rounds up to 27]); (6) be willing to abstain from using products (other than study medication) that contain estrogen, progestin, or progesterone throughout study participation; (7) be judged by the principal or sub-investigator physician as being in otherwise generally good health based on a medical evaluation performed during the Screening period prior to the initial dose of study medication. The medical evaluation findings must include: (a) a normal or non-clinically significant physical examination, including vital signs (sitting blood pressure, heart rate, respiratory rate and temperature). Sitting systolic blood pressure must be ≤140 mmHg and diastolic blood pressure must be ≤90 mmHg at Screening. A subject may be taking up to two antihypertensive medications; (b) a normal or non-clinically significant pelvic examination; (c) a mammogram that shows no sign of significant disease (can be performed within previous 6 months prior to initial dose of study medication). Subjects must have a BI-RADS 1 or 2 to enroll in the study. An incomplete mammogram result, i.e. BI-RADS 0, is not acceptable. The site must obtain a copy of the official report for the subject's study file, and it must be verified that the mammogram itself is available if needed for additional assessment; (d) a normal or non-clinically significant clinical breast examination. An acceptable breast examination is defined as no masses or other findings identified that are suspicious of malignancy; (e) a normal Screening Papanicolaou (“Pap”) smear. (Subjects with findings of atypical glandular cells [AGC], AGUS, ASCUS with high risk HPV type upon reflex testing, LSIL, ASC-H, HSIL, dysplastic cells, or malignant cells must be excluded from randomization); (f) an acceptable result from an evaluable Screening endometrial biopsy. The endometrial biopsy reports by the two central pathologists at Screening must each specify one of the following: proliferative endometrium; weakly proliferative endometrium; disordered proliferative pattern; secretory endometrium; endometrial tissue other (including benign, inactive or atrophic fragments of endometrial epithelium, glands, stroma, etc.); endometrial tissue insufficient for diagnosis; no endometrium identified; or no tissue identified. However, at least one pathologist must identify sufficient tissue to evaluate the biopsy. Additionally, the endometrial biopsy reports by the two central pathologists of Other Findings at Screening must each specify one of the following: endometrial polyp not present; benign endometrial polyp; or polyp other. See Exclusion criteria #27 and #28; (g) a normal or non-clinically significant 12-lead ECG.

2. Exclusion Criteria

To participate in the study, a subject must NOT: (1) be currently hospitalized; (2) have a history of thrombosis of deep veins or arteries or a thromboembolic disorder; (3) have a history of coronary artery or cerebrovascular disease (e.g., myocardial infarction, angina, stroke, TIA); (4) have a history of a chronic liver or kidney dysfunction/disorder (e.g., Hepatitis C or chronic renal failure); (5) have a history of a malabsorption disorder (e.g., gastric bypass, Crohn's disease); (6) have a history of gallbladder dysfunction/disorders (e.g., cholangitis, cholecystitis), unless gallbladder has been removed; (7) have a history of diabetes, thyroid disease or any other endocrinological disease. (Subjects with diet-controlled diabetes or controlled hypothyroid disease at Screening are not excluded); (8) have a history of estrogen-dependent neoplasia; (9) have a history of atypical ductal hyperplasia of the breast; (10) have a finding of clinically significant uterine fibroids at Screening; (11) have had a uterine ablation; (12) have a history of undiagnosed vaginal bleeding; (13) have any history of endometrial hyperplasia, melanoma, or uterine/endometrial, breast or ovarian cancer; (14) have any history of other malignancy within the last 5 years, with the exception of basal cell (excluded if within 1 year) or non-invasive squamous cell (excluded if within 1 year) carcinoma of the skin; (15) have a history of any other cardiovascular, hepatic, renal, pulmonary, hematologic, gastrointestinal, endocrine, immunologic, dermatologic, neurologic, psychological (e.g., bipolar disorder, schizophrenia, major depressive disorder), or musculoskeletal disease or disorder that is clinically significant in the opinion of the Principal Investigator or Medical Sub-Investigator; (16) have any of the following clinical laboratory values at Screening: (a) fasting triglyceride of ≥300 mg/dL and/or total cholesterol of ≥300 mg/dL; (b) positive laboratory finding for Factor V Leiden mutation; (c) AST or ALT ≥1.5 times the upper limit of normal (ULN); (d) fasting glucose >125 mg/dL; (17) be known to be pregnant or have a positive urine pregnancy test. (Note: A pregnancy test is not required for subjects who have had bilateral tubal ligation, bilateral oophorectomy, or are 55 years old or greater and have experienced cessation of menses for at least 1 year); (18) have contraindication to estrogen and/or progestin therapy or allergy to the use of estradiol and/or progesterone or any components of the investigational drugs; (19) use 15 or more cigarettes per day or currently use any electronic cigarettes; (20) have a history of drug and/or alcohol abuse within one year of start of study; (21) have used, within 28 days prior to the initial dose of study medication at Visit 1, any medication known to induce or inhibit CYP3A4 enzyme activity that may affect estrogen and/or progestin drug metabolism; (22) have used, within 28 days prior to Screening, or plan to use during the study, any prescription or over-the-counter (OTC) medication (including herbal products, such as St. John's Wort) that would be expected to alter progesterone or estrogen activity or is being used to treat vasomotor symptoms; (23) have used estrogen alone or estrogen/progestin, SERM (selective estrogen receptor modulator), testosterone, or estrogen/testosterone for any of the following time periods: (a) vaginal nonsystemic hormonal products (rings, creams, gels) within 7 days prior to Screening, or vaginal systemic products (e.g., FemRing) within 28 days prior to Screening; (b) transdermal estrogen alone or estrogen/progestin products within 8 weeks prior to Screening; (c) Oral estrogen and/or progestin therapy and/or SERM within 8 weeks prior to Screening; (d) Progestational implants, estrogen or estrogen/progestational injectable drug therapy within 3 months prior to Screening; (e) estrogen pellet therapy or progestational injectable drug therapy within 6 months prior to Screening; (f) percutaneous estrogen lotions/gels within 8 weeks prior to Screening; (g) oral, topical, vaginal, patch, implantable or injectable androgen therapy within 8 weeks prior to Screening; (24) have used an intrauterine device (IUD) within the 12 weeks prior to Screening; (25) For subjects in the VMS Substudy only: use of medication that may affect the outcome of the vasomotor symptom endpoints within 28 days prior to Screening (e.g. SSRIs [selective serotonin reuptake inhibitors], SNRIs [serotonin and norepinephrine reuptake inhibitors], aldomet, dopaminergic or antidopaminergic drugs, gabapentin, clonidine, or bellergal); (26) have any reason which, in the opinion of the Principal Investigator or Medical Sub-Investigator, would prevent the subject from safely participating in the study or complying with protocol requirements; (27) have a Screening endometrial biopsy sample that is found by both primary pathologists to have endometrial tissue insufficient for diagnosis, no endometrium identified, or no tissue identified. (With the approval of the Medical Monitor, the Screening endometrial biopsy may be repeated once); (28) endometrial polyps with atypical nuclei reported by at least 1 central pathologist; (29) have contraindication to any planned study assessments (e.g., endometrial biopsy); (30) have participated in another clinical trial within 30 days prior to Screening, have received an investigational drug within the three months prior to the initial dose of study medication, or be likely to participate in a clinical trial or receive another investigational medication during the study; (31) currently use marijuana.

3. Concomitant and Prohibited Medications

Study participants were instructed not to take estrogen, SERMs, progestin or progesterone other than study medication in the specified timeframes prior to Screening outlined in Section B.2 (Exclusion Criteria) nor during the study.

The use of any medication known to induce or inhibit CYP3A4 enzyme activity that may affect estrogen/progestin drug metabolism was prohibited within 28 days prior to Visit 1 and throughout the study.

The use of any medication, herbal products or nutritional supplements known or suspected to interact with hormone therapy was prohibited within 28 days prior to Screening and throughout the study. Testosterone will be prohibited within 8 weeks prior to Screening and during the study.

For subjects in the VMS Substudy only: use of medication that may affect the outcome of the vasomotor symptom endpoints within 28 days prior to Screening and during participation in the VMS Substudy (through the first 12 weeks of treatment) was prohibited (e.g., SSRIs, SNRIs, aldomet, dopaminergic or antidopaminergic drugs, gabapentin, clonidine, or bellergal).

Subjects were instructed to report all concomitant medications, including over the counter (OTC) products and herbal or nutritional supplements/medications. Subject were also instructed to report any changes in concomitant medications; they are to be questioned by site personnel regarding concomitant medications at each site visit and, when appropriate, during contacts between visits.

C. Study Procedures and Evaluations

1. Informed Consent

Postmenopausal women 40 to 65 years of age signed a written informed consent that was approved by an Institutional Review Board (IRB). No study-related procedures or activities were performed until each subject was fully informed about the study and the consent form was properly signed and dated. The Investigator, or a qualified person designated by the Investigator, explained the purpose and procedures of the study as well as potential benefits and risks. All subjects were given a copy of the signed and dated consent form.

2. Demographics, Medical/Gynecological History, and Concomitant Medications

At Screening, a complete medical history, including demographic data (age, sex, race and ethnicity), surgical and gynecological history (date of last menstrual period, date of bilateral oophorectomy, if applicable), and use of tobacco and alcohol history was recorded. The medical history included a review of all past and current diseases. It also included the history of hot flushes. Any hormonal therapy taken within 6 months prior to this visit was recorded (using generic names, if known) with the corresponding indication. The other medications to be recorded included prescription and OTC medications, dietary supplements, and all products taken within 30 days prior to the Screening visit.

3. Physical Examination

A complete physical examination was conducted at Screening and Visit 7/End of Treatment. The physical examination included, at a minimum, examination of the subject's general appearance, HEENT (head, eyes, ears, nose and throat), heart, lungs, musculoskeletal system, gastrointestinal (GI) system, neurological system, lymph nodes, abdomen and extremities. The subject's height was measured at Screening only and body weight (while the subject is lightly clothed) was measured at Screening, Week 12, Month 6, and the End of Treatment. BMI was calculated.

A urine pregnancy test was performed at the very start of Screening. If the pregnancy test were positive, the subject was excluded from study participation. A pregnancy test was not required for subjects who had a bilateral tubal ligation, bilateral oophorectomy, or who are 55 years old or greater and have experienced cessation of menses for at least 1 year.

Vital signs (body temperature, heart rate [HR], respiration rate [RR], and sitting blood pressure [BP]) were measured after the subject has been sitting for >10 minutes.

Each subject was required to have normal or non-clinically significant pelvic and breast examinations performed prior to initial dose of study medication. The pelvic and breast exam was repeated at Visits 5 and 7/End of Treatment.

Each subject was required to have the following examinations at Screening:
Screening Pap smear for subjects with an intact cervix. (All subjects must have had a Pap smear done during Screening, regardless of any recent prior assessment.)
Mammogram (may have been performed within previous 6 months of first dose of study medication; the site must obtain a copy of the official report for the subject's study file, and it must be verified that the mammogram itself is available if needed for additional assessment. If the subject had not had a mammogram within the previous 6 months prior to the first dose of study medication or relevant documentation cannot be obtained, one was performed before the subject can be randomized

A Pap smear and mammogram were repeated at Visit 7/End of Treatment.

4. Laboratory Measurements

Blood samples for blood chemistry, hematology, coagulation tests, and hormone levels were collected. The schedule associated with laboratory measurements can be found in Table 23, Laboratory Tests Schedule, below.

TABLE 23
Laboratory Tests Schedule

| | | VISIT | VISIT | VISIT | VISIT | VISIT | VISIT | VISIT |
|---|---|---|---|---|---|---|---|---|
| Laboratory Tests^{1,2} | Screening | 1 | 2 | 3 | 4 | 5 | 6 | 7 |
| Hematology | | | | | | | | |
| Hemoglobin | X | — | — | — | X | X | X | X |
| Hematocrit | X | — | — | — | X | X | X | X |
| Red Blood Cell Count | X | — | — | — | X | X | X | X |
| White Blood Cell Count | X | — | — | — | X | X | X | X |
| with differential | | | | | | | | |
| Platelet Count | X | — | — | — | X | X | X | X |
| Serum Chemistry | | | | | | | | |
| Sodium | X | — | — | — | X | X | X | X |
| Potassium | X | — | — | — | X | X | X | X |
| Chloride | X | — | — | — | X | X | X | X |
| Bicarbonate | X | — | — | — | X | X | X | X |
| Blood urea nitrogen | X | — | — | — | X | X | X | X |
| Iron | X | — | — | — | X | X | X | X |
| Albumin | X | — | — | — | X | X | X | X |
| Total Protein | X | — | — | — | X | X | X | X |
| Aspartate | X | — | — | — | X | X | X | X |
| Aminotransferase | | | | | | | | |
| Alanine Aminotransferase | X | — | — | — | X | X | X | X |
| Alkaline Phosphatase | X | — | — | — | X | X | X | X |
| Amylase | X | — | — | — | X | X | X | X |
| Creatinine | X | — | — | — | X | X | X | X |
| Calcium | X | — | — | — | X | X | X | X |
| Phosphate | X | — | — | — | X | X | X | X |
| Uric Acid | X | — | — | — | X | X | X | X |
| Total Bilirubin | X | — | — | — | X | X | X | X |
| Glucose³ | X | — | — | — | X | X | X | X |
| Coagulogram | | | | | | | | |
| Prothrombin time, | X | — | — | — | X | X | X | X |
| Activated partial | | | | | | | | |
| thromboplastin time, | | | | | | | | |
| Fibrinogen, Antithrombin | | | | | | | | |
| III, Protein C and Protein S | | | | | | | | |
| Factor V Leiden | X | — | — | — | — | — | — | — |
| Hormone Levels | | | | | | | | |
| Follicle-stimulating | X | — | — | — | — | — | — | — |
| hormone (FSH)⁴ | | | | | | | | |
| Thyroid-stimulating | X | — | — | — | — | — | — | |
| hormone | | | | | | | | — |
| Estradiol, estrone | X | — | X | — | X | X | X | X |
| Progesterone | X | | | | X | | | X |
| Lipid Panel | | | | | | | | |
| Fasting LDL, HDL, | X | — | — | — | X | X | X | X |
| Triglycerides, Total | | | | | | | | |
| Cholesterol | | | | | | | | |
| Urinalysis | | | | | | | | |
| Appearance, pH, Specific | X | — | — | — | X | X | X | X |
| Gmvity, Protein | | | | | | | | |
| Other tests | | | | | | | | |
| Urine pregnancy test⁵ | X | — | — | — | — | — | — | — |

¹Additional tests may be performed, if necessary, based on:
Standard lab panels utilized by the clinical site;
Country regulatory requirements
Based on clinical judgment and necessity
²Normal laboratory values will be provided in study-specific laboratory manual
³Must be fasting
⁴Subjects with ≥12 months of spontaneous amenorrhea or bilateral oophorectomy excluded from FSH test
⁵A pregnancy test is not required for subjects who have had a hysterectomy, bilateral oophorectomy, or bilateral tubal ligation or are ≥55 years old and has have experienced cessation of menses for at least 1 year

Clinical laboratory tests could be repeated with prior approval of the Sponsor or designee only. The Principal Investigator or a qualified and delegated Medical Sub-Investigator were responsible for interpreting the laboratory findings (i.e., determining the clinical significance of any abnormal values indicated) and for signing and dating the laboratory report. Any clinically relevant changes requiring treatment, interruption or discontinuation of study medication occurring during the trial were reported as an adverse event.

Any authorized and qualified person was allowed to collect biological samples from the subject. The date and time of sample collection was recorded. A central laboratory was designated for this study to perform the analyses of blood and urine samples and to provide applicable kits, supplies, and instructions for the collection and handling of samples. Estradiol evaluations for screening inclusion criteria were conducted by a validated rapid chromatographic assay. All baseline and post-treatment estradiol, estrone, and progesterone were done by a validated bioanalytical assay. Sample collection and handling procedures for laboratory assessments were performed according to the procedures designated by the central laboratory. Contact information and relevant documentation regarding the central laboratory were provided separately.

5. Evaluation of Vasomotor Symptoms and Bleeding/Spotting Episodes

Screening Period:

Upon completion of the initial screening procedures, all subjects who were determined to be eligible to continue screening were provided with a Hot Flush diary that was completed for the remainder of the Screening period. Subjects were instructed to complete the diary on a daily basis by recording the number and severity of vasomotor symptoms (hot flushes) in their diaries. The severity of vasomotor symptoms is defined clinically in Table 24, below.

TABLE 24
Severity of Hot Flushes

| Severity | Description |
|---|---|
| Mild | Sensation of heat without sweating |
| Moderate | Sensation of heat with sweating, able to continue activity |
| Severe | Sensation of heat with sweating, causing cessation of activity |

A minimum of 14 consecutive days of complete hot flush diary data was required during the baseline assessment at Screening, and these consecutive days had to occur within the last 14 days prior to the Randomization visit (however, not counting the Randomization visit day itself). The most recent 7 consecutive days of data prior to randomization was used to determine the baseline number of mild, moderate and severe hot flushes for each subject.

At Visit 1, subjects who continued to meet the eligibility criteria with a minimum daily frequency of ≥7 (or ≥50 per week) moderate to severe hot flushes in the 7 days prior to Visit 1 were randomized into the VMS Substudy. All other eligible subjects not meeting the VMS Substudy hot flush requirements were randomized into the non-Substudy portion of the trial until enrollment was completed.

Treatment Period—Day 1-84

All subjects (both VMS Substudy and non-Substudy) will complete a Hot Flush/Bleeding and Spotting diary through Week 12. In addition to collecting the hot flush frequency and severity, the subject diary will also collect the date and time of study medication administration, the time of food intake closest to dosing and the intensity of bleeding/spotting episodes. Bleeding/Spotting definitions are as follows: “None”=no bleeding or spotting; “Spotting”=not requiring sanitary protection; “Bleeding”=requiring sanitary protection.

Subjects were instructed to return their diary at each study visit. Study staff reviewed the returned subject diaries to ensure proper completion; subjects were re-instructed as needed on correctly completing the diary.

Any changes made to the subject's diary entries were documented in accordance with Good Clinical Practice standards. Any change or correction to source documents were dated, initialed, explained (if necessary) and did not obscure the original entry. As appropriate, corrections were preferably documented during review of the diary while the subject was present at the site clinic. The Investigator or his/her designee were able to make self-evident corrections to subject initials, subject number, and dates when proper entries could be determined unambiguously. Corrections could be made to the diaries when appropriate to document the subject's intended meaning, but missing diary data was not to be completed by recall more than one week after the date of the event. Corrections for study drug administration could be made based on study drug reconciliation when appropriate. The study staff entered the subject diary data into the CRF promptly in order for the Sponsor to assess subject compliance with completion of the subject diaries. Subjects who did not complete the diary correctly or did not submit the diary despite repeated instructions could be discontinued for non-compliance after discussion with the Medical Monitor. If a subject forgot to return the diary to the site, the study staff made every reasonable attempt to collect the diary or any missing diary pages.

Treatment Period—Day 85-360

Upon completion of the initial 12 weeks of the Treatment Period, all subjects were requested to continue their participation and complete 12 months of therapy. At the Week 12 visit, subjects were dispensed 3 months supply of a Bleeding and Spotting diary for completion daily. Subjects were instructed to return the diary at the next scheduled visit. Subjects continued to complete the Bleeding and Spotting diary and returned the diary at each clinic visit until the End of Treatment at Month 12. The study staff continued to monitor compliance with diary completion.

6. Menopause-Specific Quality of Life Questionnaire (MENQOL), Medical Outcomes Study-Sleep Questionnaire (MOS-Sleep), and Clinical Global Impression (CGI)

The 1996 version of the Menopause-specific quality of life questionnaire (MENQOL), which was developed by Hilditch, et al., Maturitas, 1996; 24(3):161-175), was utilized to assess changes in quality of life of study participants. The MENQOL was conducted at Visits 1, 4, 5 and 7.

The 1992 version of the Medical Outcomes Study-Sleep Questionnaire (MOS-Sleep), which is described by Hays, R D and Stewart, A L, Sleep Measures, in A. L. Stewart and J. E. Ware (eds.), Measuring functioning and well-being: The Medical Outcomes Study approach (pp. 235-259), Durham, N.C.: Duke University Press, 1992), was utilized to assess changes in sleep. The MOS-Sleep questionnaire was conducted at Visits 1, 4, 5 and 7.

At weeks 4, 8 and 12, subjects in the VMS Substudy were asked to provide a Clinical Global Impression (CGI) as described by Gerlinger et al., Menopause: The Journal of The North American Menopause Society, 2012:19(7):799-803. Subjects were instructed to answer the following question: “Rate the total improvement, whether or not in your judgment it is due entirely to drug treatment. Compared to your condition at admission to the study, how much has it changed?” The subjects were asked to answer this question using a symmetric seven-point scale, as follows:
Very much improved
Much improved
Minimally improved
No change
Minimally worse
Much worse
Very much worse

7. Screening Procedures

The Screening Period began on the date that the subject signed the informed consent form. The prospective subjects visited the study center and were assessed by qualified and properly delegated study staff to verify eligibility and exclude any co-morbid conditions.

The screening evaluation period was completed within 60 days; however, the period may have been longer with the approval of the Medical Monitor. All Screening assessments were completed prior to randomization. Completion of Screening procedures typically required at least two clinic visits prior to randomization at Visit 1.

Subjects were instructed to return to the study site for additional visits as appropriate within the Screening period for completion of the required Screening assessments. The Investigator reviewed the results from all screening procedures, and determined if the subjects were eligible for enrollment into the study. The following procedures and evaluations were conducted at Screening: informed consent, medical and gynecologic history were taken; prior/concomitant medical information was collected; collection of vital signs (body temperature, heart rate (hereinafter, “HR”), respiratory rate (hereinafter, “RR”), and blood pressure (hereinafter, “BP”); height and body weight measurements were taken, and BMI was calculated; a physical exam was carried out; 12-lead ECG was carried out; pelvic and breast examination was carried out; a pap smear was done; blood and urine samples were collected for blood chemistry, hematology and coagulations tests; blood samples were collected for measurements of FSH (subjects with ≥12 months of spontaneous amenorrhea or bilateral oophorectomy were excluded), estradiol, estrone, progesterone, and TSH levels (note: if TSH was abnormal as per lab range, reflex testing of T3 and T4 was performed); a urine pregnancy test (subjects with history of tubal ligation, bilateral oophorectomy, or ≥55 years of age and amenorrheic for at least 1 year were excluded); mammogram was performed (if not completed within 6 months of Visit 1 or if a report was not available); endometrial biopsy was performed; dispensed Issue Treatment Hot Flush/Bleeding and Spotting diaries (minimum of 14 days prior to Visit 1); and instructions were given regarding the importance of the diary to the study, methods of completion and methods for returning the diary to the study site.

8. Double-Blind Treatment Phase

All on-therapy visits had a visit window to allow for slight variations in subject schedules and weekends; however, every effort was made to have the subject return on the designated study day. The visit window could be longer, but only with the approval of the Medical Monitor. When planning visits, the overall treatment period was maintained; with subsequent visits based on the Randomization day. Study visits were typically conducted so as to include the activities outlined in Table 23, above, and as set forth below.

Visit 1 (Week 0/Day 1)

Subjects who met the eligibility criteria were randomized into one of the treatment arms in the VMS Substudy or non-Substudy. Study medication were dispensed once all Screening Procedures had been completed and eligibility had been verified. The following procedures and evaluations were conducted at this visit: review of study entry criteria; update of medical and gynecological history (to record any new conditions or information that emerged during Screening); collection of vital signs (body temperature, HR, RR, and BP); distribution and collection of subject completed Menopause-Specific Quality of Life Questionnaire (hereinafter, “MENQOL form”) distribution and collection of subject completed the Medical Outcomes Study-Sleep Questionnaire (hereinafter, “MOS-Sleep form”); recording and documentation of adverse events (AEs) since the last visit; collection of prior/concomitant medication information since the last visit; collection of Screening diaries and assignment to VMS Substudy [a minimum of 14 consecutive days of complete hot flush diary data was required during the baseline assessment at Screening, and these consecutive days must have occurred within the 14 days prior to the Randomization visit (not counting the Randomization visit day itself); the most recent 7 consecutive days of data prior to randomization (Day −7 to Day −1) was used to determine the baseline number of mild, moderate and severe hot flushes for each subject] and non-Substudy [to participate in the VMS Substudy, a subject must have reported ≥7 moderate to severe hot flushes per day, or ≥50 per week, at the baseline assessment during Screening; subjects whose hot flushes were less frequent may still have participated as non-Substudy subjects until enrollment of the non-VMS Substudy was reached]; subjects were randomized to study medication; subjects were instructed on study medication self-administration and on completion of subject diary [Issue Treatment Hot Flush/Bleeding and Spotting diaries were handed out for the first 4 weeks of treatment (with allowances for the visit window)]; study medication was dispensed for the subsequent 4 weeks of treatment (with allowances for the visit window), and subjects were instructed to take it at bedtime with food; and subjects were further instructed to return to return to the study site in approximately 4 weeks (Day 28±3 d).

Visit 3 (Week 8/Day 56±3, Interim)

Subjects returned to the study site at Week 8 (Day 56±3 days) of treatment initiation. The following procedures and evaluations were conducted at this visit: collection of vital signs (body temperature, HR, RR, and BP); collection and review of completed subject diaries for the previous 4 weeks of treatment by site personnel; Issue Treatment Hot Flush/Bleeding and Spotting diaries were dispersed for the subsequent 4 weeks of treatment (with allowances for the visit window) and instructions for completion were reviewed, if necessary by site personnel; study medication was dispensed for the subsequent 4 weeks of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; for VMS Substudy subjects only, the CGI assessment was completed; and subjects were instructed to return to the study site in approximately 4 weeks (Week 12, Day 84±3 days).

Visit 4 (Week 12/Day 84±3, Interim)

Subjects returned to the study site at Week 12 (Day 84±3 days) of treatment initiation. The following procedures and evaluations were conducted at this visit: collection of vital signs (body temperature, HR, RR, and BP); body weight measurements were taken; blood sample was collected for blood chemistry, hematology and coagulations tests; blood sample was collected for measurements of estradiol, estrone, and progesterone levels; a urine analysis was done; collection and review of completed subject diaries for the previous 4 weeks of treatment by site personnel; Issue Treatment Hot Flush/Bleeding and Spotting diaries were dispersed for the subsequent 3 months of treatment (with allowances for the visit window) and instructions for completion were reviewed, if necessary by site personnel; study medication was dispensed for the subsequent 3 months of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; distribution and collection of subject completed MENQOL; distribution and collection of subject completed MOS-Sleep form; and for VMS Substudy subjects only, the CGI assessment was completed. Subjects were instructed to return to the study site in approximately 3 months (Month 6, Day 180±4 days).

Visit 5 (Month 6/Day 180±3, Interim)

Subjects returned to the study site at Month 6 (Day 180±3 days) of treatment initiation. The following procedures and evaluations were conducted at this visit: collection of vital signs (body temperature, HR, RR, and BP); body weight measurements were taken; pelvic and breast examination was carried out; blood sample was collected for blood chemistry, hematology and coagulations tests; blood sample was collected for measurements of estradiol and estrone levels; a urine analysis was done; collection and review of completed subject diaries for the previous 6 months of treatment by site personnel; Issue Treatment Hot Flush/Bleeding and Spotting diaries were dispersed for the subsequent 3 months of treatment (with allowances for the visit window) and instructions for completion were reviewed, if necessary by site personnel; study medication was dispensed for the subsequent 3 months of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; distribution and collection of subject completed MENQOL form); and distribution and collection of subject completed MOS-Sleep form. Subjects were instructed to return to the study site in approximately 3 months (Month 9, Day 270±4 days).

Visit 6 (Month 9/Day 270±3, Interim)

Subjects returned to the study site at Month 9 (Day 270±3 days) of treatment initiation. The following procedures and evaluations were conducted at this visit: collection of vital signs (body temperature, HR, RR, and BP); blood sample was collected for blood chemistry, hematology and coagulations tests; blood sample was collected for measurements of estradiol and estrone levels; a urine analysis was done; collection and review of completed subject diaries for the previous 3 months of treatment by site personnel; Issue Treatment Hot Flush/Bleeding and Spotting diaries were dispersed for the subsequent 3 months of treatment (with allowances for the visit window) and instructions for completion were reviewed, if necessary by site personnel; study medication was dispensed for the subsequent 3 months of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; and concomitant medication information since the last visit were collected. Subjects were instructed to return to the study site in approximately 3 months (Month 12, Day 360±4 days).

Visit 7 (Month 12/Day 360±4, End of Treatment)

Subjects returned to the study site at Month 12 (Day 360±4 days) of treatment initiation. The following procedures and evaluations were conducted at this visit: a physical exam was carried out; collection of vital signs (body temperature, HR, RR, and BP); body weight measurements were taken; pelvic and breast examination was carried out; a pap smear was done; mammography was done; 12-lead ECG was carried out; endometrial biopsy was done; blood sample was collected for blood chemistry, hematology and coagulations tests; blood sample was collected for measurements of estradiol, estrone, and progesterone levels; a urine analysis was done; collection and review of completed Issue Treatment Hot Flush/Bleeding and Spotting diaries for the previous 3 months of treatment by site personnel; all study medication containers and unused study medication from the previous period of treatment were collected by study personnel; treatment compliance was reviewed with the subject; all AEs since the last visit were recorded and documented; concomitant medication information since the last visit were collected; distribution and collection of subject completed MENQOL form; distribution and collection of subject completed MOS-Sleep form; and instructions were given for reporting of serious adverse events that occur within 30 days after the last dose of the study medication.

9. Post-Treatment Follow-Up

Each subject who received study medication received a follow-up phone call, regardless of the duration of therapy, approximately 15 days following the last dose of study medication. The follow-up generally took place after receipt of all safety assessments (e.g., endometrial biopsy and mammography results). The follow-up phone call included: review of ongoing adverse events and any new adverse events that occurred during the 15 days following the last dose of study medication; review of ongoing concomitant medications and any new concomitant medications that occurred during the 15 days following the last dose of study medication; discussion of all end of study safety assessments and determination if further follow up or clinic visit is required; and instructions were again provided for reporting of serious adverse events that occur within 30 days after the last dose of study medication.

D. Subject Identification and Randomization

Each subject was given a unique subject number at the start of Screening that was used to identify their clinical site and sequential number. In addition to the assigned subject number, subject initials were also used for identification.

Eligible subjects were randomized at Visit 1 (Week 0/Day 1). The randomization code was created using a computer-generated randomization schedule prepared by a statistician prior to the start of the study.

E. Test Product, Dose, and Mode of Administration

TX-001HR is an oval, opaque, pink, soft gelatin formulation of a combination product comprising of 17β-estradiol hemihydrate and micronized progesterone. Since two different sizes of capsules were necessary to accommodate the different doses, a double-dummy technique was used. The two sizes of placebo capsules were an identical match to the active study medication, but without the estrogen/progesterone. Study medication was packaged in blister packs, labeled and sent to the each site. The carton and packaging labels did not contain information that unblinds the identity of the medication.

Subjects that were randomized to active treatment self-administered orally one of the following four arms of active TX-001HR treatment daily at bedtime with food for 12 months.
Treatment 1: Combined Estradiol 1 mg/Progesterone 100 mg formulation [large active; small placebo]
Treatment 2: Combined Estradiol 0.5 mg/Progesterone 100 mg formulation [large active; small placebo]
Treatment 3: Combined Estradiol 0.5 mg/Progesterone 50 mg formulation [large placebo; small active]
Treatment 4: Combined Estradiol 0.25 mg/Progesterone 50 mg formulation [large placebo; small active]
   Two placebo gel capsules matching the test product were taken orally by subjects participating in the VMS Substudy that were randomized to placebo. In order to maintain the study blind, the study had a double blind, double dummy treatment. Subjects randomized to active treatment took a placebo gel capsule matching the alternate capsule size from their active treatment. All subjects took 1 large and 1 small capsule.
Treatment 5: Placebo
   [large placebo; small placebo]

All subjects self-administered orally two capsules daily at bedtime with food for 12 months. Each subject was dispensed enough study medication to last until the next scheduled visit, with allowances for visit windows. The subjects were instructed to return the used and unused containers of study medication in the original packaging to the study site at Visits 2, 3, 4, 5, 6 and 7. The sites verified and documented compliance based on counts of dispensed/returned study medication and any additional information reported by the subjects (e.g., regarding lost capsules).

Study medication were dispensed to all eligible subjects at Visits 1 to 6. At Visits 2 to 7 subjects were instructed to return all used study medication containers and any unused study medication to study personnel in the original packaging, and were dispensed with the new medication for the subsequent period.

If a subject discontinued study participation or was terminated from the study, the subject was instructed to return all study medication containers and any unused study medication at the time of discontinuation/terminations.

The first day that the study medication was administered by the subject was considered Day 1 and all subsequent visits were based on this day. Compliance was determined from the subject diary and pill count through week 12; during this time, the subject was instructed to record all administrations taken and missed. After the week 12 visit, study medication compliance was monitored through counts of capsules dispensed and returned as well as explanatory information reported by the subject. Each study subject was required to be at least 80% compliant with study medication, based on capsule count over each study visit interval to be considered compliant. If a subject was less than 80% compliant, the investigator was instructed to discuss withdrawing the subject with the Medical Monitor.

Subjects were instructed to return completed study diaries to study personnel at their visits. Study personnel reviewed the subject diary with the subject to ensure proper documentation of study medication dosing and other information recorded on the diary. Upon return of the study medication containers, study personnel were responsible for recording the amount of study medication returned, the amount of study medication used by the subject, and the amount of study medication unused by the subject on a drug accountability log.

F. Endpoints

1. Primary Efficacy Endpoint: Vasomotor Symptoms (VMS Substudy)

A minimum of 14 consecutive days of complete hot flush diary data was required during the baseline assessment at Screening, and these consecutive days had to occur within the last 14 days prior to the Randomization visit (not counting the Randomization visit day itself). The most recent 7 consecutive days of data prior to Randomization (Day −7 to Day −1) was used to determine the baseline number of mild, moderate and severe hot flushes for each subject. The number of moderate to severe hot flushes from these 7 days was also used to determine eligibility for the VMS Substudy.
Mean change in frequency of moderate to severe vasomotor symptoms from baseline to week 4 in an active treatment group compared with placebo.
Mean change in frequency of moderate to severe vasomotor symptoms from baseline to week 12 in an active treatment group compared with placebo.
Mean change in severity of moderate to severe vasomotor symptoms at baseline to mild, moderate to severe vasomotor symptoms at week 4 in an active treatment group compared with placebo.
Mean change in severity of moderate to severe vasomotor symptoms at baseline to mild, moderate to severe vasomotor symptoms at week 12 in an active treatment group compared with placebo.

2. Primary Safety Endpoint: Endometrial Hyperplasia
The primary safety endpoint was the incidence rate of endometrial hyperplasia at 12 months (to demonstrate a hyperplasia rate that is ≤1 percent with an upper bound of the one-sided 95 percent CI for that rate that does not exceed 4 percent) based on an a priori plan in which a consensus among two out of three pathologists is the final endometrial pathology diagnosis. When the two primary pathologists disagree on the presence of hyperplasia, the read of the third pathologist will be utilized.

For the primary endpoint, all endometrial biopsies were centrally read by three pathologists. Two pathologists designated by the sponsor, were considered to be the primary pathologists (the pathologists were blinded to this designation).

Each pathologist classified the biopsies into one of the following three categories:
Category 1: Non-endometrial malignancy/non-hyperplasia—includes proliferative endometrium, weakly proliferative endometrium, disordered proliferative pattern, secretory endometrium, endometrial tissue (other) [i.e., benign, inactive or atrophic fragments of endometrial epithelium, glands, stroma, etc.], endometrial tissue insufficient for diagnosis, no endometrium identified, no tissue identified, other.
Category 2: Endometrial hyperplasia—includes simple hyperplasia with or without atypia and complex hyperplasia with or without atypia.
Category 3: Endometrial malignancy.

The reads of the two primary pathologists were utilized. Consensus was reached when the two primary pathologist readers agreed on any of the above categories. For example, any 2 subcategories of “Non-endometrial malignancy/non-hyperplasia” will be classified as “Category 1: Non-endometrial malignancy/non-hyperplasia,” if the primary pathologists disagreed on the presence of hyperplasia, the result of the third pathologist was utilized and the final decision regarding the presence of hyperplasia was based on the diagnosis of the majority.

If all three readings are disparate (i.e., each falls into a different category—Category 1, 2, or 3), the final diagnosis was based on the most severe of the three readings.

A confidence interval approach was used to determine if the hyperplasia incidence rate was acceptable. For each active treatment group, the incidence rate of hyperplasia at year 1 and the associated upper 95% 1-sided confidence limit was to be calculated. An observed incidence rate of 1% or less with an upper 1-sided 95% confidence limit of 4% or less was considered acceptably low.

3. Secondary VMS Substudy Endpoints

Secondary VMS SubStudy Endpoints include the following:
Mean change in frequency of moderate to severe vasomotor symptoms from baseline to each week up to week 12.
Mean change in severity of moderate to severe vasomotor symptoms from baseline to mild, moderate to severe vasomotor symptoms each week up to week 12.
Mean change in frequency and severity of mild, moderate and severe vasomotor symptoms from baseline to each week up to week 12.
Percent of subjects with 50% and, separately, 75% reduction in moderate to severe vasomotor symptoms from baseline at each week up to week 12.
Percent treatment responders at weeks 4, 8 and 12 based on subject satisfaction with treatment (Clinical Global Impression [CGI]) compared to changes in frequency of moderate to severe vasomotor symptoms from baseline.

4. Secondary Endometrial Hyperplasia Endpoint

A supplemental secondary analysis was performed. The secondary endpoint was the incidence rate of endometrial hyperplasia at 12 months based on agreement of two of the three pathologists' reads. All biopsies were read by three blinded pathologists, and the results from the three pathologists were utilized. In this supplemental analysis, the final diagnosis was based on agreement of two of the three pathologist reads. Consensus was reached when two of the three pathologist readers agreed on any of the above categories. For example, any 2 subcategories of “Non-endometrial malignancy/non-hyperplasia” was classified as “Category 1: Non-endometrial malignancy/non-hyperplasia.” If all three readings were disparate (i.e., each falls into a different category—Category 1, 2, or 3), the final diagnosis was based on the most severe of the three readings.

5. Other Secondary Endpoints

Other secondary endpoints included the following:
Proportion of subjects with cumulative amenorrhea from day 1 to day 364
No bleeding: % by cycle and cumulative for consecutive cycles.
Number of days with bleeding/spotting.
Evaluation of frequency and severity of hot flushes over 12 weeks in the overall study subjects.
MENQOL evaluation parameters.
MOS-Sleep evaluation parameters.
Trough hormone assessment for serum estradiol and estrone.

The severity of vasomotor symptoms is defined clinically as follows:

| Severity | Description |
|---|---|
| Mild | Sensation of heat without sweating |
| Moderate | Sensation of heat with sweating, able to continue activity |
| Severe | Sensation of heat with sweating, causing cessation of activity |

6. Safety Endpoints

Vital signs, weight, changes in clinical laboratory measurements (including hematology, clinical chemistry, urinalysis, and Pap smear), and adverse events were evaluated as part of the safety endpoints. Changes in physical exam, ECG, pelvic exam, and mammogram were evaluated. The incidence of hyperplastic polyps and polyps associated atypia were considered in the safety review. The comparability of the active treatment groups with regard to the frequency and severity distribution of any adverse event reported by at least 5% of the subjects in either the treatment groups would be evaluated by comparing the frequency distributions of the 5 treatment groups.

G. Statistical Considerations

1. Randomization and Stratification

Subjects in the VMS Substudy were randomized within each study site to one of the treatment groups below in a 1:1:1:1:1 allocation ratio. Subjects not in the VMS Substudy were randomized to one of the active treatment groups in a 1:1:1:1 allocation ratio. Subjects were randomized to study medication within each site using a reproducible, computer-generated block randomization schedule. Randomization codes were generated and held with restricted access to decrease the chance of unblinding and to minimize bias.
Treatment 1: Combined Estradiol 1 mg/Progesterone 100 mg formulation
Treatment 2: Combined Estradiol 0.5 mg/Progesterone 100 mg formulation
Treatment 3: Combined Estradiol 0.5 mg/Progesterone 50 mg formulation
Treatment 4: Combined Estradiol 0.25 mg/Progesterone 50 mg formulation
Treatment 5: Placebo

2. Sample Size Rationale

The overall study sample size was based on the target that the combination therapy is effective at achieving a ≤1% incidence rate of endometrial hyperplasia following 12 months of therapy and that the upper bound of the 95% confidence interval of the estimated incidence rate is ≤4%. The VMS sub-study sample size was based on the expected changes in average weekly frequency and severity of vasomotor symptoms from baseline to weeks 4 and 12.

Overall Study:

With 250 subjects in each active treatment group completing 12 months of treatment and being successfully evaluated for endometrial hyperplasia at baseline and 12 months, two or fewer reports of endometrial hyperplasia would result in an annual incidence rate of ≤1% and an upper bound on a one-sided 95% confidence interval of ≤2.5% (exact binomial).

VMS Sub-study:

The primary method of analysis for both frequency counts and severity index was to account for missing information using imputation by last observation carried forward and a linear mixed effects covariance pattern model that treated subjects as a random effect and accounted for the repeated frequency and severity measures at baseline, week 4 and week 12. Each of the four active treatment groups and the 4 co-primary outcomes were compared to the placebo group in a hierarchical order to preserve the test level of significance for each comparison at 5% (two-sided). A two-group t-test was used to estimate sample size requirements.

Change in Frequency:

The mean changes from baseline in weekly frequency of moderate to severe hot flushes was assumed to be at least −56 for any given active treatment group and −35 for the placebo group at both weeks 4 and 12. A common, between subject standard deviation of 35 across treatment groups and weeks was further assumed.

Change in Severity Index:

The mean changes in the severity score from baseline for mild, moderate and severe hot flushes was assumed to be at least −0.7 for any given active treatment group and −0.4 for the placebo group at both weeks 4 and 12. A common, between subject standard deviation of 0.6 across treatment groups and weeks was further assumed.

Enrolling 150 subjects in each treatment group and allowing for up to 20% of the subjects in each group being ineligible for the primary analyses provides at least 90% power to test the primary VMS hypotheses.

3. Datasets to be Analyzed

Population datasets for analyses are defined below for the estimation of endometrial hyperplasia in the active treatment groups, for the comparison between active treatment groups and the placebo group of the change from baseline in the weekly frequency and severity of moderate to severe hot flushes in the VMS Sub-study, and for the overall safety evaluation.
All Randomized: All subjects that are randomized into the study.
All Treated/Safety: All randomized subjects that took at least one application of study treatment.
Endometrial Hyperplasia (EH): All Treated subjects randomized to an active treatment group who remain on study treatment for 12 months, no major protocol violations, and have a biopsy at baseline and month 12 that can be evaluated for the presence of endometrial hyperplasia. In addition, if endometrial hyperplasia is diagnosed at a mid-study visit in a subject who had a definitive biopsy at baseline, the subject with the endometrial hyperplasia event will be counted in calculating the annual incidence.
VMS Sub-study Modified Intent-to-Treat (MITT): All Treated subjects who qualify for the VMS Sub-study, had baseline measurement of frequency and severity of moderate to severe hot flushes, and had at least one week of reporting of frequency and severity of hot flushes following initiation of study treatment;
VMS Sub-study Efficacy Evaluable (EE): MITT subjects who were correctly included at randomization for efficacy reasons and who had all 4-week and 12-week efficacy evaluation data.

Efficacy analyses for endometrial hyperplasia will be performed using the EH population, efficacy analyses for VMS will be performed using the MITT and EE populations, and safety analyses will be performed using the All Treated/Safety population.

Other analyses of spotting and bleeding, flushing experience in non-Substudy subjects, MENQOL and MOS-Sleep responses will rely on available data.

Subjects with major deviations from the protocol as specified in the Statistical Analysis Plan will be excluded from the analyses.

4. Analyses

Primary Efficacy Analyses—VMS

The weekly number of moderate to severe hot flushes and the weekly severity score were assessed as mean changes from baseline to weeks 4 and 12 for each subject. The weekly number of moderate to severe hot flushes for each assessment week (baseline, and weeks 4 and 12) were derived as:
Weekly Frequency=(total number of moderate and severe hot flushes for the 7 days of the subject week)
The weekly severity of hot flushes for each assessment week (baseline, and weeks 4 and 12) will be derived as:
   Weekly Severity Score=[(number of mild hot flushes for 7 days)×1+(number of moderate hot flushes for 7 days)×2+(number of severe hot flushes for 7 days)×3]/(total number of hot flushes over 7 days).

A weekly severity score of zero (0) will be assigned for subjects reporting no hot flushes for a given assessment week.

Absolute, changes from baseline and respective differences from placebo in frequency and severity of vasomotor symptoms were listed and summarized. Means, standard deviations (S.D.), minimum (MIN) and maximum (MAX) were provided for all 4 co-primary endpoints.

Mean changes from baseline in frequency and severity of vasomotor symptoms was assessed using a linear mixed effects covariance pattern model with baseline as a covariate, treatment (active vs. placebo), week (4 and 12) and treatment by week interaction. Subject was included in the model as a random effect and a compound symmetry model was assumed. A test for an overall treatment effect was assessed, as well as, weekly comparisons of active to placebo. A separate mixed model was run for each active dose comparing back to placebo. Statistical significance was declared if p<0.05 for each dose comparison of each of the 4 co-primary endpoints. The highest estradiol dose (1 mg) was tested first for each of the 4 co-primary endpoints and all endpoints should reach statistical significance compared to placebo prior to moving to the next test. The second test was a three-way comparison of the two 0.5 mg doses and placebo; if each of the 4 co-primary endpoints demonstrate statistical significance then separate pairwise comparisons would be made between each 0.5 mg doses and placebo. Finally, if all pairwise comparisons with placebo of the 0.5 mg doses were statistically significant then the 0.5 mg dose was compared to placebo. Ninety five percent (95%), two-sided confidence intervals were derived for changes from baseline and respective differences from placebo, and graphically displayed for each dose and week.

### Primary Safety Analysis-Endometrial Hyperplasia

The primary safety endpoint was the incidence rate of endometrial hyperplasia at 12 months (to demonstrate a hyperplasia rate that is ≤1 percent with an upper bound of the one-sided 95 percent CI for the rate that does not exceed 4 percent) based on an a priori plan in which a consensus among two out of three pathologists was the final endometrial pathology diagnosis. When the two primary pathologists disagreed on the presence of hyperplasia, the read of the third pathologist was utilized.

For the primary endpoint, all endometrial biopsies were centrally read by three pathologists. Two pathologists, designated by the sponsor, were considered to be the primary pathologists (the pathologists were blinded to this designation).

Each pathologist classified the biopsies into one of the following three categories:
Category 1: Non-endometrial malignancy/non-hyperplasia (includes proliferative endometrium, weakly proliferative endometrium, disordered proliferative pattern, secretory endometrium, endometrial tissue (other) [i.e., benign, inactive or atrophic fragments of endometrial epithelium, glands, stroma, etc.], endometrial tissue insufficient for diagnosis, no endometrium identified, no tissue identified, other.
Category 2: Endometrial hyperplasia—includes simple hyperplasia with or without atypia and complex hyperplasia with or without atypia.
Category 3: Endometrial malignancy.

For the primary analysis:

The reads of the two primary pathologists were utilized. Consensus was reached when both of the primary pathologist readers agreed on any of the above categories. For example, any 2 subcategories of “Non-endometrial malignancy/non-hyperplasia” were classified as “Category 1: Non-endometrial malignancy/non-hyperplasia”; if the primary pathologists disagreed on the presence of hyperplasia, the result of the third pathologist was utilized and the final decision regarding the presence of hyperplasia was based on the diagnosis of the majority.

If all three readings were disparate (i.e., each falls into a different category—Category 1, 2, or 3), the final diagnosis was based on the most severe of the three readings.

A confidence interval approach was used to determine if the hyperplasia incidence rate was acceptable. For each active treatment group, the incidence rate of hyperplasia at year 1 and the associated upper 95% 1-sided confidence limit was calculated. The primary analysis population for endometrium hyperplasia was endometrial hyperplasia (EH) population. A EH subject at year 1 is one who is randomly assigned and takes at least 1 dose of study medication, with no exclusionary protocol violation (as detailed at the Statistical Analysis Plan) and has a pretreatment endometrial biopsy and a biopsy at year 1, or who has developed endometrial hyperplasia at any time during the study. Any biopsy performed within the month 11 through month 13 (relative to the first day on study medication) will be considered a year 1 biopsy, and it will be included in the analysis as long as it was done within 30 days of the last dose of study medication.

The incidence rate of endometrial hyperplasia at year 1 was calculated as follows:

I=A/B

where I=incidence rate at 1 year evaluation,
A=all subjects with biopsies positive for endometrial hyperplasia during the study,
B=all subjects with biopsies during months 11 through 13 meeting the criteria specified above, plus all subjects with biopsies positive for endometrial hyperplasia by any of the pathologist before month 11.

An observed incidence rate of 1% or less with an upper 1-sided 95% confidence limit of 4% or less will be considered acceptably low.

In addition, 95% 2-sided confidence intervals were calculated for pairwise differences between groups in hyperplasia incidence rates.

Secondary VMS Analyses

Similar to the co-primary endpoints for weeks 4 and 12, assessments were made for changes in frequency and severity of mild, moderate and severe vasomotor symptoms for each assessment week up to week 12. Two assessments were made with one including only moderate and severe vasomotor symptoms and one including mild moderate and severe vasomotor symptoms. Severity scores were set to zero (0) for subjects reporting no moderate or severe flushes for the moderate and severe assessment, and set to zero (0) for subjects reporting no hot flushes for the mild moderate and severe assessments. Additionally, percent reductions from baseline and respective differences from placebo were reported.

The percent of treatment responders was identified within each treatment arm and compared (active treatments to placebo) at weeks 4, 8, and 12 by using the methodology described by Gerlinger et al., supra, that combines subject self-assessment of satisfaction with treatment (using the CGI) and changes in reported moderate to severe flushes. In particular, the Gerlinger approach first stratifies subjects' change from baseline in the number of moderate to severe hot flushes by their CGI response (7 levels ranging from “very much worse” to “very much better”); then discriminant analysis is applied to identify minimally clinically important reductions in the number of hot flushes that study subjects perceive as beneficial and that are then used to define a responder.

A second approach to identifying treatment responders was to calculate the percent of subjects with 50% and, separately, 75% reduction from baseline in moderate to severe vasomotor symptoms at each week up to week 12 and compare between active and placebo treatments.

Secondary Endometrial Hyperplasia Analysis

A supplemental secondary analysis was performed. The results from the three pathologists were utilized. In this supplemental analysis the final diagnosis was based on agreement of two of the three pathologist reads. Consensus was reached when two of the three pathologist readers agreed on any of the above categories. For example, any 2 subcategories of “Non-endometrial malignancy/non-hyperplasia” was classified as “Category 1: Non-endometrial malignancy/non-hyperplasia.” If all three readings were disparate (i.e., each fell into a different category—Category 1, 2, or 3), the final diagnosis was based on the most severe of the three readings.

Other Analyses

Other analyses included the following:
Proportion of subjects with cumulative amenorrhea from day 1 to day 364.
No bleeding: % by cycle and cumulative for consecutive cycles.
Number of days with bleeding/spotting.

Percent amenorrhea: Amenorrhea is defined as absence of bleeding or spotting. Within each treatment arm, the portion of subjects with cumulative amenorrhea from day 1 to day 364 was calculated and compared between active and placebo treatments.

Percent no bleeding: No bleeding is defined as absence of bleeding. Within each treatment arm, the percent of subjects with no bleeding was calculated by cycle and for consecutive cycles and compared between active and placebo treatments.

Number of days with bleeding/spotting: The number of days with bleeding/spotting, as reported on subject diaries, was summarized by cycle and treatment group.

Changes in frequency and severity of hot flushes over 12 weeks in non-Substudy subjects: Hot flush experience in the non-Substudy group (all actively treated subjects) was evaluated in the manner described earlier for the secondary analysis of the VMS Substudy data.

MENQOL: The menopause-specific quality of life questionnaire assesses changes in quality of life of study subjects over a one-month period. It was self-administered and was measured at baseline and at week 12, month 6 and month 12 during the study. It is composed of 29 questions distributed across 4 domains: vasomotor, psychosocial, physical and sexual. There is no total score so domain scores were analyzed separately. Change in monthly scores were summarized and described within each treatment group.

MOS-Sleep: The Medical Outcomes Study Sleep self-report questionnaire has 12 items that measure six dimensions of sleep over the past four weeks. It was self-administered and was measured at baseline and was measured at baseline and at week 12, month 6 and month 12 during the study. Change in scores over the past four weeks (total and subscales) was analyzed within each treatment.

Serum Concentrations of Estradiol, Estrone, and Progesterone

Serum concentrations of estradiol and estrone obtained at Screening and at Visit 2 (week 4), Visit 4 (week 12), Visit 5 (month 6), Visit 6 (month 9) and Visit 7 (month 12) and serum concentrations of progesterone obtained at Screening and at Visit 4 (week 12) and Visit 7 (month 12) were summarized by treatment group.

A summary of the mean serum concentration of estradiol for each time point and by treatment group for the Safety population, representing steady state serum concentrations for estradiol, is shown in Table 25. The overall mean estradiol concentration at Screening was 6.1 pg/mL and the median estradiol concentration was 4.4 pg/mL, consistent with postmenopausal status. The lower limit of quantification for estradiol was 2.00 pg/mL. A dose response was observed for serum concentrations of estradiol and estradiol levels remained consistent over time for each respective treatment arm.

A summary of the mean serum concentration of estrone for each time point and by treatment group for the Safety population, representing steady state serum concentrations for estrone, is shown in Table 26. The overall mean estrone concentration at Screening was 23.3 pg/mL and the median concentration was 20.8 pg/mL, consistent with postmenopausal ranges. The lower limit of quantification for estrone was 5.00 pg/mL. Generally, estrone levels were related in a dose dependent manner to the estradiol dose given; levels remained consistent over time for each respective treatment arm.

A summary of the mean serum concentration of progesterone for each time point and by treatment group for the Safety population, representing steady state serum concentrations for progesterone, is shown in Table 27. The overall mean progesterone concentration at Screening was 57.6 pg/mL and the median concentration was 50 pg/mL. The lower limit of quantification for progesterone was 50.0 pg/mL. A dose response was observed for serum concentrations of progesterone and progesterone levels remained consistent over time for each respective treatment arm.

In summary, throughout the study, a dose response was observed for serum concentrations of estradiol and for estrone related to the dose dependent manner of the estradiol dose given. Estradiol and estrone levels remained consistent over time for each respective treatment arm, and were similar in the 0.5 mg E/100 mg P and 0.5 mg E2/50 mg P groups. Additionally, a dose response was observed for serum concentrations of progesterone and progesterone levels remained consistent over time for each respective arm.

TABLE 25
Summary of Serum Concentrations of Estradiol at Screening
and Visits 2, 4, 5, 6, and 7 for Safety Population

| | 1 mg E2/ | 0.5 mg E2/ | 0.5 mg E2/ | 0.25 mg E2/ | |
|---|---|---|---|---|---|
| Estradiol | 100 mg P | 100 mg P | 50 mg P | 50 mg P | Placebo |
| (pg/mL) | (N = 415) | (N = 424) | (N = 421) | (N = 424) | (N = 151) |
| Screening | 415 | 423 | 421 | 421 | 150 |
| Mean (SD) | 6.28 (6.623) | 6.45 (7.235) | 5.75 (6.060) | 6.29 (6.247) | 5.63 (4.320) |
| Min, Max | 2.0, 67.2 | 2.0, 55.3 | 2.0, 67.3 | 2.0, 56.3 | 2.0, 25.2 |
| Median | 4.6 | 4.5 | 4.2 | 4.5 | 4.0 |
| Week 4 | 382 | 394 | 405 | 402 | 130 |
| Mean (SD) | 42.49 (36.506) | 23.03 (23.906) | 24.88 (25.429) | 18.50 (33.337) | 8.27 (22.495) |
| Min, Max | 2.0, 245.0 | 2.0, 268.0 | 2.0, 241.0 | 2.0, 403.0 | 2.0, 200.0 |
| Median | 34.5 | 18.6 | 18.7 | 12.2 | 3.8 |
| Week 12 | 352 | 365 | 374 | 371 | 117 |
| Mean (SD) | 44.46 (39.110) | 26.52 (27.316) | 26.75 (31.046) | 16.59 (19.258) | 8.54 (23.224) |
| Min, Max | 2.0, 376.0 | 2.0, 269.0 | 2.0, 292.0 | 2.0, 213.0 | 2.0, 238.0 |
| Median | 36.0 | 20.4 | 20.1 | 12.3 | 3.9 |
| Month 6 | 315 | 333 | 338 | 323 | 102 |
| Mean (SD) | 45.58 (49.032) | 24.23 (22.052) | 24.16 (16.533) | 16.61 (16.962) | 5.35 (4.677) |
| Min, Max | 2.0, 478.0 | 2.0, 230.0 | 2.0, 110.0 | 2.0, 164.0 | 2.0, 25.8 |
| Median | 35.9 | 20.2 | 20.9 | 13.2 | 4.2 |
| Month 9 | 292 | 318 | 320 | 296 | 95 |
| Mean (SD) | 44.46 (35.665) | 27.37 (35.265) | 24.56 (20.397) | 15.06 (13.547) | 7.99 (15.176) |
| Min, Max | 2.3, 248.0 | 2.0, 406.0 | 2.0, 182.0 | 2.0, 167.0 | 2.0, 97.5 |
| Median | 36.0 | 20.5 | 20.3 | 12.6 | 4.1 |
| Month 12 | 282 | 301 | 311 | 280 | 91 |
| Mean (SD) | 42.29 (41.206) | 24.60 (26.442) | 23.66 (18.646) | 15.23 (20.076) | 5.73 (7.278) |
| Min, Max | 2.0, 483.0 | 2.0, 295.0 | 2.0, 146.0 | 2.0, 243.0 | 2.0, 63.2 |
| Median | 35.2 | 20.4 | 20.1 | 12.2 | 4.1 |

TABLE 26
Summary of Serum Concentrations of Estrone at Screening
and Visits 2, 4, 5, 6, and 7 for Safety Population

| | 1 mg E2/ | 0.5 mg E2/ | 0.5 mg E2/ | 0.25 mg E2/ | |
|---|---|---|---|---|---|
| Estrone | 100 mg P | 100 mg P | 50 mg P | 50 mg P | Placebo |
| (pg/mL) | (N = 415) | (N = 424) | (N = 421) | (N = 424) | (N = 151) |
| Screening | 415 | 422 | 421 | 421 | 150 |
| Mean (SD) | 23.32 (12.590) | 23.31 (12.052) | 22.75 (12.958) | 23.60 (11.018) | 23.38 (11.148) |
| Min, Max | 5.0, 139.0 | 5.0, 98.9 | 5.0, 128.0 | 5.0, 60.7 | 6.6, 71.7 |
| Median | 21.2 | 20.6 | 20.3 | 21.3 | 20.8 |
| Week 4 | 382 | 394 | 405 | 402 | 130 |
| Mean (SD) | 213.79 (158.984) | 113.59 (72.335) | 119.50 (83.787) | 69.02 (39.823) | 23.78 (18.475) |
| Min, Max | 7.8, 1430.0 | 5.0, 485.0 | 5.4, 951.0 | 5.0, 274.0 | 5.0, 159.0 |
| Median | 188.5 | 103.0 | 111.0 | 62.8 | 19.1 |
| Week 12 | 352 | 365 | 373 | 373 | 117 |
| Mean (SD) | 227.31 (168.122) | 127.93 (81.185) | 125.56 (94.267) | 69.75 (38.198) | 25.59 (17.174) |
| Min, Max | 8.9, 1820.0 | 5.0, 582.0 | 5.6, 981.0 | 5.0, 213.0 | 5.0, 115.0 |
| Median | 211.0 | 114.0 | 108.0 | 63.9 | 21.3 |
| Month 6 | 315 | 334 | 338 | 323 | 103 |
| Mean (SD) | 235.03 (176.290) | 128.56 (93.074) | 128.26 (77.033) | 73.43 (43.900) | 24.15 (14.125) |
| Min, Max | 10.4, 1440.0 | 5.0, 876.0 | 9.3, 439.0 | 5.0, 360.0 | 5.0, 124.0 |
| Median | 209.0 | 116.5 | 120.0 | 67.2 | 22.3 |
| Month 9 | 293 | 318 | 320 | 296 | 95 |
| Mean (SD) | 241.57 (185.724) | 126.05 (88.286) | 132.08 (83.538) | 72.92 (41.457) | 29.84 (44.676) |
| Min, Max | 9.3, 1850.0 | 6.0, 800.0 | 5.0, 490.0 | 6.5, 354.0 | 5.0, 424.0 |
| Median | 212.0 | 115.5 | 122.5 | 68.7 | 22.0 |
| Month 12 | 283 | 302 | 311 | 280 | 90 |
| Mean (SD) | 227.80 (188.091) | 119.59 (78.141) | 127.60 (93.805) | 72.48 (46.474) | 28.32 (34.808) |
| Min, Max | 5.0, 1360.0 | 5.0, 450.0 | 6.1, 586.0 | 8.4, 352.0 | 6.8, 322.0 |
| Median | 195.0 | 114.5 | 112.0 | 66.2 | 22.0 |

TABLE 27
Summary of Serum Concentrations of Progesterone at Screening and Visits 4 and 7 for Safety Population

| | 1 mg E2/ | 0.5 mg E2/ | 0.5 mg E2/ | 0.25 mg E2/ | |
|---|---|---|---|---|---|
| Progesterone | 100 mg P | 100 mg P | 50 mg P | 50 mg P | Placebo |
| (pg/mL) | (N = 415) | (N = 424) | (N = 421) | (N = 424) | (N = 151) |
| Screening | 415 | 422 | 420 | 419 | 150 |
| Mean (SD) | 55.86 (24.393) | 65.35 (150.258) | 57.43 (52.544) | 55.99 (22.564) | 53.38 (11.325) |
| Min, Max | 50.0, 287.0 | 50.0, 3050.0 | 50.0, 1050.0 | 50.0, 264.0 | 50.0, 142.0 |
| Median | 50.0 | 50.0 | 50.0 | 50.0 | 50.0 |
| Week 12 | 351 | 366 | 374 | 373 | 117 |
| Mean (SD) | 451.68 (621.921) | 547.83 (1884.845) | 228.52 (618.660) | 247.17 (441.289) | 57.32 (30.769) |
| Min, Max | 50.0, 6770.0 | 50.0, 29000.0 | 50.0, 10200.0 | 50.0, 4730.0 | 50.0, 292.0 |
| Median | 284.0 | 250.0 | 123.5 | 132.0 | 50.0 |
| Month 12 | 283 | 301 | 311 | 280 | 91 |
| Mean (SD) | 534.41 (1375.196) | 386.53 (781.045) | 181.41 (242.823) | 219.11 (678.268) | 56.26 (20.231) |
| Min, Max | 50.0, 18800.0 | 50.0, 10500.0 | 50.0, 2240.0 | 50.0, 10300.0 | 50.0, 183.0 |
| Median | 263.0 | 232.0 | 119.0 | 115.0 | 50.0 |

Safety and Tolerability Analyses

Safety and tolerability were assessed by summarizing the incidence, relatedness, severity, and type of adverse events and treatment-emergent changes in safety evaluation criteria. Safety evaluation results were listed for all subjects and summaries will be tabulated by treatment. All safety and tolerability analyses were descriptive and used the All Treated/Safety population.
Adverse Events: The number (percentage) of subjects with at least one treatment-emergent AE will be presented in a frequency table by MedDRA system-organ class and per MedDRA “preferred” term. A similar summary will be created for SAEs and AEs resulting in interruption of treatment or leading to discontinuation of study medication. Summaries will also be presented by severity and relationship to study medication.
Vital Signs, Weight, ECGs, Mammograms, and Clinical Laboratory Values: Change from baseline will be summarized over time and any abnormal values considered clinically significant will be tabulated. Shift tables will be generated.
Physical Examinations: Changes from baseline to final evaluation will be categorized as improved, no change, or worsened for each body system. The number and percentage of subjects in each category of change will be given at final evaluation for each treatment group and body system.

G.6.5 Treatment Exposure and Compliance

Duration of exposure to study medication and compliance with administration of study medication were summarized by treatment group. Compliance was estimated as the percent of study medication actually used compared to the theoretical amount of drug that could have been used during each subject's exposure to study medication. No adjustments were made for missed doses or interruption of study medication.

H. Efficacy Results and Analysis

The Replenish Trial studied TX-001HR, an investigational bio-identical hormone therapy combination of 17β-estradiol and natural progesterone in a single, oral softgel, for the treatment of moderate to severe vasomotor symptoms (VMS) due to menopause in post-menopausal women with an intact uterus.

As explained above, the Replenish Trial evaluated four doses of TX-001HR and placebo in 1,835 post-menopausal women between 40 and 65 years old. The doses studied were as follows:

17β-estradiol 1 mg/progesterone 100 mg (n=416)

17β-estradiol 0.5 mg/progesterone 100 mg (n=423)

17β-estradiol 0.5 mg/progesterone 50 mg (n=421)

17β-estradiol 0.25 mg/progesterone 50 mg (n=424)

Placebo (n=151)

The results of the Replenish Trial demonstrated:
TX-001HR estradiol 1 mg/progesterone 100 mg and TX-001HR estradiol 0.5 mg/progesterone 100 mg both achieved all four of the co-primary efficacy endpoints and the primary safety endpoint (see, Tables 28-31).
TX-001HR of estradiol 1 mg/progesterone 100 mg and TX-001HR estradiol 0.5 mg/progesterone 100 mg both demonstrated a statistically significant and clinically meaningful reduction from baseline in both the frequency and severity of hot flashes compared to placebo (see, Tables 28-31 and FIGS. 5-6).
TX-001HR estradiol 0.5 mg/progesterone 50 mg and TX-001HR estradiol 0.25 mg/progesterone 50 mg were not statistically significant at all of the co-primary efficacy endpoints (see, Tables 28-31). The estradiol 0.25 mg/progesterone 50 mg dose was included in the clinical trial as a non-effective dose to meet the requirement of the FDA guidance to identify the lowest effective dose.
The incidence of consensus endometrial hyperplasia or malignancy was 0% across all four TX-001HR doses.

The Replenish study also demonstrated a dose response favoring the higher doses of estradiol in combination with progesterone. Importantly, multiple doses of TX-001HR allow for the ability to individualize therapy to meet the needs of a diverse population of women.

The most common adverse events (>5%) reported in the active treatment groups were breast tenderness, headache, nasopharyngitis, abdominal pain, upper respiratory infection, nausea, and back pain. Surprisingly, there was a very low reported incidence of adverse events of somnolence with TX-001HR, in contrast to commercially available oral progesterone (such as the reference listed drug Prometrium) where somnolence has been reported as a significant side effect. There were no unexpected safety signals.

TX-001HR estradiol 1 mg/progesterone 100 mg and estradiol 0.5 mg/progesterone 100 mg were also associated with clinically meaningful and statistically significant improvements in secondary endpoints including:
Menopause-Specific Quality of Life (MENQOL), a self-reported quality of life measure that had statistically significant improvements in the total score and vasomotor symptoms domain (see, Tables 32-35);
Clinical Global Impression (CGI) scale, a well-established patient reported outcome tool that showed a statistically significant and clinically meaningful improvement (see, Table 36); and
Responder analysis of the reduction of hot flashes of greater than 50% and 75% were statistically significant in the active treatment groups as compared to placebo (see, Table 37, below).

TABLE 28
Change From Baseline to Weeks 4 and 12 in Frequency Of Moderate To Severe Vasomotor Symptoms-MMRM
(MITT-VMS Population)

| | TRT1 (N = 141) | | TRT2 (N = 149) | | TRT3 (N = 147) | | TRT4 (N = 154) | | Placebo (N = 135) | |
|---|---|---|---|---|---|---|---|---|---|---|
| | | Change | | Change | | Change | | Change | | Change |
| | Actual | From | Actual | From | Actual | From | Actual | From | Actual | From |
| Study Week | Value | Baseline | Value | Baseline | Value | Baseline | Value | Baseline | Value | Baseline |
| Baseline | | | | | | | | | | |
| N | 141 | | 149 | | 147 | | 154 | | 135 | |
| Mean (SD) | 74.4 | | 72.1 | | 75.9 | | 77.0 | | 72.4 | |
| | (35.26) | | (27.76) | | (28.04) | | (30.42) | | (23.26) | |
| Min, Max | 0, 331 | | 38, 215 | | 29, 190 | | 32, 208 | | 10, 155 | |
| Median | 66 | | 62 | | 66 | | 65 | | 66 | |
| Week 4 | | | | | | | | | | |
| N | 134 | 134 | 144 | 144 | 142 | 142 | 152 | 152 | 126 | 126 |
| Mean (SD) | 31.5 | −40.6 | 37.2 | −35.1 | 41.5 | −33.6 | 38.4 | −38.9 | 45.9 | −26.4 |
| | (29.45) | (30.59) | (26.68) | (29.14) | (33.85) | (30.64) | (32.79) | (31.04) | (27.52) | (27.05) |
| Min, Max | 0, 161 | −140, 51 | 0, 115 | −177, 23 | 0, 233 | −140, 105 | 0, 207 | −125, 41 | 0, 164 | −122, 80 |
| Median | 23 | −41 | 35 | −32 | 34 | −35 | 30 | −36 | 41 | −24 |
| Difference | | | | | | | | | | |
| from | | | | | | | | | | |
| Placebo[1] | | | | | | | | | | |
| LS Mean | | −12.81 | | −8.07 | | −4.81 | | −10.40 | | |
| Standard | | 3.30 | | 3.25 | | 3.26 | | 3.22 | | |
| Error | | | | | | | | | | |
| 95% CI | | (−19.29, | | (−14.46, | | (−11.21, | | (−16.73, | | |
| | | −6.32) | | −1.68) | | 1.59) | | −4.08) | | |
| P-value | | <.001 | | 0.013 | | 0.141 | | 0.001 | | |
| Week 12 | | | | | | | | | | |
| N | 124 | 124 | 129 | 129 | 124 | 124 | 135 | 135 | 115 | 115 |
| Mean (SD) | 17.1 | −55.1 | 19.1 | −53.7 | 25.2 | −50.2 | 24.1 | −52.4 | 32.0 | −40.2 |
| | (20.65) | (31.36) | (21.87) | (31.93) | (27.45) | (31.35) | (28.42) | (33.90) | (26.24) | (29.79) |
| Min, Max | 0, 84 | −207, 19 | 0, 92 | −177, 26 | 0, 129 | −148, 20 | 0, 178 | −159, 68 | 0, 131 | −155, 38 |
| Median | 9 | −53 | 14 | −51 | 16 | −51 | 16 | −51 | 30 | −40 |
| Difference | | | | | | | | | | |
| from | | | | | | | | | | |
| Placebo[1] | | | | | | | | | | |
| LS Mean | | −16.58 | | −15.07 | | −10.79 | | −11.71 | | |
| Standard | | 3.44 | | 3.39 | | 3.41 | | 3.36 | | |
| Error | | | | | | | | | | |
| 95% CI | | (−23.33, | | (−21.72, | | (−17.48, | | (−18.31, | | |
| | | −9.82) | | −8.42) | | −4.10) | | −5.11) | | |
| P-value | | <.001 | | <.001 | | 0.002 | | <.001 | | |

[1]Derived from the MMRM model with Treatment, Week (1-12), Treatment-by-Week interaction as factors, Baseline as covariate, and Subject as repeated measures unit. Difference is estimated from the simple contrast between the least squares means.

TABLE 29
Change From Baseline to Weeks 4 and 12 in Frequency Of Moderate To Severe Vasomotor Symptoms-LOCF
ANCOVA (MITT-VMS Population)

| | TRT1 (N = 141) | | TRT2 (N = 149) | | TRT3 (N = 147) | | TRT4 (N = 154) | | Placebo (N = 135) | |
|---|---|---|---|---|---|---|---|---|---|---|
| | | Change | | Change | | Change | | Change | | Change |
| | Actual | From | Actual | From | Actual | From | Actual | From | Actual | From |
| Study Week | Value | Baseline | Value | Baseline | Value | Baseline | Value | Baseline | Value | Baseline |
| Baseline | | | | | | | | | | |
| N | 141 | | 149 | | 147 | | 154 | | 135 | |
| Mean (SD) | 74.4 | | 72.1 | | 75.9 | | 77.0 | | 72.4 | |
| | (35.26) | | (27.76) | | (28.04) | | (30.42) | | (23.26) | |
| Min, Max | 0, 331 | | 38, 215 | | 29, 190 | | 32, 208 | | 10, 155 | |
| Median | 66 | | 62 | | 66 | | 65 | | 66 | |
| Week 4 | | | | | | | | | | |
| N | 141 | 141 | 149 | 149 | 147 | 147 | 154 | 154 | 135 | 135 |
| Mean (SD) | 35.4 | −39.0 | 37.7 | −34.4 | 43.4 | −32.5 | 38.3 | −38.7 | 45.8 | −26.6 |
| | (37.79) | (30.97) | (26.45) | (28.93) | (35.76) | (30.72) | (32.59) | (30.89) | (27.23) | 7.31) |
| Min, Max | 0, 294 | −140, 51 | 0, 115 | −177, 23 | 0, 233 | −140, 105 | 0, 207 | −125, 41 | 0, 164 | −122, 80 |
| Median | 25 | −40 | 36 | −31 | 34 | −34 | 30 | −36 | 41 | −22 |
| Difference | | | | | | | | | | |
| from Placebo[1] | | | | | | | | | | |
| LS Mean | | −11.60 | | −7.97 | | −4.50 | | −10.19 | | |
| Standard Error | | 3.28 | | 3.23 | | 3.25 | | 3.21 | | |
| 95% CI | | (−18.04, | | (−14.32, | | (−10.88, | | (−16.50, | | |
| | | −5.17) | | −1.62) | | 1.87) | | −3.89) | | |
| P-value | | <.001 | | 0.014 | | 0.166 | | 0.002 | | |
| Week 12 | | | | | | | | | | |
| N | 141 | 141 | 149 | 149 | 147 | 147 | 154 | 154 | 135 | 135 |
| Mean (SD) | 21.0 | −53.4 | 20.0 | −52.0 | 27.3 | −48.6 | 26.3 | −50.7 | 36.0 | −36.4 |
| | (34.96) | (32.93) | (22.61) | (30.85) | (31.01) | (33.25) | (30.67) | (34.06) | (30.41) | (31.95) |
| Min, Max | 0, 294 | −207.22 | 0, 92 | −177, 26 | 0, 171 | −148, 49 | 0, 178 | −159, 68 | 0, 164 | −155, 80 |
| Median | 9 | −53 | 14 | −51 | 18 | −50 | 18 | −51 | 33 | −33 |
| Difference | | | | | | | | | | |
| from Placebo[1] | | | | | | | | | | |
| LS Mean | | −15.83 | | −15.84 | | −10.12 | | −11.60 | | |
| Standard Error | | 3.32 | | 3.28 | | 3.29 | | 3.26 | | |
| 95% CI | | (−22.35, | | (−22.27, | | (−16.58, | | (−17.99, | | |
| | | −9.31) | | −9.41) | | −3.66) | | −5.21) | | |
| P-value | | <.001 | | <.001 | | 0.002 | | <.001 | | |

[1]Derived from the ANCOVA model with Treatment as factor and Baseline as covariate.
Difference is estimated from the simple contrast between the least squares means.

TABLE 30
Change From Baseline to Weeks 4 and 12 in Severity Of Moderate To Severe Vasomotor Symptoms-MMRM
(MITT-VMS Population)

| | | Change | | Change | | Change | | Change | | Change |
|---|---|---|---|---|---|---|---|---|---|---|
| | Actual | From | Actual | From | Actual | From | Actual | From | Actual | From |
| Study Week | Value | Baseline | Value | Baseline | Value | Baseline | Value | Baseline | Value | Baseline |
| Baseline | | | | | | | | | | |
| N | 141 | | 149 | | 147 | | 154 | | 135 | |
| Mean (SD) | 2.54 | | 2.51 | | 2.50 | | 2.51 | | 2.52 | |
| | (0.320) | | (0.249) | | (0.231) | | (0.262) | | (0.246) | |
| Min, Max | 0.00, 3.00 | | 2.00, 3.00 | | 2.00, 3.00 | | 2.00, 3.00 | | 2.00, 3.00 | |
| Median | 2.55 | | 2.54 | | 2.50 | | 2.53 | | 2.55 | |
| Week 4 | | | | | | | | | | |
| N | 134 | 134 | 144 | 144 | 142 | 142 | 152 | 152 | 126 | 126 |
| Mean (SD) | 2.05 | −0.48 | 2.00 | −0.51 | 2.10 | −0.40 | 2.08 | −0.44 | 2.17 | −0.34 |
| | (0.567) | (0.547) | (0.597) | (0.563) | (0.522) | (0.469) | (0.580) | (0.514) | (0.446) | (0.386) |
| Min, Max | 0.00, 3.00 | −2.39, 2.00 | 0.00, 3.00 | −3.00, 0.58 | 0.00, 3.00 | −2.54, 0.52 | 0.00, 3.00 | −2.55, 0.38 | 1.00, 3.00 | −1.42, 0.80 |
| Median | 2.09 | −0.40 | 2.03 | −0.46 | 2.08 | −0.36 | 2.12 | −0.32 | 2.17 | −0.32 |
| Difference | | | | | | | | | | |
| from Placebo[1] | | | | | | | | | | |
| LS Mean | | −0.13 | | −0.17 | | −0.05 | | −0.10 | | |
| Standard Error | | 0.061 | | 0.060 | | 0.060 | | 0.059 | | |
| 95% CI | | (−0.25, | | (−0.28, | | (−0.17, | | (−0.21, | | |
| | | −0.01) | | −0.05) | | 0.07) | | 0.02) | | |
| P-value | | 0.031 | | 0.005 | | 0.401 | | 0.100 | | |
| Week 12 | | | | | | | | | | |
| N | 124 | 124 | 129 | 129 | 124 | 124 | 135 | 135 | 115 | 115 |
| Mean (SD) | 1.43 | −1.12 | 1.61 | −0.90 | 1.74 | −0.76 | 1.79 | −0.71 | 1.96 | −0.56 |
| | (0.977) | (0.963) | (0.817) | (0.783) | (0.756) | (0.744) | (0.814) | (0.806) | (0.618) | (0.603) |
| Min, Max | 0.00, 3.00 | −3.00, 0.47 | 0.00, 3.00 | −3.00, 0.44 | 0.00, 3.00 | −2.78, 0.71 | 0.00, 3.00 | −2.69, 1.00 | 0.00, 3.00 | −2.60, 0.83 |
| Median | 1.63 | −0.86 | 1.82 | −0.72 | 1.93 | −0.58 | 1.96 | −0.58 | 2.00 | −0.48 |
| Difference | | | | | | | | | | |
| from Placebo[1] | | | | | | | | | | |
| LS Mean | | −0.57 | | −0.39 | | −0.24 | | −0.16 | | |
| Standard Error | | 0.100 | | 0.099 | | 0.100 | | 0.098 | | |
| 95% CI | | (−0.77, | | (−0.59, | | (−0.43, | | (−0.36, | | |
| | | −0.38) | | −0.20) | | −0.04) | | 0.03) | | |
| P-value | | <.001 | | <.001 | | 0.018 | | 0.096 | | |

[1]Derived from the MMRM model with Treatment, Week (1-12), Treatment-by-Week interaction as factors, Baseline as covariate, and Subject as repeated measures unit. Difference is estimated from the simple contrast between the least squares means

TABLE 31
Change From Baseline to Weeks 4 and 12 in Severity Of Moderate To Severe Vasomotor Symptoms-LOCF
ANCOVA (MITT-VMS Population)

| | TRT1 (N = 141) | | TRT2 (N = 149) | | TRT3 (N = 147) | | TRT4 (N = 154) | | Placebo (N = 135) | |
|---|---|---|---|---|---|---|---|---|---|---|
| | | Change | | Change | | Change | | Change | | Change |
| | Actual | From | Actual | From | Actual | From | Actual | From | Actual | From |
| Study Week | Value | Baseline | Value | Baseline | Value | Baseline | Value | Baseline | Value | Baseline |
| Baseline | | | | | | | | | | |
| N | 141 | | 149 | | 147 | | 154 | | 135 | |
| Mean (SD) | 2.54 | | 2.51 | | 2.50 | | 2.51 | | 2.52 | |
| | (0.320) | | (0.249) | | (0.231) | | (0.262) | | (0.246) | |
| Min, Max | 0.00, 3.00 | | 2.00, 3.00 | | 2.00, 3.00 | | 2.00, 3.00 | | 2.00, 3.00 | |
| Median | 2.55 | | 2.54 | | 2.50 | | 2.53 | | 2.55 | |
| Week 4 | | | | | | | | | | |
| N | 141 | 141 | 149 | 149 | 147 | 147 | 154 | 154 | 135 | 135 |
| Mean (SD) | 2.07 | −0.44 | 2.18 | −0.34 | 2.07 | −0.47 | 2.01 | −0.51 | 2.11 | −0.39 |
| | (0.578) | (0.512) | (0.442) | (0.377) | (0.563) | (0.538) | (0.596) | (0.556) | (0.518) | (0.465) |
| Min, Max | 0.00, 3.00 | −2.39, 2.00 | 0.00, 3.00 | −3.00, 0.58 | 0.00, 3.00 | −2.54, 0.52 | 0.00, 3.00 | −2.55, 0.38 | 1.00, 3.00 | −1.42, 0.80 |
| Median | 2.11 | −0.39 | 2.03 | −0.45 | 2.09 | −0.35 | 2.10 | −0.32 | 2.16 | −0.32 |
| Difference | | | | | | | | | | |
| from Placebo[1] | | | | | | | | | | |
| LS Mean | | −0.12 | | −0.16 | | −0.05 | | −0.10 | | |
| Standard Error | | 0.059 | | 0.059 | | 0.059 | | 0.058 | | |
| 95% CI | | (−0.24, | | (−0.28, | | (−0.16, | | (−0.21, | | |
| | | −0.00) | | −0.05) | | 0.07) | | 0.02) | | |
| P-value | | 0.042 | | 0.006 | | 0.401 | | 0.093 | | |
| Week 12 | | | | | | | | | | |
| N | 141 | 141 | 149 | 149 | 147 | 147 | 154 | 154 | 135 | 135 |
| Mean (SD) | 1.47 | −1.07 | 1.60 | −0.91 | 1.75 | −0.75 | 1.81 | −0.70 | 1.99 | −0.53 |
| | (0.984) | (0.967) | (0.836) | (0.792) | (0.758) | (0.749) | (0.794) | (0.775) | (0.609) | (0.581) |
| Min, Max | 0.00, 3.00 | −3.00, 0.47 | 0.00, 3.00 | −3.00, 0.44 | 0.00, 3.00 | −2.78, 0.71 | 0.00, 3.00 | −2.69, 1.00 | 0.00, 3.00 | −2.60, 0.83 |
| Median | 1.67 | −0.81 | 1.81 | −0.74 | 1.92 | −0.56 | 1.95 | −0.57 | 2.07 | −0.44 |
| Difference | | | | | | | | | | |
| from Placebo[1] | | | | | | | | | | |
| LS Mean | | −0.54 | | −0.39 | | −0.23 | | −0.17 | | |
| Standard Error | | 0.094 | | 0.093 | | 0.093 | | 0.092 | | |
| 95% CI | | (−0.73, | | (−0.57, | | (−0.41, | | (−0.35, | | |
| | | −0.36) | | −0.20) | | −0.04) | | 0.01) | | |
| P-value | | <.001 | | <.001 | | 0.016 | | 0.061 | | |

[1]Derived from the ANCOVA model with Treatment as factor and Baseline as covariate.
Difference is estimated from the simple contrast between the least squares means.

TABLE 32
MENQOL Changes In Overall Scores From Baseline To Each Visit (Week 12, Month 6 And Month 12)
MITT-VMS Population

| MENQOL Domain | TRT1 (N = 141) | | TRT2 (N = 149) | | TRT3 (N = 147) | | TRT4 (N = 154) | | Placebo (N = 135) | |
|---|---|---|---|---|---|---|---|---|---|---|
| Study Week | | Change | | Change | | Change | | Change | | Change |
| Overall | Actual | From | Actual | From | Actual | From | Actual | From | Actual | From |
| MENQOL Score | Value | Baseline | Value | Baseline | Value | Baseline | Value | Baseline | Value | Baseline |
| Baseline | | | | | | | | | | |
| N | 140 | | 149 | | 147 | | 154 | | 135 | |
| Mean (SD) | 4.5 (1.17) | | 4.3 (1.25) | | 4.7 (1.44) | | 4.5 (1.27) | | 4.6 (1.34) | |
| Min, Max | 2, 7 | | 2, 8 | | 2, 8 | | 2, 8 | | 2, 8 | |
| Median | 4 | | 4 | | 4 | | 4 | | 5 | |
| Week 12 | | | | | | | | | | |
| N | 125 | 124 | 135 | 135 | 132 | 132 | 142 | 142 | 116 | 116 |
| Mean (SD) | 26 (1.13) | −1.9 (1.20) | 2.7 (1.26) | 2.8 (1.33) | 2.8 (1.33) | −1.9 (1.41) | 2.9 (1.36) | −1.7 (1.31) | 3.2 (1.29) | −1.4 (1.36) |
| Min, Max | 1, 7 | −5, 1 | 1, 7 | 1, 7 | −5, 2 | −6, 1 | 1, 7 | −6, 1 | 1, 7 | −5, 2 |
| Median | 2 | −2 | 2 | 2 | −1 | −2 | 3 | −1 | 3 | −1 |
| Difference from | | | | | | | | | | |
| Placebo[1] | | | | | | | | | | |
| LS Mean | | −0.56 | | −0.34 | | −0.47 | | −0.32 | | |
| Standard Error | | 0.143 | | 0.141 | | 0.141 | | 0.139 | | |
| 95% CI | | (−0.84, | | (−0.62, | | (0.74, | | (−0.59, | | |
| | | −0.28) | | −0.06) | | −0.19) | | −0.05) | | |
| P-value | | <.001 | | 0.016 | | <001 | | 0.021 | | |
| Month 6 | | | | | | | | | | |
| N | 117 | 116 | 130 | 130 | 118 | 118 | 126 | 126 | 104 | 104 |
| Mean (SD) | 2.4 (1.09) | −2.0 (1.22) | 2.4 (1.20) | −1.8 (1.22) | 2.6 (1.36) | −2.1 (1.50) | 2.7 (1.30) | −1.7 (1.24) | 3.0 (1.29) | −1.6 (1.31) |
| Min, Max | 1, 6 | −5, 1 | 1, 6 | −5, 1 | 1, 7 | −6, 1 | 1, 7 | −6, 1 | 1, 7 | −5, 1 |
| Median | 2 | −2 | 2 | −2 | 2 | −2 | 2 | −2 | 3 | −1 |
| Difference from | | | | | | | | | | |
| Placebo[1] | | | | | | | | | | |
| LS Mean | | −0.56 | | −0.43 | | −0.43 | | −0.22 | | |
| Standard Error | | 0.144 | | 0.142 | | 0.143 | | 0.141 | | |
| 95% CI | | (−0.84, | | (−0.71, | | (−0.71, | | (−0.49, | | |
| | | −0.27) | | −0.16) | | −0.15) | | 0.06) | | |
| P-value | | <.001 | | 0.002 | | 0.003 | | 0.124 | | |
| Month 12 | | | | | | | | | | |
| N | 126 | 125 | 134 | 134 | 134 | 134 | 130 | 130 | 124 | 124 |
| Mean (SD) | 2.7 (1.39) | −1.8 (1.41) | 2.3 (1.11) | 1.9 (1.35) | 2.9 (1.28) | −1.8 (1.51) | 2.9 (1.30) | −1.6 (1.33) | 3.2 (1.47) | −1.3 (1.50) |
| Min, Max | 1, 7 | −5, 2 | 1, 7 | −5, 3 | 1, 7 | −6, 1 | 1, 7 | −6, 2 | 1, 7 | −5.2 |
| Median | 2 | −2 | 2 | −2 | 3 | −2 | 3 | −2 | 3 | −1 |
| Difference from | | | | | | | | | | |
| Placebo[1] | | | | | | | | | | |
| LS Mean | | −0.48 | | −0.72 | | −0.38 | | −0.29 | | |
| Standard Error | | 0.152 | | 0.149 | | 0.149 | | 0.149 | | |
| 95% CI | | (−0.78, | | (−1.01, | | (−0.67, | | (−0.58, | | |
| | | −0.19) | | −0.42) | | −0.08) | | 0.01) | | |
| P-value | | 0.001 | | <.001 | | 0.012 | | 0.055 | | |

[1]Derived from the MMRM model with Treatment Week, Treatment-by-Week interaction as factors, Baseline as covariate, and Subject as repeated measures unit. Difference is estimated from the simple contrast between the least squares means.

TABLE 33
MENQOL Changes In Vasomotor Domain Score From Baseline To Each Visit (Week 12, Month 6 And Month 12)
(MITT-VMS Population)

| | TRT1 | | TRT2 | | TRT3 | | TRT4 | | Placebo | |
|---|---|---|---|---|---|---|---|---|---|---|
| | (N = 416) | | (N = 422) | | (N = 421) | | (N = 423) | | (N = 151) | |
| MENQOL | | Change | | Change | | Change | | Change | | Change |
| Domain | Actual | From | Actual | From | Actual | From | Actual | From | Actual | From |
| Study Week | Value | Baseline | Value | Baseline | Value | Baseline | Value | Baseline | Value | Baseline |
| Vasomotor | | | | | | | | | | |
| Domain | | | | | | | | | | |
| Baseline | | | | | | | | | | |
| N | 140 | | 149 | | 147 | | 154 | | 135 | |
| Mean (SD) | 7.1 | | 6.9 (1.06) | | 7.1 (1.00) | | 7.1 (0.96) | | 7.2 (1.00) | |
| Min, Max | 4, 8 | | 3, 8 | | 3, 8 | | 3, 8 | | 3, 8 | |
| Median | 7 | | 7 | | 7 | | 7 | | 7 | |
| Week 12 | | | | | | | | | | |
| N | 125 | 124 | 135 | 135 | 132 | 132 | 142 | 142 | 116 | 116 |
| Mean (SD) | 3.3 | −3.8 | 3.6 | −3.3 | 3.8 | −3.3 | 3.9 | −3.2 | 5.0 | −2.2 |
| | (1.93) | (1.98) | (1.91) | (2.04) | (1.89) | (1.97) | (2.07) | (2.13) | (1.79) | (1.83) |
| Min, Max | 1, 8 | −7, 2 | 1, 8 | −7, 3 | 1, 8 | −7, 2 | 1, 8 | −7, 1 | 1, 8 | −7, 3 |
| Median | 3 | −4 | 3 | −4 | 4 | −4 | 4 | −3 | 5 | −2 |
| Difference from | | | | | | | | | | |
| Placebo[1] | | | | | | | | | | |
| LS Mean | | | −1.68 | | −1.37 | | −1.19 | | −1.09 | |
| Standard Error | | | 0.243 | | 0.239 | | 0.239 | | 0.236 | |
| 95% CI | | | (−2.16, | | (−1.84, | | (−1.66, | | (−1.55, | |
| | | | −1.20) | | −0.90) | | −0.72) | | −0.62) | |
| P-value | | | <.001 | | <.001 | | <.001 | | <.001 | |
| Month 6 | | | | | | | | | | |
| N | 117 | 116 | 130 | 130 | 118 | 118 | 126 | 126 | 104 | 104 |
| Mean (SD) | 2.8 | −4.3 | 2.9 | −4.1 | 3.1 | −4.0 | 3.5 | −3.5 | 4.2 | −3.0 |
| | | (1.94) | (1.75) | (1.99) | (1.99) | (2.12) | (1.93) | (1.95) | (2.12) | (2.26) |
| Min, Max | 1, 7 | −7, 1 | 1, 8 | −7, 1 | 1, 8 | −7, 1 | 1, 8 | −7, 1 | 1, 8 | −7, 2 |
| Median | 2 | −5 | 2 | −5 | 3 | −4 | 3 | −4 | 4 | −3 |
| Difference from | | | | | | | | | | |
| Placebo[1] | | | | | | | | | | |
| LS Mean | | | −1.48 | | −1.41 | | −1.16 | | −0.80 | |
| Standard Error | | | 0.251 | | 0.246 | | 0.249 | | 0.245 | |
| 95% CI | | | (−1.97, | | (−1.89, | | (−1.64, | | (−1.28, | |
| | | | −0.98) | | −0.92) | | −0.67) | | −0.32) | |
| P-value | | | <.001 | | <.001 | | <.001 | | 0.001 | |
| Month 12 | | | | | | | | | | |
| N | 126 | 125 | 134 | 134 | 134 | 134 | 130 | 130 | 123 | 123 |
| Mean (SD) | 3.2 | −3.9 | 2.9 | −4.0 | 3.5 | −3.6 | 3.8 | −3.2 | 4.7 | −2.4 |
| | (2.13) | (2.18) | (1.67) | (1.90) | (2.14) | (2.26) | (1.99) | (1.87) | (2.21) | (2.31) |
| Min, Max | 1, 8 | −7, 2 | 1, 8 | −7, 2 | 1, 8 | −7, 2 | 1, 8 | −7, 1 | 1, 8 | −7, 3 |
| Median | 3 | −4 | 3 | −4 | 3 | −3 | 4 | −3 | 5 | −2 |
| Difference from | | | | | | | | | | |
| Placebo[1] | | | | | | | | | | |
| LS Mean | | | −1.53 | | −1.69 | | −1.19 | | −0.87 | |
| Standard Error | | | 0.252 | | 0.248 | | 0.248 | | 0.248 | |
| 95% CI | | | (−2.02, | | (−2.18, | | (−1.68, | | (−1.36, | |
| | | | −1.03) | | −1.20) | | −0.70) | | −0.38) | |
| P-value | | | <.001 | | <.001 | | <.001 | | <.001 | |

[1]Derived from the MMRM model with Treatment, Week, Treatment-by-Week interaction as factors, Baseline as covariate, and Subject as repeated measures unit. Difference is estimated from the simple contrast between the least squares means.

TABLE 34
MENQOL Changes In Overall Score From Baseline To Each Visit (Week 12, Month 6 And Month 12)
(MITT Population)

| | TRT1 | | TRT2 | | TRT3 | | TRT4 | | Placebo | |
|---|---|---|---|---|---|---|---|---|---|---|
| MENQOL Domain | (N = 416) | | (N = 422) | | (N = 421) | | (N = 423) | | (N = 151) | |
| Study Week | | Change | | Change | | Change | | Change | | Change |
| Overall | Actual | From | Actual | From | Actual | From | Actual | From | Actual | From |
| MENQOL Score | Value | Baseline | Value | Baseline | Value | Baseline | Value | Baseline | Value | Baseline |
| Baseline | | | | | | | | | | |
| N | 414 | | 422 | | 421 | | 422 | | 151 | |
| Mean (SD) | 4.3 | | 4.3 (1.30) | | 4.4 (1.40) | | 4.3 (1.33) | | 4.7 (1.37) | |
| Min, Max | 2, 8 | | 2, 8 | | 2, 8 | | 2, 8 | | 2, 8 | |
| Median | 4 | | 4 | | 4 | | 4 | | 5 | |
| Week 12 | | | | | | | | | | |
| N | 355 | 354 | 364 | 364 | 375 | 375 | 377 | 376 | 117 | 117 |
| Mean (SD) | 2.5 (1.13) | −1.8 (1.31) | 2.6 (1.14) | −1.6 (1.38) | 2.6 (1.22) | −1.7 (1.35) | 2.7 (1.22) | −1.6 (1.35) | 3.2 (1.30) | −1.4 (1.36) |
| Min, Max | 1, 7 | −6, 1 | 1, 7 | −6, 2 | 1, 8 | −6, 1 | 1, 7 | −6, 2 | 1, 7 | −5, 2 |
| Median | 2 | −2 | 2 | −1 | 2 | −2 | 3 | −1 | 3 | −1 |
| Change from | | | | | | | | | | |
| Baseline[1] | | | | | | | | | | |
| 95% CI | | (−1.93, | | (−1.77, | | (−1.87, | | (−1.76, | | (−1.60, |
| | | −1.65) | | −1.48) | | −1.60) | | −1.48) | | −1.10) |
| P-value | | <0.001 | | <0.001 | | <0.001 | | <0.001 | | <0.001 |
| Month 6 | | | | | | | | | | |
| N | 317 | 316 | 333 | 333 | 343 | 343 | 325 | 324 | 104 | 104 |
| Mean (SD) | 2.3 | −2.0 | 2.4 (1.10) | −1.8 (1.38) | 2.5 (1.25) | −1.8 (1.39) | 2.6 (1.24) | −1.7 (1.29) | 3.0 (1.29) | −1.6 (1.31) |
| | (1.01) | (1.32) | | | | | | | | |
| Min, Max | 1, 6 | −6, 1 | 1, 6 | −6, 3 | 1, 7 | −6, 2 | 1, 7 | −6, 2 | 1, 7 | −5, 1 |
| Median | 2 | −2 | 2 | −2 | 2 | −2 | 2 | −2 | 3 | −1 |
| Change from | | | | | | | | | | |
| Baseline[1] | | | | | | | | | | |
| 95% CI | | (−2.13, | | (−1.96, | | (−1.92, | | (−1.79, | | (−1.85, |
| | | −1.84) | | −1.67) | | −1.62) | | −1.51) | | −1.34) |
| P-value | | <0.001 | | <0.001 | | <0.001 | | <0.001 | | <0.001 |
| Month 12 | | | | | | | | | | |
| N | 367 | 365 | 365 | 365 | 381 | 381 | 361 | 360 | 125 | 125 |
| Mean (SD) | 2.5 (1.25) | −1.8 (1.44) | 2.5 (1.15) | −1.7 (1.45) | 2.7 (1.25) | −1.6 (1.40) | 2.9 (1.38) | −1.5 (1.41) | 3.2 (1.47) | −1.3 (1.50) |
| Min, Max | 1, 7 | −6, 3 | 1, 7 | −6, 3 | 1, 8 | −6, 4 | 1, 8 | −6, 2 | 1, 7 | −5, 2 |
| Median | 2 | −2 | 2 | −2 | 2 | −2 | 3 | −1 | 3 | −1 |
| Change from | | | | | | | | | | |
| Baseline[1] | | | | | | | | | | |
| 95% CI | | (−1.95, | | (−1.89, | | (−1.78, | | (−1.65, | | (−1.59, |
| | | −1.66) | | −1.59) | | −1.50) | | −1.36) | | −1.06) |
| P-value | | <0.001 | | <0.001 | | <0.001 | | <0.001 | | <0.001 |

[1]Paired t-test for the mean change from baseline.

TABLE 35
MENQOL Changes In Vasomotor Domain Score From Baseline To Each Visit (Week 12, Month 6 And Month 12)
(MITT Population)

| MENQOL | TRT1 | | TRT2 | | TRT3 | | TRT4 | | Placebo | |
|---|---|---|---|---|---|---|---|---|---|---|
| Domain | (N = 416) | | (N = 422) | | (N = 421) | | (N = 423) | | (N = 151) | |
| Study Week | | Change | | Change | | Change | | Change | | Change |
| Vasomotor | Actual | From | Actual | From | Actual | From | Actual | From | Actual | From |
| Domain | Value | Baseline | Value | Baseline | Value | Baseline | Value | Baseline | Value | Baseline |
| Baseline | | | | | | | | | | |
| N | 414 | | 422 | | 421 | | 422 | | 151 | |
| Mean (SD) | 6.3 (1.45) | | 6.4 (1.30) | | 6.4 (1.40) | | 6.4 (1.33) | | 7.1 (0.99) | |
| Min, Max | 1, 8 | | 2, 8 | | 2, 8 | | 2, 8 | | 3, 8 | |
| Median | 7 | | 7 | | 7 | | 7 | | 7 | |
| Week 12 | | | | | | | | | | |
| N | 355 | 354 | 364 | 364 | 375 | 375 | 377 | 376 | 117 | 117 |
| Mean (SD) | 2.8 (1.75 | −3.5 (2.02) | 3.3 (1.79) | −3.1 (2.00) | 3.3 (1.85) | −3.1 (2.01) | 3.5 (1.86) | −2.9 (1.94) | 5.0 (1.80) | −2.2 (1.84) |
| Min, Max | 1, 8 | −7, 2 | 1, 8 | −7, 5 | 1, 8 | −7, 2 | 1, 8 | −7, 3 | 1, 8 | −7, 3 |
| Median | 2 | −4 | 3 | −3 | 3 | −3 | 3 | −3 | 5 | −2 |
| Difference | | | | | | | | | | |
| from Placebo[1] | | | | | | | | | | |
| LS Mean | | −1.96 | | −1.50 | | −1.49 | | −1.31 | | |
| Standard Error | | 0.188 | | 0.187 | | 0.187 | | 0.187 | | |
| 95% CI | | (−2.33, | | (−1.87, | | (−1.86, | | (−1.67, | | |
| | | −1.59) | | −1.13) | | −1.13) | | −0.94) | | |
| P-value | | <.001 | | <.001 | | <.001 | | <.001 | | |
| Month 6 | | | | | | | | | | |
| N | 317 | 316 | 333 | 333 | 342 | 342 | 325 | 324 | 104 | 104 |
| Mean (SD) | 2.3 (1.53) | −4.0 (1.95) | 2.8 (1.69) | −3.7 (2.02) | 2.9 (1.85) | −3.5 (2.15) | 3.1 (1.80) | −3.3 (1.94) | 4.2 (2.12) | −3.0 (2.26) |
| Min, Max | 1, 7 | −7, 3 | 1, 8 | −7, 3 | 1, 8 | −7, 2 | 1, 8 | −7, 2 | 1, 8 | −7, 2 |
| Median | 2 | −4 | 2 | −4 | 2 | −4 | 3 | −4 | 4 | −3 |
| Difference | | | | | | | | | | |
| from Placebo[1] | | | | | | | | | | |
| LS Mean | | −1.70 | | −1.33 | | −1.20 | | −0.94 | | |
| Standard Error | | 0.193 | | 0.192 | | 0.192 | | 0.192 | | |
| 95% CI | | (−2.08, | | (−1.70, | | (−1.58, | | (−1.31, | | |
| | | −1.32) | | −0.95) | | −0.82) | | −0.56) | | |
| P-value | | <.001 | | <.001 | | <.001 | | <.001 | | |
| Month 12 | | | | | | | | | | |
| N | 367 | 365 | 365 | 365 | 381 | 381 | 360 | 359 | 124 | 124 |
| Mean (SD) | 2.7 (1.86) | −3.6 (2.15) | 2.9 (1.74) | −3.5 (2.00) | 3.1 (1.96) | −3.2 (2.14) | 3.5 (1.98) | −2.9 (1.98) | 4.7 (2.22) | −2.4 (2.31) |
| Min, Max | 1, 8 | −7, 3 | 1, 8 | −7, 2 | 1, 8 | −7, 3 | 1, 8 | −7, 3 | 1, 8 | −7, 3 |
| Median | 2 | −4 | 3 | −4 | 3 | −3 | 3 | −3 | 5 | −2 |
| Difference | | | | | | | | | | |
| from Placebo[1] | | | | | | | | | | |
| LS Mean | | −1.80 | | −1.62 | | −1.42 | | −1.10 | | |
| Standard Error | | 0.194 | | 0.193 | | 0.193 | | 0.193 | | |
| 95% CI | | (−2.18, | | (−2.00, | | (−1.80, | | (−1.48, | | |
| | | −1.42) | | −1.24) | | −1.04) | | −0.72) | | |
| P-value | | <.001 | | <.001 | | <.001 | | <.001 | | |

[1]Derived from the MMRM model with Treatment, Week, Treatment-by-Week interaction as factors, Baseline as covariate, and Subject as repeated measures unit.
Difference is estimated from the simple contrast between the least squares means.

TABLE 36 Summary of Percent Treatment Responders at Weeks 4, 8, And 12 Based On Subject Satisfaction with Treatment
(Clinical Global Impression [CGI]) Compared to Changes in Frequency of Moderate to Severe Vasomotor Symptoms from
Baseline (MITT-VMS Population)

| | TRT1 | | TRT2 | | TRT3 | | TRT4 | | Placebo | |
|---|---|---|---|---|---|---|---|---|---|---|
| Clinical | (N = 141) | | (N = 149) | | (N = 147) | | (N = 154) | | (N = 135) | |
| Global | | Mean | | Mean | | Mean | | Mean | | Mean |
| Impression | N (%) [1] | Chg [2] | N (%) [1] | Chg [2] | N (%) [1] | Chg [2] | N (%) [1] | Chg [2] | N (%) [1] | Chg [2] |
| Week 4 | | | | | | | | | | |
| Very Much | 42/136 (30.9) | −56.5 | 29/141 (20.6) | −53.5 | 23/144 (16.0) | −47.8 | 28/148 (18.9) | −63.2 | 8/125 (6.4) | −53.5 |
| Improved | | | | | | | | | | |
| Much | 44/136 (32.4) | −46.9 | 42/141 (29.8) | −44.4 | 49/144 (34.0) | −44.5 | 47/148 (31.8) | −50.5 | 33/125 (26.4) | −46.8 |
| Improved | | | | | | | | | | |
| Minimally | 37/136 (27.2) | −24.1 | 49/141 (34.8) | −25.9 | 49/144 (34.0) | −28.8 | 51/148 (34.5) | −28.9 | 49/125 (39.2) | −27.2 |
| Improved | | | | | | | | | | |
| No Change | 11/136 (8.1) | −14.9 | 21/141 (14.9) | −6.6 | 15/144 (10.4) | −6.3 | 18/148 (12.2) | −3.4 | 33/125 (26.4) | −5.6 |
| Minimally | 2/136 (1.5) | 24.5 | 0 | | 4/144 (2.8) | 1.2 | 3/148 (2.0) | −7.7 | 2/125 (1.6) | 1.0 |
| Worse | | | | | | | | | | |
| Much Worse | 0 | | 0 | | 2/144 (1.4) | −3.5 | 0 | | 0 | |
| Very Much | 0 | | 0 | | 2/144 (1.4) | 14.5 | 1/148 (0.7) | −31.0 | 0 | |
| Worse | | | | | | | | | | |
| ***P- | <0.001 | | 0.004 | | 0.005 | | 0.003 | | | |
| Value[3] *** | | | | | | | | | | |
| Week 8 | | | | | | | | | | |
| Very Much | 64/130 (49.2) | −65.5 | 55/139 (39.6) | −61.6 | 50/134 (37.3) | −62.3 | 38/141 (27.0) | −64.1 | 26/117 (22.2) | −62.1 |
| Improved | | | | | | | | | | |
| Much | 37/130 (28.5) | −49.9 | 48/139 (34.5) | −46.7 | 48/134 (35.8) | −50.2 | 55/141 (39.0) | −53.5 | 36/117 (30.8) | −44.2 |
| Improved | | | | | | | | | | |
| Minimally | 23/130 (17.7) | −28.7 | 24/139 (17.3) | −27.3 | 23/134 (17.2) | −27.2 | 35/141 (24.8) | −36.8 | 25/117 (21.4) | −28.5 |
| Improved | | | | | | | | | | |
| No Change | 6/130 (4.6) | −0.2 | 8/139 (5.8) | −12.0 | 8/134 (6.0) | 0.0 | 11/141 (7.8) | −11.9 | 24/117 (20.5) | −4.5 |
| Minimally | 0 | | 4/139 (2.9) | −21.5 | 3/134 (2.2) | −1.7 | 1/141 (0.7) | 0.0 | 5/117 (4.3) | −21.9 |
| Worse | | | | | | | | | | |
| Much Worse | 0 | | 0 | | 1/134 (0.7) | −29.0 | 0 | 0 | | |
| Very Much | 0 | | 0 | | 1/134 (0.7) | 13.0 | 1/141 (0.7) | 35.0 | 1/117 (0.9) | 5.0 |
| Worse | | | | | | | | | | |
| ***P- | <0.001 | | <0.001 | | 0.001 | | 0.041 | | | |
| Value[3] *** | | | | | | | | | | |
| Week 12 | | | | | | | | | | |
| Very Much | 54/123 (43.9) | −66.3 | 54/133 (40.6) | −66.3 | 47/131 (35.9) | −68.5 | 52/139 (37.4) | −67.0 | 29/116 (25.0) | −68.4 |
| Improved | | | | | | | | | | |
| Much | 47/123 (38.2) | −51.8 | 43/133 (32.3) | −51.7 | 55/131 (42.0) | −48.6 | 49/139 (35.3) | −52.4 | 33/116 (28.4) | −43.1 |
| Improved | | | | | | | | | | |
| Minimally | 17/123 (13.8) | −35.5 | 29/133 (21.8) | −34.7 | 22/131 (16.8) | −28.8 | 24/139 (17.3) | −39.1 | 26/116 (22.4) | −35.7 |
| Improved | | | | | | | | | | |
| No Change | 4/123 (3.3) | −26.6 | 6/133 (4.5) | −40.8 | 4/131 (3.1) | −7.3 | 10/139 (7.2) | −11.6 | 22/116 (19.0) | −9.9 |
| Minimally | 1/123 (0.8) | −47.0 | 1/133 (0.8) | −27.0 | 2/131 (1.5) | −9.5 | 2/139 (1.4) | −25.0 | 5/116 (4.3) | −9.8 |
| Worse | | | | | | | | | | |
| Much Worse | 0 | | 0 | | 1/131 (0.8) | −12.0 | 2/139 (1.4) | 36.0 | 1/116 (0.9) | −13.0 |
| ***P-Value | <0.001 | | 0.002 | | <0.001 | | 0.002 | | | |

TABLE 37
Mean Change in Frequency and Severity of Hot Flashes

| Estradiol/Progesterone | | | | |
|---|---|---|---|---|
| 1 mg/ | 0.5 mg/ | 0.5 mg/ | 0.25 mg/ | |
| 100 mg | 100 mg | 50 mg | 50 mg | Placebo |

Replenish Trial Co-Primary Efficacy Endpoints: Mean Change in Frequency and Severity
of Hot Flashes Per Week Versus Placebo at Weeks 4 and 12, VMS-MITT Population
Baseline

| | | | | | |
|---|---|---|---|---|---|
| | n = 141 | n = 149 | n = 147 | n = 154 | n = 135 |
| Frequency (Mean) | 74.4 | 72.1 | 75.9 | 77.0 | 72.4 |
| Severity (Mean) | 2.54 | 2.51 | 2.50 | 2.51 | 2.52 |

Week 4

| | | | | | |
|---|---|---|---|---|---|
| Frequency (Mean) | 31.5 | 37.2 | 41.5 | 38.4 | 45.9 |
| Difference from placebo | −12.81 | −8.07 | −4.81 | −10.40 | — |
| (LS Mean)* | | | | | |
| P-value versus placebo | <0.001 | 0.013 | 0.141 | 0.001 | — |
| Severity (Mean) | 2.05 | 2.00 | 2.10 | 2.08 | 2.17 |
| P-value versus placebo | 0.031 | 0.005 | 0.401 | 0.1 | — |

Week 12

| | | | | | |
|---|---|---|---|---|---|
| Frequency (Mean) | 17.1 | 19.1 | 25.2 | 24.1 | 32.0 |
| Difference from placebo | −16.58 | −15.07 | −10.79 | −11.71 | — |
| (LS Mean) | | | | | |
| P-value versus placebo | <0.001 | <0.001 | 0.002 | <0.001 | — |
| Severity (Mean) | 1.43 | 1.61 | 1.74 | 1.79 | 1.96 |
| P-value versus placebo | <0.001 | <0.001 | 0.018 | 0.096 | — |

Replenish Trial Primary Safety Endpoint: Incidence of Consensus Endometrial
Hyperplasia or Malignancy up to 12 months, Endometrial Safety Population^{∓}

| | | | | | |
|---|---|---|---|---|---|
| Endometrial Hyperplasia | 0% (0/280) | 0/303 (0%) | 0/306 (0%) | 0/274 (0%) | 0/92 (0%) |

MITT = Modified intent to treat
^{∓}Per FDA, consensus hyperplasia refers to the concurrence of two of the three pathologists be accepted as the final diagnosis ¹
*Least Squares Means derived from the MMRM model

Based on the results, it is clear that multiple doses of doses of TX-001HR studied in the Replenish Trial provided positive results, demonstrating that that this drug product candidate is capable of addressing the significant demand for bio-identical hormone therapy. Over the past 14 years, women in the U.S. have moved from synthetic FDA-approved hormone therapy to unapproved bio-identical hormones mixed together or compounded at independent or community based pharmacies. The goal of the Replenish Trial was to provide a proven safe and effective bio-identical combination hormone therapy to meet the needs of women, physicians and pharmacies, that have largely been ignored by the pharmaceutical industry, and it has been found that TX-001HR at multiple different doses does just that. TX-001HR, quite importantly, provides the hormonal solution that women demand with proven safety and efficacy.

TX-001HR is the first bio-identical hormone therapy of estradiol in combination with natural progesterone to be evaluated in a large, well-controlled, randomized clinical trial. The Replenish Trial demonstrated for the first-time safety and robust efficacy for the treatment of hot flashes of multiple doses of estradiol in combination with natural progesterone with a consistency of effect noted on the primary and secondary efficacy endpoints. Importantly, TX-001HR establishes the FDA required endometrial safety for multiple doses of estradiol in combination with natural progesterone in continuous combined regimen.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices or methods. This disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad general meaning of the terms in which the appended claims are expressed. To the extent that these various modifications do not depart from the spirit and scope of the appended claims, they are intended to be encompassed therein.

1. A pharmaceutical composition for co-administering estradiol and progesterone to a human subject having vasomotor symptoms associated with estrogen deficiency comprising: 1 mg of estradiol and 100 mg of progesterone;
wherein administration of the composition to the subject produces, in a serum sample from the subject, one or more pharmacokinetic parameters selected from:
(i) a steady state estradiol concentration that is from 42.29 pg/mL to 45.58 pg/mL;
(ii) a steady state estrone concentration that is from 213.79 pg/mL to 241.57 pg/mL; and
(iii) a steady state progesterone concentration that is from 386.53 pg/mL to 547.83 pg/mL.

2. The pharmaceutical composition of claim 1, wherein administration of the composition to the subject produces a steady state estradiol concentration that is from 42.29 pg/mL to 45.58 pg/mL.

3. (canceled)

4. The pharmaceutical composition of claim 1, wherein administration of the composition to the subject produces both a steady state estradiol concentration that is from 42.29 pg/mL to 45.58 pg/mL and a steady state progesterone concentration that is from 386.53 pg/mL to 547.83 pg/mL.

5. (canceled)

6. A pharmaceutical composition for co-administering estradiol and progesterone to a human subject having vasomotor symptoms associated with estrogen deficiency comprising: 0.5 mg of estradiol and 100 mg of progesterone;
wherein administration of the composition to the subject produces, in a serum sample from the subject, one or more pharmacokinetic parameters selected from:
(i) a steady state estradiol concentration that is from 23.03 pg/mL to 27.37 pg/mL;
(ii) a steady state estrone concentration that is from 113.59 pg/mL to 132.08 pg/mL; and
(iii) a steady state progesterone concentration that is from 386.53 pg/mL to 547.83 pg/mL.

7. The pharmaceutical composition of claim 6, wherein administration of the composition to the subject produces a steady state estradiol concentration that is from 23.03 pg/mL to 27.37 pg/mL.

8. (canceled)

9. The pharmaceutical composition of claim 6, wherein administration of the composition to the subject produces both a steady state estradiol concentration that is from 23.03 pg/mL to 27.37 pg/mL and a steady state progesterone concentration that is from 386.53 pg/mL to 547.83 pg/mL.

10. (canceled)

11. A pharmaceutical composition for co-administering estradiol and progesterone to a human subject having vasomotor symptoms associated with estrogen deficiency comprising: 0.5 mg of estradiol and 50 mg of progesterone;
wherein administration of the composition to the subject produces, in a serum sample from the subject, one or more pharmacokinetic parameters selected from:
(i) a steady state estradiol concentration that is from 23.03 pg/mL to 27.37 pg/mL;
(ii) a steady state estrone concentration that is from 113.59 pg/mL to 132.08 pg/mL; and
(iii) a steady state progesterone concentration that is from 181.41 pg/mL to 247.17 pg/mL.

12. The pharmaceutical composition of claim 11, wherein administration of the composition to the subject produces a steady state estradiol concentration that is from 23.03 pg/mL to 27.37 pg/mL.

13. (canceled)

14. The pharmaceutical composition of claim 11, wherein administration of the composition to the subject produces both a steady state estradiol concentration that is from 23.03 pg/mL to 27.37 pg/mL and a steady state progesterone concentration that is from 181.41 pg/mL to 247.17 pg/mL.

15. (canceled)

16. A pharmaceutical composition for co-administering estradiol and progesterone to a human subject having vasomotor symptoms associated with estrogen deficiency comprising: 0.25 mg of estradiol and 50 mg of progesterone;
wherein administration of the composition to the subject produces, in a serum sample from the subject, one or more pharmacokinetic parameters selected from:
(i) a steady state estradiol concentration that is from 15.06 pg/mL to 18.50 pg/mL;
(ii) a steady state estrone concentration that is from 69.02 pg/mL to 73.43 pg/mL; and
(iii) a steady state progesterone concentration that is from 181.41 pg/mL to 247.17 pg/mL.

17. The pharmaceutical composition of claim 16, wherein administration of the composition to the subject produces a steady state estradiol concentration that is from 15.06 pg/mL to 18.50 pg/mL.

18. (canceled)

19. The pharmaceutical composition of claim 16, wherein administration of the composition to the subject produces both a steady state estradiol concentration that is from 15.06 pg/mL to 18.50 pg/mL and a steady state progesterone concentration that is from 181.41 pg/mL to 247.17 pg/mL.

20. (canceled)

21. The pharmaceutical composition of claim 1, wherein composition further comprises a solubilizing agent.

22. The pharmaceutical composition of claim 21, wherein the solubilizing agent comprises a medium chain (C6-C12) oil.

23. (canceled)

24. The pharmaceutical composition of claim 1, wherein the estradiol is solubilized estradiol.

25. (canceled)

26. The pharmaceutical composition of claim 1, wherein the progesterone is suspended progesterone.

27-28. (canceled)

29. The pharmaceutical composition of claim 1, wherein administration of the composition to the subject further produces in the subject a reduction in the frequency and/or severity of one or more symptoms of menopause.

30-32. (canceled)

33. A method of treating a subject having vasomotor symptoms associated with estrogen deficiency, the method comprising administering to the subject an effective amount of the pharmaceutical composition of claim 1.

34. The method of claim 33, wherein the subject is female.

35. (canceled)

36. The method of claim 33, wherein prior to treatment, the subject has a serum estradiol of ≤10 pg/mL.

37. (canceled)
